(19) United States
(12) Patent Application Publication
(10) Pub. No.: US 2013/0017199 A1
(43) Pub. Date: Jan. 17, 2013

(54) SIMULTANEOUS INHIBITION OF PD-L1/PD-L2

(71) Applicant: Solomon Langermann, Baltimore, MD (US)

(72) Inventor: Solomon Langermann, Baltimore, MD (US)

(73) Assignee: AMPLIMMUNE ,Inc. a corporation

(21) Appl. No.: 13/511,879

(22) PCT Filed: Nov. 24, 2010
(86) PCT No.: PCT/US2010/057940

§ 371 Date: May 24, 2012

Related U.S. Application Data
(60) Provisional application No. 61/263,983, filed on Nov. 24, 2009.

(51) Int. Cl. A61K 39/395; A61P 35/00; A61P 33/00; A61P 31/04; A61P 31/10; A61P 37/02; A61P 31/12

(52) U.S. Cl. 424/134.1

(57) ABSTRACT

Methods and compositions for treating an infection or disease that results from (1) failure to elicit rapid T cell mediated responses, (2) induction of T cell exhaustion, T cell anergy or both, or (3) failure to activate monocytes, macrophages, dendritic cells and/or other APCs, for example, as required to kill intracellular pathogens. The method and compositions solve the problem of undesired T cell inhibition by simultaneously inhibiting the PD-1 ligands, PD-L1 and PD-L2. The immune response can be modulated by providing antagonists which bind with different affinity, by varying the dosage of agent which is administered, by intermittent dosing over a regime, and combinations thereof, that provides for dissociation of agent from the molecule to which it is bound prior to being administered again. In some cases it may be particularly desirable to stimulate the immune system, then remove the stimulation.

### FIELD OF THE INVENTION

The invention generally relates to immunomodulatory compositions and methods for treating diseases such as cancer or infections, in particular to diseases inducing T cell exhaustion, T cell anergy, or both, or diseases where intracellular pathogens e.g., Leishmania, evade immune response by upregulating PD-1 ligands on APCs (e.g. monocytes, dendritic cells, macrophages) or epithelial cells.

### BACKGROUND OF THE INVENTION

Cancer has an enormous physiological and economic impact. For example a total of 1,437,180 new cancer cases and 565,650 deaths from cancer are projected to occur in the United States in 2008 (Jemal, A., Cancer J. Clin., 58:71-96 (2008)). The National Institutes of Health estimate overall costs of cancer in 2007 at $219.2 billion: $89.0 billion for direct medical costs (total of all health expenditures); $18.2 billion for indirect morbidity costs (cost of lost productivity due to illness); and $112.0 billion for indirect mortality costs (cost of lost productivity due to premature death). Although there are several methods for treating cancer, each method has its own degree of effectiveness as well as side-effects. Typical methods for treating cancer include surgery, chemotherapy, radiation, and immunotherapy.

Stimulating the patients own immune response to target tumor cells is an attractive option for cancer therapy and many studies have demonstrated effectiveness of immunotherapy using tumor antigens to induce the immune response. However, induction of an immune response and the effective eradication of cancer often do not correlate in cancer immunotherapy trials (Cormier, et al., Cancer J. Sci. Am., 3(1):37-44 (1997); Nestle, et al., Nat. Med., 4(3):328-332 (1998); Rosenberg, Nature, 411(6835):380-384 (2001)). Thus, despite primary anti-tumor immune responses in many cases, functional, effector anti-tumor T cell responses are often weak at best.

Antigen-specific activation and proliferation of lymphocytes are regulated by both positive and negative signals from costimulatory molecules. The most extensively characterized T cell costimulatory pathway is B7-CD28, in which B7-1 (CD80) and B7-2 (CD86) each can engage the stimulatory CD28 receptor and the inhibitory CTLA-4 (CD152) receptor. In conjunction with signaling through the T cell receptor, CD28 ligation increases antigen-specific proliferation of T cells, enhances production of cytokines, stimulates differentiation and effector function, and promotes survival of T cells (Lenshow, et al., Annu. Rev. Immunol., 14:233-258 (1996); Chambers and Allison, Curr. Opin. Immunol., 9:396-404 (1997); and Rathmell and Thompson, Annu. Rev. Immunol., 17:781-828 (1999)). In contrast, signaling through CTLA-4 is thought to deliver a negative signal that inhibits T cell proliferation, IL-2 production, and cell cycle progression (Krummel and Allison, J. Exp. Med., 183:2533-2540 (1996); and Walunas, et al., J. Exp. Med., 183:2541-2550 (1996)). Other members of the B7 family include B7-H1 (Dong, et al., Nature Med., 5:1365-1369 (1999); and Freeman, et al., J. Exp. Med., 192:1-9 (2000)), B7-DC (Tseng, et al., J. Exp. Med., 193:839-846 (2001); and Latchman, et al., Nature Immunol., 2:261-268 (2001)), B7-H2 (Wang, et al., Blood, 96:2808-2813 (2000); Swallow, et al., Immunity, 11:423-432 (1999); and Yoshinaga, et al., Nature, 402:827-832 (1999)), B7-H3 (Chapoval, et al., Nature Immunol., 2:269-274 (2001)) and B7-H4 (Choi, et al., J. Immunol., 171:4650-4654 (2003); Sica, et al., Immunity, 18:849-861 (2003); Prasad, et al., Immunity, 18:863-873 (2003); and Zang, et al., Proc. Natl. Acad. Sci. U.S.A., 100:10388-10392 (2003)).

PD-L1 and PD-L2 are ligands for PD-1 (programmed cell death-1), B7-H2 is a ligand for ICOS, and B7-H3, B7-H4 and B7-H5 remain orphan ligands at this time (Dong, et al., Immunol. Res., 28:39-48 (2003)).

The primary result of PD-1 ligation by its ligands is to inhibit signaling downstream of the T cell Receptor (TCR). Therefore, signal transduction via PD-1 usually provides a suppressive or inhibitory signal to the T cell that results in decreased T cell proliferation or other reduction in T cell activation. PD-1 signaling is thought to require binding to a PD-1 ligand in close proximity to a peptide antigen presented by major histocompatibility complex (MHC), which is bound to the TCR (Freeman, Proc. Natl. Acad. Sci. U.S.A, 105:10275-10276 (2008)). PD-L1 is the predominant PD-1 ligand causing inhibitory signal transduction in T cells.

T cells can also be inhibited by T regulatory cells (Tregs)(Schwartz, R., Nature Immunology, 6:327-330 (2005)). Tregs have been shown to suppress tumor-specific T cell immunity, and may contribute to the progression of human tumors (Liyanage, U. K., et al., J Immunol, 169:2756-2761 (2002). In mice, depletion of Treg cells leads to more efficient tumor rejection (Viehl, C. T., et al., Ann Surg Oncol, 13:1252-1258 (2006)).

Thus, it is an object of the invention to provide an immunomodulatory composition that blocks both PD-L1 and PD-L2 mediated signal transduction. and enhance immune responses.

It is another object to provide compositions that induce robust effector responses and reduced Treg responses against tumors and chronic infections.

It is another object of the invention to provide compositions and methods for increasing the number of Th17 cells and/or the level of IL-17 production at the site of a tumor or a pathogen infected area.

It is another object of the invention to provide compositions and methods for reducing the number of PD-1 positive cells at the site of a tumor or a pathogen infected area.

It is another object to provide compositions and methods for treating infections that induce T cell exhaustion, T cell anergy, or both.

It is yet another object of the invention to provide compositions and methods for treating intracellular infections of antigen presenting cells, including monocytes, dendritic cells, and macrophages.

It is another object of the invention to provide compositions that modulate Treg responses.

It is another object to provide compositions and methods for treating cancer or tumors.

### SUMMARY OF THE INVENTION

Compositions and methods for increasing IFNγ producing cells and decreasing Treg cells at a tumor site or pathogen infected area in a subject are provided. The compositions can be used to increase frequency and/or percentage of antigen-specific T cells and/or proliferation of antigen-specific T cells, enhance cytokine production by T cells, stimulate differentiation and effector functions of T cells, promote T cell survival, or overcome T cell exhaustion and/or anergy. In a preferred embodiment, the compositions simultaneously block both PD-L1 and PD-L2 mediated signal transduction in T cells, which have differential effects on T cell activity. Blocking PD-L1 mediated signal transduction induces robust effector cell responses, such as increasing the number of infiltrating IFNγ producing T cells and M1 macrophages. Blocking PD-L2 mediated signal transduction decreases the number of infiltrating Tregs. This decrease in Tregs can increase the number of Th17 cells and the level of IL-17 production, and also reduce the number of PD-1 positive cells. Therefore, simultaneous blocking of two independent PD-1 ligands can enhance two different beneficial T cell activities. Preferred compositions include immunomodulatory agents that bind directly to PD-1, PD-L1, PD-L2, or a combination thereof and increase or activate T cell responses, such as T cell proliferation or activation. The compounds bind to and block the interaction of PD-1 ligands expressed on antigen presenting cells (APCs, such as monocytes, macrophages, dendritic cells, epithelial cells etc) with PD-1 on T cells.

The compositions include PD-L2 proteins, fragments, variants or fusions thereof. A preferred composition includes an effective amount of a non-antibody agent such as a PD-L2 fusion protein (B7-DC-Ig) to reduce or overcome lack of sufficient T cell responses, T cell exhaustion, T cell anergy, as well as activation of monocytes, macrophages, dendritic cells and other APCs, or all of these effects in a subject. The compositions also include PD-L1 proteins, fragments, variants or fusions thereof. PD-L2 and PD-L1 polypeptides, fusion proteins, and fragments can inhibit or reduce the inhibitory signal transduction that occurs through PD-1 in T cells by preventing endogenous ligands of PD-1 from interacting with PD-1. Additional preferred compositions include PD-1 or soluble fragments thereof, that bind to ligands of PD-1 and prevent binding to the endogenous PD-1 receptor on T cells. These fragments of PD-1 are also referred to as soluble PD-1 fragments. A preferred embodiment is a PD-1 fusion protein, PD-1-Ig. Other agents include B7.1 or soluble fragments and fusion proteins thereof, that can bind to PD-L1 and prevent binding of PD-L1 to PD-1.

In certain embodiments, the compositions include immunomodulatory agents that: (i) bind to and block PD-1 without inducing inhibitory signal transduction through PD-1 and prevents binding of ligands, such as PD-L1 and PD-L2, thereby preventing activation of the PD-1 mediated inhibitory signal; (ii) bind to ligands of PD-1 and prevent binding to the PD-1 receptor, thereby preventing activation of the PD-1 mediated inhibitory signal, or (iii) combinations of (i) and (ii).

An immune response can be modulated by providing immunomodulatory agents which bind with different affinity (i.e., more or less as required) to PD-L1, PD-L2, PD-1, and combinations thereof by varying the dosage of agent which is administered, by intermittent dosing over a regime, and combinations thereof, that provides for dissociation of agent from the molecule to which it is bound prior to being administered again (similar to what occurs with antigen elicitation using priming and boosting). In some cases it may be particularly desirable to stimulate the immune system, and then remove the stimulation. The affinity of the antagonist for its binding partner can be used to determine the period of time required for dissociation—a higher affinity agent will take longer to dissociate than a lower affinity agent. Agents that bind to either PD-L1, PD-L2, PD-1, and combinations thereof or which bind with different affinities to the same molecule, can also be used to modulate the degree of immunostimulation.

Therapeutic uses of the immunomodulatory agents and nucleic acids encoding the same are provided. The immunomodulatory agents can be used to treat one or more symptoms related to cancer or infectious disease. Additionally, the immunomodulatory agents can be used to stimulate the immune response of immunosuppressed subjects.

Additional embodiments include antibodies that bind to and block either the PD-1 receptor, without causing inhibitory signal transduction, or ligands of the PD-1 receptor, such as PD-L1 and PD-L2, or both ligands, i.e. bispecific agents. The PD-L2 and PD-L1 polypeptides, fusion proteins, and fragments may also activate T cells by binding to another receptor on the T cells or APCs.

Therapeutic uses for the disclosed compositions include the treatment of one or more symptoms of cancer and/or induction of tumor immunity. Exemplary tumor cells that can be treated, include but not limited to, sarcoma, melanoma, lymphoma, leukemia, neuroblastoma, or carcinoma cells.

The compositions increase T cell responses and help overcome T cell exhaustion, T cell anergy, or both, as well as activate monocytes, macrophages, dendritic cells and other APCs induced by infections or cancer. Representative infections that can be treated with the immunomodulatory agents include, but are not limited to, infections caused by a virus, bacterium, parasite, protozoan, or fungus. Exemplary viral infections that can be treated include, but are not limited to, infections caused by hepatitis virus, human immunodeficiency virus (HIV), human T-lymphotrophic virus (HTLV), herpes virus, influenza, Epstein-Barr virus, filovirus, or a human papilloma virus. Other infections that can be treated include those caused by Plasmodium, Mycoplasma, M. tuberculosis, Bacillus anthracis, Staphylococcus, and C. trachomitis.

The compositions can be administered in combination or alternation with a vaccine containing one or more antigens such as viral antigens, bacterial antigens, protozoan antigens, and tumor specific antigens. The compositions can be used as effective adjuvants with vaccines to increase primary immune responses and effector cell responses in subjects. Preferred subjects to be treated have a weakened or compromised immune system, are greater than 65 years old, or are less than 2 years of age.

### BRIEF DESCRIPTION OF THE DRAWINGS

FIG. 1 is a line graph of B7-H1-Ig-APC versus log unlabeled B7-DC-Ig (nM) showing that B7-DC-Ig binds to PD-1 in a PD-1 binding ELISA and inhibits the binding of B7-H1-Ig-APC. APC=allophycocyanin.

FIG. 2A is a line graph of tumor growth (mm³) versus days post tumor inoculation in mice treated with 100 mg/kg of Cytoxan® (CTX) on day ten. Each line in each graph represents one mouse. FIG. 2B is a line graph of tumor growth (mm³) versus days post tumor inoculation in mice treated with 100 mg/kg CTX Day on day 10 followed by bi-weekly B7-DC-Ig (5 mg/kg) administration starting on day 11. Each line in each graph represents one mouse. Black arrow stands for B7-DC-Ig administration. FIG. 2C is a line graph of tumor volume (mm³) versus days post tumor implantation in mice treated with 100 mg/kg CTX (solid circles) or 100 mg/kg CTX and 5 mg/kg B7-DC-Ig (triangles).

FIG. 3 is a schematic diagram of an experimental design showing that administration of 100 mg/kg CTX and 5 mg/kg B7-DC-Ig eradicates tumors in mice. On day zero, mice were subcutaneously injected with 1×10⁵ CT26 tumor cells. On day 10 the mice were injected with 100 mg/ml CTX. The start of B7-DC-Ig 100 ug/mouse twice a week for four weeks was begun on day 11. On day 45, tumors in 75% of the mice treated with B7-DC-Ig were eradicated. The inset is a graph of percent long time survival versus days post inncoluation of mice treated with 100 mg/ml CTX (dashed line) and mice treated with 100 mg/ml CTX and B7-DC-Ig 100 ug/mouse twice a week for four weeks (solid line).

FIG. 4 is a schematic diagram of an experimental design to showing that CTX+B7-DC-Ig treatment results in tumor specific, memory cytotoxic T lymphocytes. The graph shows percent (CD8/IFNγ) positive splenocytes taken from mice treated with 100 mg/mouse CTX and 100 ug/mouse B7-DC-Ig and treated with no peptide (solid circles), 5 ug/ml ovalbumin (OVA) (solid squares), 50 ug/ml OVA (solid triangles), 5 ug/ml AH1, a CT26 specific peptide (solid, inverted triangles), or 500 ug/ml AH1 (solid diamonds).

FIGS. 5A-D are line graphs of tumor growth (mm³) versus days post inncoluation in mice treated with 100 mg/ml CTX (FIG. 5A), 100 mg/ml CTX+30 μg B7-DC-Ig (FIG. 5B), 100 mg CTX+100 μg B7-DC-Ig (FIG. 5C), or 100 mg/ml CTX+300 μg B7-DC-Ig (FIG. 5D).

FIGS. 6A-C are graphs of percent PD-1⁺ of CD8+ T Cells in treated Balb/C mice. Balb/C mice implanted with 1×10⁵ CT26 cells subcutaneously at age of 9 to 11 weeks of age. On Day 9, mice were injected with 100 mg/kg of CTX, IP. Twenty four hours later, on Day 10, mice were treated with 100 ug of B7-DC-Ig. Vehicle injected control (solid circles), CTX alone (solid squares), CTX+B7-DC-Ig (solid triangles) or B7-DC-Ig alone. Mice were continued with B7-DC-Ig injection, 2 times a week. Four mice from other groups were removed from the study on Day 11 (2 days post CTX) (FIG. 6A), Day 16 (7 days post CTX) (FIG. 6B) and Day 22 (13 days post CTX) (FIG. 6C) for T cell analysis.

FIG. 7 is a schematic diagram showing B7-DC-Ig breaking immune suppression by blocking PD-1 and B7-H1 interaction. B7-DC-Ig can interact with PD-1 expressed on exhausted T cells and prevent the binding of B7-H1 expressed on tumor cells or pathogen infected cells. B7-DC-Ig can increase IFNγ producing cells and decrease Treg cells at tumor site or pathogen infected area.

FIG. 8 is a line graph showing the concentration of serum human B7-DC-Ig as a function of time post-dose (hours) in two Cynomolgus monkeys injected with 10 mg/kg B7-DC-Ig by bolus IV injection.

FIG. 9 is a line graph showing the concentration of serum murine B7-DC-Ig (μg/ml) as a function of time post-dose (hours) in mice injected intraperitoneally with 100 μg, 300 μg or 900 μg of murine B7-DC-Ig on day 0.

FIG. 10 is a series of line graphs showing the Cₘₐₓ or Cₘᵢₙ of murine B7-DC-Ig (μg/ml) as a function the number of doses in mice injected intraperitoneally with 100 μg, 300 μg or 900 μg of murine B7-DC-Ig. Cₘₐₓ was measured 6 hours after each dose and Cₘᵢₙ was determined 2-3 days after each dose. Five mice were used for each data point.

### DETAILED DESCRIPTION OF THE INVENTION

### I. Definitions

The term “isolated” is meant to describe a compound of interest (e.g., either a polynucleotide or a polypeptide) that is in an environment different from that in which the compound naturally occurs e.g. separated from its natural milieu such as by concentrating a peptide to a concentration at which it is not found in nature. “Isolated” is meant to include compounds that are within samples that are significantly enriched for the compound of interest and/or in which the compound of interest is partially or significantly purified. “Significantly” means statistically signficantly greater.

As used herein, the term “polypeptide” refers to a chain of amino acids of any length, regardless of modification (e.g., phosphorylation or glycosylation).

As used herein, a “variant” polypeptide contains at least one amino acid sequence alteration as compared to the amino acid sequence of the corresponding wild-type polypeptide.

As used herein, an “amino acid sequence alteration” can be, for example, a substitution, a deletion, or an insertion of one or more amino acids.

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences

As used herein, an “expression control sequence” is a DNA sequence that controls and regulates the transcription and/or translation of another DNA sequence.

As used herein, “operably linked” means incorporated into a genetic construct so that expression control sequences effectively control expression of a coding sequence of interest.

As used herein, a “fragment” of a polypeptide refers to any subset of the polypeptide that is a shorter polypeptide of the full length protein. Generally, fragments will be five or more amino acids in length.

As used herein, “valency” refers to the number of binding sites available per molecule.

As used herein, “conservative” amino acid substitutions are substitutions wherein the substituted amino acid has similar structural or chemical properties.

As used herein, “non-conservative” amino acid substitutions are those in which the charge, hydrophobicity, or bulk of the substituted amino acid is significantly altered.

As used herein, the term “host cell” refers to prokaryotic and eukaryotic cells into which a recombinant expression vector can be introduced.

As used herein, “transformed” and “transfected” encompass the introduction of a nucleic acid (e.g., a vector) into a cell by a number of techniques known in the art.

As used herein, the term “antibody” is meant to include both intact molecules as well as fragments thereof that include the antigen-binding site. These include Fab and F(ab′)₂ fragments which lack the Fc fragment of an intact antibody.

By “immune cell” is meant a cell of hematopoietic origin and that plays a role in the immune response. Immune cells include lymphocytes (e.g., B cells and T cells), natural killer cells, and myeloid cells (e.g., monocytes, macrophages, eosinophils, mast cells, basophils, and granulocytes).

The term ‘T cell” refers to a CD4+ T cell or a CD8+ T cell. The term T cell includes both TH1 cells, TH2 cells and Th17 cells.

The term “T cell cytoxicity” includes any immune response that is mediated by CD8+ T cell activation. Exemplary immune responses include cytokine production, CD8+ T cell proliferation, granzyme or perforin production, and clearance of an infectious agent.

The term “inhibitory signal transduction” refers to signaling through the PD-1 receptor by endogenous PD-L1 or PD-L2, or any other ligand, having the effect of suppressing, or otherwise reducing, T cell responses, whether by reducing T cell proliferation or by any other inhibitory mechanism.

As used herein “maximum plasma concentration” or “Cmax” means the highest observed concentration of a substance (for example, an immunomudulatory agent) in mammalian plasma after administration of the substance to the mammal.

As used herein “Area Under the Curve” or “AUC” is the area under the curve in a plot of the concentration of a substance in plasma against time. AUC can be a measure of the integral of the instantaneous concentrations during a time interval and has the units mass×time/volume, which can also be expressed as molar concentration×time such as nM×day. AUC is typically calculated by the trapezoidal method (e.g., linear, linear-log). AUC is usually given for the time interval zero to infinity, and other time intervals are indicated (for example AUC (t1,t2) where t1 and t2 are the starting and finishing times for the interval). Thus, as used herein “AUC₀₋₂₄ₕ” refers to an AUC over a 24-hour period, and “AUC₀₋₄ₕ” refers to an AUC over a 4-hour period.

As used herein “weighted mean AUC” is the AUC divided by the time interval over which the time AUC is calculated. For instance, weighted mean AUC₀₋₂₄ₕ would represent the AUC₀₋₂₄ₕ divided by 24 hours.

As used herein “confidence interval” or “CI” is an interval in which a measurement or trial falls corresponding to a given probability p where p refers to a 90% or 95% CI and are calculated around either an arithmetic mean, a geometric mean, or a least squares mean. As used herein, a geometric mean is the mean of the natural log-transformed values back-transformed through exponentiation, and the least squares mean may or may not be a geometric mean as well but is derived from the analysis of variance (ANOVA) model using fixed effects.

As used herein the “coefficient of variation (CV)” is a measure of dispersion and it is defined as the ratio of the standard deviation to the mean. It is reported as a percentage (%) by multiplying the above calculation by 100 (% CV).

As used herein “Tmax” refers to the observed time for reaching the maximum concentration of a substance in plasma of a mammal after administration of that substance to the mammal.

As used herein “serum or plasma half life” refers to the time required for half the quantity of a substance administered to a mammal to be metabolized or eliminated from the serum or plasma of the mammal by normal biological processes.

### II. Immunomodulatory Agents

Immune responses can be enhanced using one or more of the immunomodulatory agents described herein. Preferred immunomodulatory agents interfere with or inhibit the interaction between the endogenous ligands of PD-1 and PD-1. For example, the immunomodulatory agent interferes with, inhibits, or blocks PD-L1 (also known as B7-H1), PD-L2 (also known as B7-DC), or both ligands from interacting with PD-1. A preferred immunomodulatory agent interferes with the interaction of both PD-L1 and PD-L2 with PD-1. In some embodiments, the PD-1 ligands are inhibited from binding to PD-1 on T cells, B cells, natural killer (NK) cells, monocytes, dendritic cells or macrophages. In one embodiment, PD-1 ligands are inhibited from binding to PD-1 on activated T cells.

Suitable immunomodulatory agents include, but are not limited to PD-L2, the extracellular domain of PD-L2, fusion proteins of PD-L2, and variants thereof which prevent binding of both PD-L1 and PD-L2 to PD-1. Additional immunomodulatory agents include PD-L1, the extracellular domain of PD-L1, fusion proteins of PD-L1, fragments of PD-L1 and variants thereof which prevent binding of both PD-L1 and PD-L2 to PD-1. In certain embodiments the compositions bind to PD-1 without triggering inhibitory signal transduction through PD-1. PD-1 or soluble fragments thereof that bind to ligands of PD-1 and prevent binding to the endogenous PD-1 receptor on T cells, B7.1 or soluble fragments thereof that can bind to PD-L1 and prevent binding of PD-L1 to PD-1, or combinations of any of the above. In certain embodiments, the immunomodulatory agents increase IFNγ producing cells and decrease Treg cells at a tumor site or pathogen infected area. This decrease in Tregs can increase the number of Th17 cells and the level of IL-17 production, and also reduce the number of PD-1 positive cells. The immunomodulatory agents increase T cell cytotoxicity in a subject, induce a robust immune response in subjects and overcome T cell exhaustion and T cell anergy in the subject.

The immunomodulatory agents bind to ligands of PD-1 and interfere with or inhibit the binding of the ligands to PD-1, or bind directly to PD-1 without engaging in signal transduction through PD-1. In preferred embodiments the immunomodulatory agents bind to ligands of PD-1 and reduce or inhibit the ligands from triggering inhibitory signal transduction through PD-1. In other embodiments, the immunomodulatory agents bind directly to PD-1 and block PD-1 inhibitory signal transduction. In still another embodiment, the immunomodulatory agents can activate T cells by binding to a receptor other than the PD-1 receptor.

The immunomodulatory agents can be small molecule antagonists. The term “small molecule” refers to small organic compounds having a molecular weight of more than 100 and less than about 2,500 daltons, preferably between 100 and 2000, more preferably between about 100 and about 1250, more preferably between about 100 and about 1000, more preferably between about 100 and about 750, more preferably between about 200 and about 500 daltons. The small molecules often include cyclical carbon or heterocyclic structures and/or aromatic or polyaromatic structures substituted with one or more functional groups. The small molecule antagonists reduce or interfere with PD-1 receptor signal transduction by binding to ligands of PD-1 such as PD-L1 and PD-L2 and prevent the ligand from interacting with PD-1 or by binding directly to PD-1 without triggering signal transduction through PD-1.

Additional embodiments include antibodies that bind to PD-L2, PD-L1, PD-1 or B7-1 polypeptides, and variants and/or fragments thereof.

The disclosed immunomodulatory agents preferably bind to PD-1, or a ligand thereof, for a period of less than three months, two months, one month, three weeks, two weeks, one week, or 5 days after in vivo administration to a mammal.

A. PD-L2 Based Immunomodulatory Agents

1. PD-L2 Based Immunomodulatory Agents that Bind to PD-1

In certain embodiments, immunomodulatory agents bind to PD-1 on immune cells and block inhibitory PD-1 signaling by preventing endogenous ligands of PD-1 from interacting with PD-1. PD-1 signal transduction is thought to require binding to PD-1 by a PD-1 ligand (PD-L2 or PD-L1; typically PD-L1) in close proximity to the TCR:MHC complex within the immune synapse. Therefore, proteins, antibodies or small molecules that block inhibitory signal transduction through PD-1 and optionally prevent co-ligation of PD-1 and TCR on the T cell membrane are useful immunomodulatory agents.

Representative polypeptide immunomodulatory agents include, but are not limited to, PD-L2 polypeptides, fragments thereof, fusion proteins thereof, and variants thereof. PD-L2 polypeptides that bind to PD-1 and block inhibitory signal transduction through PD-1 are one of the preferred embodiments. Other embodiments include immunomodulatory agents that prevent native ligands of PD-1 from binding and triggering signal transduction. In certain embodiments, it is believed that the disclosed PD-L2 polypeptides have reduced or no ability to trigger signal transduction through the PD-1 receptor because there is no co-ligation of the TCR by the peptide-MHC complex in the context of the immune synapse. Because signal transduction through the PD-1 receptor transmits a negative signal that attenuates T-cell activation and T-cell proliferation, inhibiting the PD-1 signal transduction pathway allows cells to be activated that would otherwise be attenuated.

2. Exemplary PD-L2 Polypeptide Immunomodulatory Agents

Murine PD-L2 polypeptides can have at least 80%, 85%, 90%, 95%, 99% or 100% sequence identity to:

| (SEQ ID NO: 1) | |
|---|---|
| MLLLLPILNL SLQLHPVAAL FTVTAPKEVY TVDVGSSVSL ECDFDRRECT ELEGIRASLQ | 60 |
| KVENDTSLQS ERATLLEEQL PLGKALFHIP SVQVRDSGQY RCLVICGAAW DYKYLTVKVK | 120 |
| ASYMRIDTRI LEVPGTGEVQ LTCQARGYPL AEVSWQNVSV PANTSHIRTP EGLYQVTSVL | 180 |
| RLKPQPSRNF SCMFWNAHMK ELTSAIIDPL SRMEPKVPRT WPLHVFIPAC TIALIFLAIV | 240 |
| IIQRKRI | 247 |
| or | |
| (SEQ ID NO: 2) | |
| LFTVTAPKEV YTVDVGSSVS LECDFDRREC TELEGIRASL QKVENDTSLQ SERATLLEEQ | 60 |
| LPLGKALFHI PSVQVRDSGQ YRCLVICGAA WDYKYLTVKV KASYMRIDTR ILEVPGTGEV | 120 |
| QLTCQARGYP LAEVSWQNVS VPANTSHIRT PEGLYQVTSV LRLKPQPSRN FSCMFWNAHM | 180 |
| KELTSAIIDP LSRMEPKVPR TWPLHVFIPA CTIALIFLAI VIIQRKRI. | 228 |

Human PD-L2 polypeptides can have at least 80%, 85%, 90%, 95%, 99% or 100% sequence identity to:

| (SEQ ID NO: 3) | |
|---|---|
| MIFLLLMLSL ELQLHQIAAL FTVTVPKELY IIEHGSNVTL ECNFDTGSHV NLGAITASLQ | 60 |
| KVENDTSPHR ERATLLEEQL PLGKASFHIP QVQVRDEGQY QCIIIYGVAW DYKYLTLKVK | 120 |
| ASYRKINTHI LKVPETDEVE LTCQATGYPL AEVSWPNVSV PANTSHSRTP EGLYQVTSVL | 180 |
| RLKPPPGRNF SCVFWNTHVR ELTLASIDLQ SQMEPRTHPT WLLHIFIPFC IIAFIFIATV | 240 |
| IALRKQLCQK LYSSKDTTKR PVTTTKREVN SAI | 273 |
| or | |
| (SEQ ID NO: 4) | |
| LFTVTVPKEL YIIEHGSNVT LECNFDTGSH VNLGAITASL QKVENDTSPH RERATLLEEQ | 60 |
| LPLGKASFHI PQVQVRDEGQ YQCIIIYGVA WDYKYLTLKV KASYRKINTH ILKVPETDEV | 120 |
| ELTCQATGYP LAEVSWPNVS VPANTSHSRT PEGLYQVTSV LRLKPPPGRN FSCVFWNTHV | 180 |
| RELTLASIDL QSQMEPRTHP TWLLHIFIPF CIIAFIFIAT VIALRKQLCQ KLYSSKDTTK | 240 |
| RPVTTTKREV NSAI. | 254 |

Non-human primate (Cynomolgus) PD-L2 polypeptides can have at least 80%, 85%, 90%, 95%, 99% or 100% sequence identity to:

| (SEQ ID NO: 5) | |
|---|---|
| MIFLLLMLSL ELQLHQIAAL FTVTVPKELY IIEHGSNVTL ECNFDTGSHV NLGAITASLQ | 60 |
| KVENDTSPHR ERATLLEEQL PLGKASFHIP QVQVRDEGQY QCIIIYGVAW DYKYLTLKVK | 120 |
| ASYRKINTHI LKVPETDEVE LTCQATGYPL AEVSWPNVSV PANTSHSRTP EGLYQVTSVL | 180 |
| RLKPPPGRNF SCVFWNTHVR ELTLASIDLQ SQMEPRTHPT WLLHIFIPSC IIAFIFIATV | 240 |
| IALRKQLCQK LYSSKDATKR PVTTTKREVN SAI | 273 |
| or | |
| (SEQ ID NO: 6) | |
| LFTVTVPKEL YIIEHGSNVT LECNFDTGSH VNLGAITASL QKVENDTSPH RERATLLEEQ | 60 |
| LPLGKASFHI PQVQVRDEGQ YQCIIIYGVA WDYKYLTLKV KASYRKINTH ILKVPETDEV | 120 |
| ELTCQATGYP LAEVSWPNVS VPANTSHSRT PEGLYQVTSV LRLKPPPGRN FSCVFWNTHV | 180 |
| RELTLASIDL QSQMEPRTHP TWLLHIFIPS CIIAFIFIAT VIALRKQLCQ KLYSSKDATK | 240 |
| RPVTTTKREV NSAI | 254 |

SEQ ID NOs: 1, 3 and 5 each contain a signal peptide.

B. PD-L1 Based Immunomodulatory Agents

1. PD-L1 Based Immunomodulatory Agents that Bind to PD-1 Receptors

Other immunomodulatory agents that bind to the PD-1 receptor include, but are not limited to, PD-L1 polypeptides, fragments thereof, fusion proteins thereof, and variants thereof. These immunomodulatory agents bind to and block the PD-1 receptor and have reduced or no ability to trigger inhibitory signal transduction through the PD-1 receptor. In one embodiment, it is believed that the PD-L1 polypeptides have reduced or no ability to trigger signal transduction through the PD-1 receptor because there is no co-ligation of the TCR by the peptide-MHC complex in the context of the immune synapse. Because signal transduction through the PD-1 receptor transmits a negative signal that attenuates T-cell activation and T-cell proliferation, inhibiting the PD-1 signal transduction using PD-L1 polypeptides allows cells to be activated that would otherwise be attenuated.

2. Exemplary PD-L1 Polypeptide Immunomodulatory Agents

Murine PD-L1 polypeptides can have at least 80%, 85%, 90%, 95%, 99% or 100% sequence identity to:

| (SEQ ID NO: 7) | |
|---|---|
| MRIFAGIIFT ACCHLLRAFT ITAPKDLYVV EYGSNVTMEC RFPVERELDL LALVVYWEKE | 60 |
| DEQVIQFVAG EEDLKPQHSN FRGRASLPKD QLLKGNAALQ ITDVKLQDAG VYCCIISYGG | 120 |
| ADYKRITLKV NAPYRKINQR ISVDPATSEH ELICQAEGYP EAEVIWTNSD HQPVSGKRSV | 180 |
| TTSRTEGMLL NVTSSLRVNA TANDVFYCTF WRSQPGQNHT AELIIPELPA THPPQNRTHW | 240 |
| VLLGSILLFL IVVSTVLLFL RKQVRMLDVE KCGVEDTSSK NRNDTQFEET | 290 |
| or | |
| (SEQ ID NO: 8) | |
| FTITAPKDLY VVEYGSNVTM ECRFPVEREL DLLALVVYWE KEDEQVIQFV AGEEDLKPQH | 60 |
| SNFRGRASLP KDQLLKGNAA LQITDVKLQD AGVYCCIISY GGADYKRITL KVNAPYRKIN | 120 |
| QRISVDPATS EHELICQAEG YPEAEVIWTN SDHQPVSGKR SVTTSRTEGM LLNVTSSLRV | 180 |
| NATANDVFYC TFWRSQPGQN HTAELIIPEL PATHPPQNRT HWVLLGSILL FLIVVSTVLL | 240 |
| FLRKQVRMLD VEKCGVEDTS SKNRNDTQFE ET. | 272 |

Human PD-L1 polypeptides can have at least 80%, 85%, 90%, 95%, 99% or 100% sequence identity to:

| (SEQ ID NO: 9) | |
|---|---|
| MRIFAVFIFM TYWHLLNAFT VTVPKDLYVV EYGSNMTIEC KFPVEKQLDL AALIVYWEME | 60 |
| DKNIIQFVHG EEDLKVQHSS YRQRARLLKD QLSLGNAALQ ITDVKLQDAG VYRCMISYGG | 120 |
| ADYKRITVKV NAPYNKINQR ILVVDPVTSE HELTCQAEGY PKAEVIWTSS DHQVLSGKTT | 180 |
| TTNSKREEKL FNVTSTLRIN TTTNEIFYCT FRRLDPEENH TAELVIPELP LAHPPNERTH | 240 |
| LVILGAILLC LGVALTFIFR LRKGRMMDVK KCGIQDTNSK KQSDTHLEET | 290 |
| or | |
| (SEQ ID NO: 10) | |
| FTVTVPKDLY VVEYGSNMTI ECKFPVEKQL DLAALIVYWE MEDKNIIQFV HGEEDLKVQH | 60 |
| SSYRQRARLL KDQLSLGNAA LQITDVKLQD AGVYRCMISY GGADYKRITV KVNAPYNKIN | 120 |
| QRILVVDPVT SEHELTCQAE GYPKAEVIWT SSDHQVLSGK TTTTNSKREE KLFNVTSTLR | 180 |
| INTTTNEIFY CTFRRLDPEE NHTAELVIPE LPLAHPPNER THLVILGAIL LCLGVALTFI | 240 |
| FRLRKGRMMD VKKCGIQDTN SKKQSDTHLE ET. | 272 |

SEQ ID NOs: 7 and 9 each contain a signal peptide.

C. B7.1 and PD-1 Based Immunomodulatory Agents

1. B7.1 and PD-1 Based Immunomodulatory Agents that Bind to PD-L1 and PD-L2

Other useful polypeptides include the PD-1 receptor protein, or soluble fragments thereof, fusion proteins thereof, and variants thereof, which can bind to the PD-1 ligands, such as PD-L1 or PD-L2, and prevent binding to the endogenous PD-1 receptor, thereby preventing inhibitory signal transduction. Such fragments also include the soluble ECD portion of the PD-1 protein that optionally includes mutations, such as the A99L mutation, that increases binding to the natural ligands. PD-L1 has also been shown to bind the protein B7.1 (Butte, et al., Immunity, 27(1): 111-122 (2007); Butte, et al., Mol. Immunol. 45: 3567-3572 (2008))). Therefore, B7.1 or soluble fragments thereof, which can bind to the PD-L1 ligand and prevent binding to the endogenous PD-1 receptor, thereby preventing inhibitory signal transduction, are also useful.

2. Exemplary B7.1 Polypeptide Immunomodulatory Agents

Murine B7.1 polypeptides can have at least 80%, 85%, 90%, 95%, 99% or 100% sequence identity to:

| | |
|---|---|
| MACNCQLMQD TPLLKFPCPR LILLFVLLIR LSQVSSDVDE QLSKSVKDKV LLPCRYNSPH | 60 |
| EDESEDRIYW QKHDKVVLSV IAGKLKVWPE YKNRTLYDNT TYSLIILGLV LSDRGTYSCV | 120 |

Human B7.1 polypeptides can have at least 80%, 85%, 90%, 95%, 99% or 100% sequence identity to:

| (SEQ ID NO: 13) | |
|---|---|
| MGHTRRQGTS PSKCPYLNFF QLLVLAGLSH FCSGVIHVTK EVKEVATLSC GHNVSVEELA | 60 |
| QTRIYWQKEK KMVLTMMSGD MNIWPEYKNR TIFDITNNLS IVILALRPSD EGTYECVVLK | 120 |
| YEKDAFKREH LAEVTLSVKA DFPTPSISDF EIPTSNIRRI ICSTSGGFPE PHLSWLENGE | 180 |
| ELNAINTTVS QDPETELYAV SSKLDFNMTT NHSFMCLIKY GHLRVNQTFN WNTTKQEHFP | 240 |
| DNLLPSWAIT LISVNGIFVI CCLTYCFAPR CRERRRNERL RRESVRPV | 288 |
| or | |
| (SEQ ID NO: 14) | |
| VIHVTKEVKE VATLSCGHNV SVEELAQTRI YWQKEKKMVL TMMSGDMNIW PEYKNRTIFD | 60 |
| ITNNLSIVIL ALRPSDEGTY ECVVLKYEKD AFKREHLAEV TLSVKADFPT PSISDFEIPT | 120 |
| SNIRRIICST SGGFPEPHLS WLENGEELNA INTTVSQDPE TELYAVSSKL DFNMTTNHSF | 180 |
| MCLIKYGHLR VNQTFNWNTT KQEHFPDNLL PSWAITLISV NGIFVICCLT YCFAPRCRER | 240 |
| RRNERLRRES VRPV. | 254 |

SEQ ID NOs: 11 and 13 each contain a signal peptide.

3. Exemplary PD-1 Polypeptide Immunomodulatory Agents

Human PD-1 polypeptides can have at least 80%, 85%, 90%, 95%, 99% or 100% sequence identity to:

| (SEQ ID NO: 15) | |
|---|---|
| MQIPQAPWPV VWAVLQLGWR PGWFLDSPDR PWNPPTFFPA LLVVTEGDNA TFTCSFSNTS | 60 |
| ESFVLNWYRM SPSNQTDKLA AFPEDRSQPG QDCRFRVTQL PNGRDFHMSV VRARRNDSGT | 120 |
| YLCGAISLAP KAQIKESLRA ELRVTERRAE VPTAHPSPSP RPAGQFQTLV VGVVGGLLGS | 180 |
| LVLLVWVLAV ICSRAARGTI GARRTGQPLK EDPSAVPVFS VDYGELDFQW REKTPEPPVP | 240 |
| CVPEQTEYAT IVFPSGMGTS SPARRGSADG PRSAQPLRPE DGHCSWPL | 288 |

Non-human primate (Cynomolgus) PD-1 polypeptides can have at least 80%, 85%, 90%, 95%, 99% or 100% sequence identity to:

| (SEQ ID NO: 16) | |
|---|---|
| MQIPQAPWPV VWAVLQLGWR PGWFLESPDR PWNAPTFSPA LLLVTEGDNA TFTCSFSNAS | 60 |
| ESFVLNWYRM SPSNQTDKLA AFPEDRSQPG QDCRFRVTRL PNGRDFHMSV VRARRNDSGT | 120 |
| YLCGAISLAP KAQIKESLRA ELRVTERRAE VPTAHPSPSP RPAGQFQTLV VGVVGGLLGS | 180 |
| LVLLVWVLAV ICSRAARGTI GARRTGQPLK EDPSAVPVFS VDYGELDFQW REKTPEPPVP | 240 |
| CVPEQTEYAT IVFPSGMGTS SPARRGSADG PRSAQPLRPE DGHCSWPL | 288 |

Murine PD-1 polypeptides can have at least 80%, 85%, 90%, 95%, 99% or 100% sequence identity to:

| (SEQ ID NO: 17) | |
|---|---|
| MWVRQVPWSF TWAVLQLSWQ SGWLLEVPNG PWRSLTFYPA WLTVSEGANA TFTCSLSNWS | 60 |
| EDLMLNWNRL SPSNQTEKQA AFCNGLSQPV QDARFQIIQL PNRHDFHMNI LDTRRNDSGI | 120 |
| YLCGAISLHP KAKIEESPGA ELVVTERILE TSTRYPSPSP KPEGRFQGMV IGIMSALVGI | 180 |
| PVLLLLAWAL AVFCSTSMSE ARGAGSKDDT LKEEPSAAPV PSVAYEELDF QGREKTPELP | 240 |
| TACVHTEYAT IVFTEGLGAS AMGRRGSADG LQGPRPPRHE DGHCSWPL | 288 |

SEQ ID NOs: 15-17 each contain a signal peptide.

D. Fragments of PD-1 Immunomodulatory Agents

The polypeptide immunomodulatory agents can be full-length polypeptides, or can be a fragment of a full length polypeptide. As used herein, a fragment of a polypeptide immunomodulatory agent refers to any subset of the polypeptide that is a shorter polypeptide of the full length protein.

Useful fragments are those that retain the ability to bind to their natural ligands. A polypeptide immunomodulatory agent that is a fragment of full-length polypeptide typically has at least 20 percent, 30 percent, 40 percent, 50 percent, 60 percent, 70 percent, 80 percent, 90 percent, 95 percent, 98 percent, 99 percent, 100 percent, or even more than 100 percent of the ability to bind its natural ligand(s) as compared to the full-length polypeptide.

For example, useful fragments of PD-L2 and PD-L1 are those that retain the ability to bind to PD-1. PD-L2 and PD-L1 fragments typically have at least 20 percent, 30 percent, 40 percent, 50 percent, 60 percent, 70 percent, 80 percent, 90 percent, 95 percent, 98 percent, 99 percent, 100 percent, or even more than 100 percent of the ability to bind to PD-1 as compared to full length PD-L2 and PD-L1.

Fragments of polypeptide immunomodulatory agents include soluble fragments. Soluble polypeptide immunomodulatory agent fragments are fragments of polypeptides that may be shed, secreted or otherwise extracted from the producing cells. Soluble fragments of polypeptide immunomodulatory agents include some or all of the extracellular domain of the polypeptide, and lack some or all of the intracellular and/or transmembrane domains. In one embodiment, polypeptide immunomodulatory agent fragments include the entire extracellular domain of the immunomodulatory polypeptide. It will be appreciated that the extracellular domain can include 1, 2, 3, 4, or 5 amino acids from the transmembrane domain. Alternatively, the extracellular domain can have 1, 2, 3, 4, or 5 amino acids removed from the C-terminus, N-terminus, or both.

Generally, the immunomodulatory polypeptides or fragments thereof are expressed from nucleic acids that include sequences that encode a signal sequence. The signal sequence is generally cleaved from the immature polypeptide to produce the mature polypeptide lacking the signal sequence. The signal sequence of immunomodulatory polypeptides can be replaced by the signal sequence of another polypeptide using standard molecule biology techniques to affect the expression levels, secretion, solubility, or other property of the polypeptide. The signal sequence that is used to replace the immunomodulatory polypeptide signal sequence can be any known in the art.

1. PD-L2 Extracellular Domains

a. Human PD-L2 Extracellular Domains

In one embodiment, the immunomodulatory polypeptide includes the extracellular domain of human PD-L2 or a fragment thereof. The immunomodulatory polypeptide can be encoded by a nucleotide sequence having at least 80%, 85%, 90%, 95%, 99%, or 100% sequence identity to:

| (SEQ ID NO: 18) | |
|---|---|
| atgatctttc ttctcttgat gctgtctttg gaattgcaac ttcaccaaat cgcggccctc | 60 |
| tttactgtga ccgtgccaaa agaactgtat atcattgagc acgggtccaa tgtgaccctc | 120 |
| gaatgtaact ttgacaccgg cagccacgtt aacctggggg ccatcactgc cagcttgcaa | 180 |
| aaagttgaaa acgacacttc acctcaccgg gagagggcaa ccctcttgga ggagcaactg | 240 |
| ccattgggga aggcctcctt tcatatccct caggtgcagg ttcgggatga gggacagtac | 300 |
| cagtgcatta ttatctacgg cgtggcttgg gattacaagt atctgaccct gaaggtgaaa | 360 |
| gcgtcctatc ggaaaattaa cactcacatt cttaaggtgc cagagacgga cgaggtggaa | 420 |
| ctgacatgcc aagccaccgg ctacccgttg gcagaggtca gctggcccaa cgtgagcgta | 480 |
| cctgctaaca cttctcattc taggacaccc gagggcctct accaggttac atccgtgctc | 540 |
| cgcctcaaac cgcccccagg ccggaatttt agttgcgtgt tttggaatac ccacgtgcga | 600 |
| gagctgactc ttgcatctat tgatctgcag tcccagatgg agccacggac tcatccaact | 660 |
| tgg. | 663 |

In another embodiment, the immunomodulatory polypeptide can have at least 80%, 85%, 90%, 95%, 99%, or 100% sequence identity to the human amino acid sequence:

| (SEQ ID NO: 19) | |
|---|---|
| MIFLLLMLSL ELQLHQIAAL FTVTVPKELY IIEHGSNVTL MIFLLLMLSL ELQLHQIAAL | 60 |
| FTVTVPKELY IIEHGSNVTL ECNFDTGSHV NLGAITASLQ KVENDTSPHR ERATLLEEQL | 120 |
| PLGKASFHIP QVQVRDEGQY QCIIIYGVAW DYKYLTLKVK ASYRKINTHI LKVPETDEVE | 180 |
| LTCQATGYPL AEVSWPNVSV PANTSHSRTP EGLYQVTSVL RLKPPPGRNF SCVFWNTHVR | 240 |
| ELTLASIDLQ SQMEPRTHPT W. | 261 |

It will be appreciated that the signal sequence will be removed in the mature protein. Additionally, it will be appreciated that signal peptides from other organisms can be used to enhance the secretion of the protein from a host during manufacture. SEQ ID NO:20 provides the human amino acid sequence of SEQ ID NO:19 without the signal sequence:

| (SEQ ID NO: 20) | |
|---|---|
| LFTVTVPKEL YIIEHGSNVT LECNFDTGSH VNLGAITASL QKVENDTSPH RERATLLEEQ | 60 |
| LPLGKASFHI PQVQVRDEGQ YQCIIIYGVA WDYKYLTLKV KASYRKINTH ILKVPETDEV | 120 |
| ELTCQATGYP LAEVSWPNVS VPANTSHSRT PEGLYQVTSV LRLKPPPGRN FSCVFWNTHV | 180 |
| RELTLASIDL QSQMEPRTHP TW. | 202 |

In another embodiment, the immunomodulatory polypeptide includes the IgV domain of human PD-L2. The polypeptide can be encoded by a nucleotide sequence having at least 80%, 85%, 90%, 95%, 99%, or 100% sequence identity to:

| (SEQ ID NO: 21) | |
|---|---|
| tttactgtga ccgtgccaaa agaactgtat atcattgagc acgggtccaa tgtgaccctc | 60 |
| gaatgtaact ttgacaccgg cagccacgtt aacctggggg ccatcactgc cagcttgcaa | 120 |
| aaagttgaaa acgacacttc acctcaccgg gagagggcaa ccctcttgga ggagcaactg | 180 |
| ccattgggga aggcctcctt tcatatccct caggtgcagg ttcgggatga gggacagtac | 240 |
| cagtgcatta ttatctacgg cgtggcttgg gattacaagt atctgaccct gaag. | 294 |

The immunomodulatory polypeptide can have at least 80%, 85%, 90%, 95%, 99%, or 100% sequence identity to the human amino acid sequence:

| (SEQ ID NO: 22) | |
|---|---|
| FTVTVPKELY IIEHGSNVTL ECNFDTGSHV NLGAITASLQ KVENDTSPHR ERATLLEEQL | 60 |
| PLGKASFHIP QVQVRDEGQY QCIIIYGVAW DYKYLTLK,. | 98 |
| also referred to as PD-L2V | |

b. Non-Human Primate PD-L2 Extracellular Domains

In one embodiment, the immunomodulatory polypeptide includes the extracellular domain of non-human primate (Cynomolgus) PD-L2 or a fragment thereof. The polypeptide can be encoded by a nucleotide sequence having at least 80%, 85%, 90%, 95%, 99%, or 100% sequence identity to:

| (SEQ ID NO: 23) | |
|---|---|
| atgatcttcc tcctgctaat gttgagcctg gaattgcagc ttcaccagat agcagcttta | 60 |
| ttcacagtga cagtccctaa ggaactgtac ataatagagc atggcagcaa tgtgaccctg | 120 |
| gaatgcaact ttgacactgg aagtcatgtg aaccttggag caataacagc cagtttgcaa | 180 |
| aaggtggaaa atgatacatc cccacaccgt gaaagagcca ctttgctgga ggagcagctg | 240 |
| cccctaggga aggcctcgtt ccacatacct caagtccaag tgagggacga aggacagtac | 300 |
| caatgcataa tcatctatgg ggtcgcctgg gactacaagt acctgactct gaaagtcaaa | 360 |
| gcttcctaca ggaaaataaa cactcacatc ctaaaggttc cagaaacaga tgaggtagag | 420 |
| ctcacctgcc aggctacagg ttatcctctg gcagaagtat cctggccaaa cgtcagcgtt | 480 |
| cctgccaaca ccagccactc caggacccct gaaggcctct accaggtcac cagtgttctg | 540 |
| cgcctaaagc caccccctgg cagaaacttc agctgtgtgt tctggaatac tcacgtgagg | 600 |
| gaacttactt tggccagcat tgaccttcaa agtcagatgg aacccaggac ccatccaact | 660 |
| tgg. | 663 |

In another embodiment, the immunomodulatory polypeptide can have at least 80%, 85%, 90%, 95%, 99%, or 100% sequence identity to the non-human primate amino acid sequence:

| (SEQ ID NO: 24) | |
|---|---|
| MIFLLLMLSL ELQLHQIAAL FTVTVPKELY IIEHGSNVTL ECNFDTGSHV NLGAITASLQ | 60 |
| KVENDTSPHR ERATLLEEQL PLGKASFHIP QVQVRDEGQY QCIIIYGVAW DYKYLTLKVK | 120 |
| ASYRKINTHI LKVPETDEVE LTCQATGYPL AEVSWPNVSV PANTSHSRTP EGLYQVTSVL | 180 |
| RLKPPPGRNF SCVFWNTHVR ELTLASIDLQ SQMEPRTHPT W. | 221 |

The signal sequence will be removed in the mature protein. Additionally, signal peptides from other organisms can be used to enhance the secretion of the protein from a host during manufacture. SEQ ID NO:25 provides the non-human primate amino acid sequence of SEQ ID NO:24 without the signal sequence:

| (SEQ ID NO: 25) | |
|---|---|
| LFTVTVPKEL YIIEHGSNVT LECNFDTGSH VNLGAITASL QKVENDTSPH RERATLLEEQ | 60 |
| LPLGKASFHI PQVQVRDEGQ YQCIIIYGVA WDYKYLTLKV KASYRKINTH ILKVPETDEV | 120 |
| ELTCQATGYP LAEVSWPNVS VPANTSHSRT PEGLYQVTSV LRLKPPPGRN FSCVFWNTHV | 180 |
| RELTLASIDL QSQMEPRTHP TW. | 202 |

In another embodiment, the immunomodulatory polypeptide includes the IgV domain of non-human primate PD-L2. The polypeptide can be encoded by a nucleotide sequence having at least 80%, 85%, 90%, 95%, 99%, or 100% sequence identity to:

| (SEQ ID NO: 26) | |
|---|---|
| ttcacagtga cagtccctaa ggaactgtac ataatagagc atggcagcaa tgtgaccctg | 60 |
| gaatgcaact ttgacactgg aagtcatgtg aaccttggag caataacagc cagtttgcaa | 120 |
| aaggtggaaa atgatacatc cccacaccgt gaaagagcca ctttgctgga ggagcagctg | 180 |
| cccctaggga aggcctcgtt ccacatacct caagtccaag tgagggacga aggacagtac | 240 |
| caatgcataa tcatctatgg ggtcgcctgg gactacaagt acctgactct gaaa. | 294 |

The immunomodulatory polypeptide can have at least 80%, 85%, 90%, 95%, 99%, or 100% sequence identity to the non-human primate amino acid sequence:

| (SEQ ID NO: 27) | |
|---|---|
| FTVTVPKELY IIEHGSNVTL ECNFDTGSHV NLGAITASLQ KVENDTSPHR ERATLLEEQL | 60 |
| PLGKASFHIP QVQVRDEGQY QCIIIYGVAW DYKYLTLK, | 98 |
| also referred to as PD-L2V. | |

c. Murine PD-L2 Extracellular Domains

In one embodiment, the immunomodulatory polypeptide includes the extracellular domain of murine PD-L2 or a fragment thereof. The immunomodulatory polypeptide can be encoded by a nucleotide sequence having at least 80%, 85%, 90%, 95%, 99%, or 100% sequence identity to:

| (SEQ ID NO: 28) | |
|---|---|
| atgctgctcc tgctgccgat actgaacctg agcttacaac ttcatcctgt agcagcttta | 60 |
| ttcaccgtga cagcccctaa agaagtgtac accgtagacg tcggcagcag tgtgagcctg | 120 |
| gagtgcgatt ttgaccgcag agaatgcact gaactggaag ggataagagc cagtttgcag | 180 |
| aaggtagaaa atgatacgtc tctgcaaagt gaaagagcca ccctgctgga ggagcagctg | 240 |
| cccctgggaa aggctttgtt ccacatccct agtgtccaag tgagagattc cgggcagtac | 300 |
| cgttgcctgg tcatctgcgg ggccgcctgg gactacaagt acctgacggt gaaagtcaaa | 360 |
| gcttcttaca tgaggataga cactaggatc ctggaggttc caggtacagg ggaggtgcag | 420 |
| cttacctgcc aggctagagg ttatccccta gcagaagtgt cctggcaaaa tgtcagtgtt | 480 |
| cctgccaaca ccagccacat caggaccccc gaaggcctct accaggtcac cagtgttctg | 540 |
| cgcctcaagc ctcagcctag cagaaacttc agctgcatgt tctggaatgc tcacatgaag | 600 |
| gagctgactt cagccatcat tgaccctctg agtcggatgg aacccaaagt ccccagaacg | 660 |
| tgg. | 663 |

In another embodiment, the immunomodulatory polypeptide can have at least 80%, 85%, 90%, 95%, 99%, or 100% sequence identity to the murine amino acid sequence:

| (SEQ ID NO: 29) | |
|---|---|
| MLLLLPILNL SLQLHPVAAL FTVTAPKEVY TVDVGSSVSL ECDFDRRECT ELEGIRASLQ | 60 |
| KVENDTSLQS ERATLLEEQL PLGKALFHIP SVQVRDSGQY RCLVICGAAW DYKYLTVKVK | 120 |
| ASYMRIDTRI LEVPGTGEVQ LTCQARGYPL AEVSWQNVSV PANTSHIRTP EGLYQVTSVL | 180 |
| RLKPQPSRNF SCMFWNAHMK ELTSAIIDPL SRMEPKVPRT W. | 221 |

The signal sequence will be removed in the mature protein. Additionally, signal peptides from other organisms can be used to enhance the secretion of the protein from a host during manufacture. SEQ ID NO:30 provides the murine amino acid sequence of SEQ ID NO:29 without the signal sequence:

| (SEQ ID NO: 30) | |
|---|---|
| LFTVTAPKEV YTVDVGSSVS LECDFDRREC TELEGIRASL QKVENDTSLQ SERATLLEEQ | 60 |
| LPLGKALFHI PSVQVRDSGQ YRCLVICGAA WDYKYLTVKV KASYMRIDTR ILEVPGTGEV | 120 |
| QLTCQARGYP LAEVSWQNVS VPANTSHIRT PEGLYQVTSV LRLKPQPSRN FSCMFWNAHM | 180 |
| KELTSAIIDP LSRMEPKVPR TW. | 202 |

In another embodiment, the immunomodulatory polypeptide includes the IgV domain of murine PD-L2. The polypeptide can be encoded by a nucleotide sequence having at least 80%, 85%, 90%, 95%, 99%, or 100% sequence identity to:

| (SEQ ID NO: 31) | |
|---|---|
| ttcaccgtga cagcccctaa agaagtgtac accgtagacg tcggcagcag tgtgagcctg | 60 |
| gagtgcgatt ttgaccgcag agaatgcact gaactggaag ggataagagc cagtttgcag | 120 |
| aaggtagaaa atgatacgtc tctgcaaagt gaaagagcca ccctgctgga ggagcagctg | 180 |
| cccctgggaa aggctttgtt ccacatccct agtgtccaag tgagagattc cgggcagtac | 240 |
| cgttgcctgg tcatctgcgg ggccgcctgg gactacaagt acctgacggt gaaa | 294 |

The immunomodulatory polypeptide can have at least 80%, 85%, 90%, 95%, 99%, or 100% sequence identity to the murine amino acid sequence:

| (SEQ ID NO: 32) | |
|---|---|
| FTVTAPKEVY TVDVGSSVSL ECDFDRRECT ELEGIRASLQ KVENDTSLQS ERATLLEEQL | 60 |
| PLGKALFHIP SVQVRDSGQY RCLVICGAAW DYKYLTVK, | 98 |
| also referred to as PD-L2V. | |

d. PD-L2 Extracellular Domain Fragments

The PD-L2 extracellular domain can contain one or more amino acids from the signal peptide or the putative transmembrane domain of PD-L2. During secretion, the number of amino acids of the signal peptide that are cleaved can vary depending on the expression system and the host. Additionally, fragments of PD-L2 extracellular domain missing one or more amino acids from the C-terminus or the N-terminus that retain the ability to bind to PD-1 can be used.

Exemplary suitable fragments of murine PD-L2 that can be used include, but are not limited to, the following:

24-221, 24-220, 24-219, 24-218, 24-217, 24-216, 24-215,

23-221, 23-220, 23-219, 23-218, 23-217, 23-216, 23-215,

22-221, 22-220, 22-219, 22-218, 22-217, 22-216, 22-215,

21-221, 21-220, 21-219, 21-218, 21-217, 21-216, 21-215,

20-221, 20-220, 20-219, 20-218, 20-217, 20-216, 20-215,

19-221, 19-220, 19-219, 19-218, 19-217, 19-216, 19-215,

18-221, 18-220, 18-219, 18-218, 18-217, 18-216, 18-215,

17-221, 17-220, 17-219, 17-218, 17-217, 17-216, 17-215,

16-221, 16-220, 16-219, 16-218, 16-217, 16-216, 16-215,

### of SEQ ID NO:56.

Additional suitable fragments of murine PD-L2 include, but are not limited to, the following:

20-221, 33-222, 33-223, 33-224, 33-225, 33-226, 33-227,

21-221, 21-222, 21-223, 21-224, 21-225, 21-226, 21-227,

22-221, 22-222, 22-223, 22-224, 22-225, 22-226, 22-227,

23-221, 23-222, 23-223, 23-224, 23-225, 23-226, 23-227,

24-221, 24-222, 24-223, 24-224, 24-225, 24-226, 24-227,

of SEQ ID NO:1, optionally with one to five amino acids of a signal peptide attached to the N-terminal end. The signal peptide may be any disclosed herein, including the signal peptide contained within SEQ ID NO:1, or may be any signal peptide known in the art.

Exemplary suitable fragments of human PD-L2 that can be used include, but are not limited to, the following:

24-221, 24-220, 24-219, 24-218, 24-217, 24-216, 24-215,

23-221, 23-220, 23-219, 23-218, 23-217, 23-216, 23-215,

22-221, 22-220, 22-219, 22-218, 22-217, 22-216, 22-215,

21-221, 21-220, 21-219, 21-218, 21-217, 21-216, 21-215,

20-221, 20-220, 20-219, 20-218, 20-217, 20-216, 20-215,

19-221, 19-220, 19-219, 19-218, 19-217, 19-216, 19-215,

18-221, 18-220, 18-219, 18-218, 18-217, 18-216, 18-215,

17-221, 17-220, 17-219, 17-218, 17-217, 17-216, 17-215,

16-221, 16-220, 16-219, 16-218, 16-217, 16-216, 16-215,

### of SEQ ID NO:60.

Additional suitable fragments of human PD-L2 include, but are not limited to, the following:

20-221, 20-222, 20-223, 20-224, 20-225, 20-226, 20-227,

21-221, 21-222, 21-223, 21-224, 21-225, 21-226, 21-227,

22-221, 22-222, 22-223, 22-224, 22-225, 22-226, 22-227,

23-221, 23-222, 23-223, 23-224, 23-225, 23-226, 23-227,

24-221, 24-222, 24-223, 24-224, 24-225, 24-226, 24-227,

of SEQ ID NO:3, optionally with one to five amino acids of a signal peptide attached to the N-terminal end. The signal peptide may be any disclosed herein, including the signal peptide contained within SEQ ID NO:3, or may be any signal peptide known in the art.

Exemplary suitable fragments of non-human primate PD-L2 that can be used include, but are not limited to, the following:

24-221, 24-220, 24-219, 24-218, 24-217, 24-216, 24-215,

23-221, 23-220, 23-219, 23-218, 23-217, 23-216, 23-215,

22-221, 22-220, 22-219, 22-218, 22-217, 22-216, 22-215,

21-221, 21-220, 21-219, 21-218, 21-217, 21-216, 21-215,

20-221, 20-220, 20-219, 20-218, 20-217, 20-216, 20-215,

19-221, 19-220, 19-219, 19-218, 19-217, 19-216, 19-215,

18-221, 18-220, 18-219, 18-218, 18-217, 18-216, 18-215,

17-221, 17-220, 17-219, 17-218, 17-217, 17-216, 17-215,

16-221, 16-220, 16-219, 16-218, 16-217, 16-216, 16-215,

### of SEQ ID NO:5.

Additional suitable fragments of non-human primate PD-L2 include, but are not limited to, the following:

20-221, 33-222, 33-223, 33-224, 33-225, 33-226, 33-227,

21-221, 21-222, 21-223, 21-224, 21-225, 21-226, 21-227,

22-221, 22-222, 22-223, 22-224, 22-225, 22-226, 22-227,

23-221, 23-222, 23-223, 23-224, 23-225, 23-226, 23-227,

24-221, 24-222, 24-223, 24-224, 24-225, 24-226, 24-227,

of SEQ ID NO:5, optionally with one to five amino acids of a signal peptide attached to the N-terminal end. The signal peptide may be any disclosed herein, including the signal peptide contained within SEQ ID NO:5, or may be any signal peptide known in the art.

PD-L2 proteins also include a PD-1 binding fragment of amino acids 20-121 of SEQ ID NO:3 (human full length), or amino acids 1-102 of SEQ ID NO:24 (extracellular domain or ECD). In specific embodiments thereof, the PD-L2 polypeptide or PD-1 binding fragment also incorporates amino acids WDYKY at residues 110-114 of SEQ ID NO:3 or WDYKY at residues 91-95 of SEQ ID NO:24. By way of non-limiting examples, such a PD-1 binding fragment comprises at least 10, at least 20, at least 30, at least 40, at least 50, at least 60, at least 70, at least 75, at least 80, at least 85, at least 90, at least 95, or at least 100 contiguous amino acids of the sequence of amino acids 20-121 of SEQ ID NO:3, wherein a preferred embodiment of each such PD-1 binding fragment would comprise as a sub-fragment the amino acids WDYKY found at residues 110-114 of SEQ ID NO:3 or WDYKY at residues 91-95 of SEQ ID NO:24.

2. PD-L1 Extracellular Domains

In one embodiment, the variant PD-L1 polypeptide includes all or part of the extracellular domain. The amino acid sequence of a representative extracellular domain of human PD-L1 can have 80%, 85%, 90%, 95%, or 99% sequence identity to

| (SEQ ID NO: 33) | |
|---|---|
| FTVTVPKDLY VVEYGSNMTI ECKFPVEKQL DLAALIVYWE MEDKNIIQFV HGEEDLKVQH | 60 |
| SSYRQRARLL KDQLSLGNAA LQITDVKLQD AGVYRCMISY GGADYKRITV KVNAPYNKIN | 120 |
| QRILVVDPVT SEHELTCQAE GYPKAEVIWT SSDHQVLSGK TTTTNSKREE KLFNVTSTLR | 180 |
| INTTTNEIFY CTFRRLDPEE NHTAELVIPE LPLAHPPNER. | 220 |

The transmembrane domain of PD-L1 begins at amino acid position 239 of SEQ ID NO:9. It will be appreciated that the suitable fragments of PD-L1 can include 1, 2, 3, 4, 5, 6, 7, 8, 9, or 10 contiguous amino acids of a signal peptide sequence, for example SEQ ID NO:9 or variants thereof, 1, 2, 3, 4, 5, 6, 7, 8, 9, or 10 amino acids of the transmembrane domain, or combinations thereof.

The extracellular domain of murine PD-L1 has the following amino acid sequence

| (SEQ ID NO: 34) | |
|---|---|
| FTITAPKDLY VVEYGSNVTM ECRFPVEREL DLLALVVYWE KEDEQVIQFV AGEEDLKPQH | 60 |
| SNFRGRASLP KDQLLKGNAA LQITDVKLQD AGVYCCIISY GGADYKRITL KVNAPYRKIN | 120 |
| QRISVDPATS EHELICQAEG YPEAEVIWTN SDHQPVSGKR SVTTSRTEGM LLNVTSSLRV | 180 |
| NATANDVFYC TFWRSQPGQN HTAELIIPEL PATHPPQNRT HWVLLGSILL FLIVVSTVL. | 239 |

The transmembrane domain of the murine PD-L1 begins at amino acid position 240 of SEQ ID NO:7. In certain embodiments the PD-L1 polypeptide includes the extracellular domain of murine PD-L1 with 1, 2, 3, 4, 5, 6, 7, 8, 9, or 10 contiguous amino acids of a signal peptide, 1, 2, 3, 4, 5, 6, 7, 8, 9, or 10 contiguous amino acids of the transmembrane domain, or combinations thereof.

3. B7.1 Extracellular Domains

a. Murine B7.1 extracellular domains

In one embodiment, the immunomodulatory polypeptide includes the extracellular domain of murine B7.1 or a fragment thereof. The immunomodulatory polypeptide can be encoded by a nucleotide sequence having at least 80%, 85%, 90%, 95%, 99%, or 100% sequence identity to:

| (SEQ ID NO: 35) | |
|---|---|
| atggcttgca attgtcagtt gatgcaggat acaccactcc tcaagtttcc atgtccaagg | 60 |
| ctcattcttc tctttgtgct gctgattcgt ctttcacaag tgtcttcaga tgttgatgaa | 120 |
| caactgtcca agtcagtgaa agataaggta ttgctgcctt gccgttacaa ctctcctcat | 180 |
| gaagatgagt ctgaagaccg aatctactgg caaaaacatg acaaagtggt gctgtctgtc | 240 |
| attgctggga aactaaaagt gtggcccgag tataagaacc ggactttata tgacaacact | 300 |
| acctactctc ttatcatcct gggcctggtc ctttcagacc ggggcacata cagctgtgtc | 360 |
| gttcaaaaga aggaaagagg aacgtatgaa gttaaacact tggctttagt aaagttgtcc | 420 |
| atcaaagctg acttctctac ccccaacata actgagtctg gaaacccatc tgcagacact | 480 |
| aaaaggatta cctgctttgc ttccgggggt ttcccaaagc ctcgcttctc ttggttggaa | 540 |
| aatggaagag aattacctgg catcaatacg acaatttccc aggatcctga atctgaattg | 600 |
| tacaccatta gtagccaact agatttcaat acgactcgca accacaccat taagtgtctc | 660 |
| attaaatatg gagatgctca cgtgtcagag gacttcacct gggaaaaacc cccagaagac | 720 |
| cctcctgata gcaagaac. | 738 |

In another embodiment, the immunomodulatory polypeptide can have at least 80%, 85%, 90%, 95%, 99%, or 100% sequence identity to the murine amino acid sequence:

| (SEQ ID NO: 36) | |
|---|---|
| MACNCQLMQD TPLLKFPCPR LILLFVLLIR LSQVSSDVDE QLSKSVKDKV LLPCRYNSPH | 60 |
| EDESEDRIYW QKHDKVVLSV IAGKLKVWPE YKNRTLYDNT TYSLIILGLV LSDRGTYSCV | 120 |
| VQKKERGTYE VKHLALVKLS IKADFSTPNI TESGNPSADT KRITCFASGG FPKPRFSWLE | 180 |
| NGRELPGINT TISQDPESEL YTISSQLDFN TTRNHTIKCL IKYGDAHVSE DFTWEKPPED | 240 |
| PPDSKN. | 246 |

The signal sequence will be removed in the mature protein. Additionally, signal peptides from other organisms can be used to enhance the secretion of the protein from a host during manufacture. SEQ ID NO:37 provides the murine amino acid sequence of SEQ ID NO:36 without the signal sequence:

| (SEQ ID NO: 37) | |
|---|---|
| VDEQLSKSVK DKVLLPCRYN SPHEDESEDR IYWQKHDKVV LSVIAGKLKV WPEYKNRTLY | 60 |
| DNTTYSLIIL GLVLSDRGTY SCVVQKKERG TYEVKHLALV KLSIKADFST PNITESGNPS | 120 |
| ADTKRITCFA SGGFPKPRFS WLENGRELPG INTTISQDPE SELYTISSQL DFNTTRNHTI | 180 |
| KCLIKYGDAH VSEDFTWEKP PEDPPDSKN. | 209 |

In another embodiment, the immunomodulatory polypeptide includes the IgV domain of murine B7.1. The polypeptide can be encoded by a nucleotide sequence having at least 80%, 85%, 90%, 95%, 99%, or 100% sequence identity to:

| (SEQ ID NO: 38) | |
|---|---|
| gttgatgaac aactgtccaa gtcagtgaaa gataaggtat tgctgccttg ccgttacaac | 60 |
| tctcctcatg aagatgagtc tgaagaccga atctactggc aaaaacatga caaagtggtg | 120 |
| ctgtctgtca ttgctgggaa actaaaagtg tggcccgagt ataagaaccg gactttatat | 180 |
| gacaacacta cctactctct tatcatcctg ggcctggtcc tttcagaccg gggcacatac | 240 |
| agctgtgtcg ttcaaaagaa ggaaagagga acgtatgaag ttaaacactt g. | 291 |

The immunomodulatory polypeptide can have at least 80%, 85%, 90%, 95%, 99%, or 100% sequence identity to the murine amino acid sequence:

| (SEQ ID NO: 39) | |
|---|---|
| VDEQLSKSVK DKVLLPCRYN SPHEDESEDR IYWQKHDKVV LSVIAGKLKV WPEYKNRTLY | 60 |
| DNTTYSLIIL GLVLSDRGTY SCVVQKKERG TYEVKHL, | 97 |
| also referred to as B7.1V. | |

b. Human B7.1 Extracellular Domains

In one embodiment, the immunomodulatory polypeptide includes the extracellular domain of human B7.1 or a fragment thereof. The immunomodulatory polypeptide can be encoded by a nucleotide sequence having at least 80%, 85%, 90%, 95%, 99%, or 100% sequence identity to:

| (SEQ ID NO: 40) | |
|---|---|
| atgggccaca cacggaggca gggaacatca ccatccaagt gtccatacct caatttcttt | 60 |
| cagctcttgg tgctggctgg tctttctcac ttctgttcag gtgttatcca cgtgaccaag | 120 |
| gaagtgaaag aagtggcaac gctgtcctgt ggtcacaatg tttctgttga agagctggca | 180 |
| caaactcgca tctactggca aaaggagaag aaaatggtgc tgactatgat gtctggggac | 240 |
| atgaatatat ggcccgagta caagaaccgg accatctttg atatcactaa taacctctcc | 300 |
| attgtgatcc tggctctgcg cccatctgac gagggcacat acgagtgtgt tgttctgaag | 360 |
| tatgaaaaag acgctttcaa gcgggaacac ctggctgaag tgacgttatc agtcaaagct | 420 |
| gacttcccta cacctagtat atctgacttt gaaattccaa cttctaatat tagaaggata | 480 |
| atttgctcaa cctctggagg ttttccagag cctcacctct cctggttgga aaatggagaa | 540 |
| gaattaaatg ccatcaacac aacagtttcc caagatcctg aaactgagct ctatgctgtt | 600 |
| agcagcaaac tggatttcaa tatgacaacc aaccacagct tcatgtgtct catcaagtat | 660 |
| ggacatttaa gagtgaatca gaccttcaac tggaatacaa ccaagcaaga gcattttcct | 720 |
| gataacctgc tc. | 732 |

In another embodiment, the immunomodulatory polypeptide can have at least 80%, 85%, 90%, 95%, 99%, or 100% sequence identity to the human amino acid sequence:

| (SEQ ID NO: 41) | |
|---|---|
| MGHTRRQGTS PSKCPYLNFF QLLVLAGLSH FCSGVIHVTK EVKEVATLSC GHNVSVEELA | 60 |
| QTRIYWQKEK KMVLTMMSGD MNIWPEYKNR TIFDITNNLS IVILALRPSD EGTYECVVLK | 120 |
| YEKDAFKREH LAEVTLSVKA DFPTPSISDF EIPTSNIRRI ICSTSGGFPE PHLSWLENGE | 180 |
| ELNAINTTVS QDPETELYAV SSKLDFNMTT NHSFMCLIKY GHLRVNQTFN WNTTKQEHFP | 240 |
| DNL. | 243 |

The signal sequence will be removed in the mature protein. Additionally, signal peptides from other organisms can be used to enhance the secretion of the protein from a host during manufacture. SEQ ID NO:41 provides the human amino acid sequence of SEQ ID NO:40 without the signal sequence:

| (SEQ ID NO: 42) | |
|---|---|
| VIHVTKEVKE VATLSCGHNV SVEELAQTRI YWQKEKKMVL TMMSGDMNIW PEYKNRTIFD | 60 |
| ITNNLSIVIL ALRPSDEGTY ECVVLKYEKD AFKREHLAEV TLSVKADFPT PSISDFEIPT | 120 |
| SNIRRIICST SGGFPEPHLS WLENGEELNA INTTVSQDPE TELYAVSSKL DFNMTTNHSF | 180 |
| MCLIKYGHLR VNQTFNWNTT KQEHFPDNL. | 209 |

In another embodiment, the immunomodulatory polypeptide can have at least 80%, 85%, 90%, 95%, 99%, or 100% sequence identity to SEQ ID NO:41 or SEQ ID NO:42 lacking between 1 and 10 C-terminal amino acids.

In another embodiment, the immunomodulatory polypeptide includes the IgV domain of human B7.1. The polypeptide can be encoded by a nucleotide sequence having at least 80%, 85%, 90%, 95%, 99%, or 100% sequence identity to:

| (SEQ ID NO: 43) | |
|---|---|
| gttatccacg tgaccaagga agtgaaagaa gtggcaacgc tgtcctgtgg tcacaatgtt | 60 |
| tctgttgaag agctggcaca aactcgcatc tactggcaaa aggagaagaa aatggtgctg | 120 |
| actatgatgt ctggggacat gaatatatgg cccgagtaca agaaccggac catctttgat | 180 |
| atcactaata acctctccat tgtgatcctg gctctgcgcc catctgacga gggcacatac | 240 |
| gagtgtgttg ttctgaagta tgaaaaagac gctttcaagc gggaacacct ggctgaagtg | 300 |
| acg. | 303 |

The immunomodulatory polypeptide can have at least 80%, 85%, 90%, 95%, 99%, or 100% sequence identity to the human amino acid sequence:

| (SEQ ID NO: 44) | |
|---|---|
| VIHVTKEVKE VATLSCGHNV SVEELAQTRI YWQKEKKMVL TMMSGDMNIW PEYKNRTIFD | 60 |
| ITNNLSIVIL ALRPSDEGTY ECVVLKYEKD AFKREHLAEV T, | 101 |
| also referred to as B7.1V. | |

c. B7.1 Extracellular Domain Fragments

Exemplary suitable fragments of murine B7.1 that can be used as a costimulatory polypeptide domain include, but are not limited to, the following:

42-246, 42-245, 42-244, 42-243, 42-242, 42-241, 42-240,

41-246, 41-245, 41-244, 41-243, 41-242, 41-241, 41-240,

40-246, 40-245, 40-244, 40-243, 40-242, 40-241, 40-240,

39-246, 39-245, 39-244, 39-243, 39-242, 39-241, 39-240,

38-246, 38-245, 38-244, 38-243, 38-242, 38-241, 38-240,

37-246, 37-245, 37-244, 37-243, 37-242, 37-241, 37-240,

36-246, 36-245, 36-244, 36-243, 36-242, 36-241, 36-240,

35-246, 35-245, 35-244, 35-243, 35-242, 35-241, 35-240,

34-246, 34-245, 34-244, 34-243, 34-242, 34-241, 34-240,

### of SEQ ID NO:11.

Additional suitable fragments of murine B7.1 include, but are not limited to, the following:

38-246, 38-247, 38-248, 38-249, 38-250, 38-251, 38-252,

39-246, 39-247, 39-248, 39-249, 39-250, 39-251, 39-252,

40-246, 40-247, 40-248, 40-249, 40-250, 40-251, 40-252,

41-246, 41-247, 41-248, 41-249, 41-250, 41-251, 41-252,

42-246, 42-247, 42-248, 42-249, 42-250, 42-251, 42-252,

of SEQ ID NO:11, optionally with one to five amino acids of a signal peptide attached to the N-terminal end. The signal peptide may be any disclosed herein, including the signal peptide contained within SEQ ID NO:11, or may be any signal peptide known in the art.

Exemplary suitable fragments of human B7.1 that can be used as a costimulatory polypeptide domain include, but are not limited to, the following:

39-243, 39-242, 39-241, 39-240, 39-239, 39-238, 39-237,

38-243, 38-242, 38-241, 38-240, 38-239, 38-238, 38-237,

37-243, 37-242, 37-241, 37-240, 37-239, 37-238, 37-237,

36-243, 36-242, 36-241, 36-240, 36-239, 36-238, 36-237,

35-243, 35-242, 35-241, 35-190, 35-239, 35-238, 35-237,

34-243, 34-242, 34-241, 34-240, 34-239, 34-238, 34-237,

33-243, 33-242, 33-241, 33-240, 33-239, 33-238, 33-237,

32-243, 32-242, 32-241, 32-240, 32-239, 32-238, 32-237,

31-243, 31-242, 31-241, 31-240, 31-239, 31-238, 31-237,

### of SEQ ID NO:13.

Additional suitable fragments of human B7.1 include, but are not limited to, the following:

35-243, 35-244, 35-245, 35-246, 35-247, 35-248, 35-249,

36-243, 36-244, 36-245, 36-246, 36-247, 36-248, 36-249,

37-243, 37-244, 37-245, 37-246, 37-247, 37-248, 37-249,

38-243, 38-244, 38-245, 38-246, 38-247, 38-248, 38-249,

39-243, 39-244, 39-245, 39-246, 39-247, 39-248, 39-249,

of SEQ ID NO:13, optionally with one to five amino acids of a signal peptide attached to the N-terminal end. The signal peptide may be any disclosed herein, including the signal peptide contained within SEQ ID NO:13, or may be any signal peptide known in the art.

4. PD-1 Extracellular Domains

a. Human PD-1 Extracellular Domains

In one embodiment, the immunomodulatory polypeptide includes the extracellular domain of human PD-1 or a fragment thereof. The predicted extracellular domain includes a sequence from about amino acid 21 to about amino acid 170 of Swissport Accession No. Q15116. The immunomodulatory polypeptide can have at least 80%, 85%, 90%, 95%, 99%, or 100% sequence identity to the human amino acid sequence:

| (SEQ ID NO: 45) | |
|---|---|
| PGWFLDSPDR PWNPPTFSPA LLVVTEGDNA TFTCSFSNTS ESFVLNWYRM SPSNQTDKLA | 60 |
| AFPEDRSQPG QDCRFRVTQL PNGRDFHMSV VRARRNDSGT YLCGAISLAP KAQIKESLRA | 120 |
| ELRVTERRAE VPTAHPSPSP RPAGQFQTLV. | 150 |

The signal sequence will be removed in the mature protein. Additionally, it will be appreciated that signal peptides from other organisms can be used to enhance the secretion of the protein from a host during manufacture.

In another embodiment, the immunomodulatory polypeptide includes the IgV domain of human PD-1, for example amino acids 35-145.

b. Non-Human Primate PD-1 Extracellular Domains

In one embodiment, the immunomodulatory polypeptide includes the extracellular domain of non-human primate (Cynomolgus) PD-1 or a fragment thereof. Non-human primate (Cynomolgus) PD-1 polypeptides can have at least 80%, 85%, 90%, 95%, 99% or 100% sequence identity to:

| (SEQ ID NO: 16) | |
|---|---|
| 1 | mqipqapwpv vwavlqlgwr pgwflespdr pwnaptfspa lllvtegdna tftcsfsnas |
| 61 | esfvlnwyrm spsnqtdkla afpedrsqpg qdcrfrvtrl pngrdfhmsv vrarrndsgt |
| 121 | ylcgaislap kaqikeslra elrvterrae vptahpspsp rpagqfqalv vgvvggllgs |
| 181 | lvllvwvlav icsraaqgti earrtgqplk edpsavpvfs vdygeldfqw rektpeppap |
| 241 | cypeqteyat ivfpsglgts sparrgsadg prsprplrpe dghcswpl. |

SEQ ID NO:16 contains a signal sequence from amino acids 1 to 20. The signal sequence will be removed in the mature protein. Additionally, signal peptides from other organisms can be used to enhance the secretion of the protein from a host during manufacture.

In another embodiment, the immunomodulatory polypeptide includes the IgV domain of non-human primate PD-1.

c. Murine PD-1 Extracellular Domains

The immunomodulatory polypeptide includes the extracellular domain of murine PD-1 or a fragment thereof. The immunomodulatory polypeptide can have at least 80%, 85%, 90%, 95%, 99%, or 100% sequence identity to the murine amino acid sequence:

(SEQ ID NO: 17)
MWVRQVPWSFTWAVLQLSWQSGWLLEVPNGPWRSLTFYPAWLTVSEGANATFTCSLSNWSEDLMLNWNRL
SPSNQTEKQAAFCNGLSQPVQDARFQIIQLPNRHDFHMNILDTRRNDSGIYLCGAISLHPKAKIEESPGA
ELVVTERILETSTRYPSPSPKPEGRFQGMVIGIMSALVGIPVLLLLAWALAVFCSTSMSEARGAGSKDDT
LKEEPSAAPVPSVAYEELDFQGREKTPELPTACVHTEYATIVFTEGLGASAMGRRGSADGLQGPRPPRHE
DGHCSWPL.

Amino acids 1-20 are a signal sequence which is cleaved to produce the mature protein. Signal peptides from other organisms can be used to enhance the secretion of the protein from a host during manufacture.

d. PD-1 Extracellular Domain Fragments

The PD-1 extracellular domain can contain one or more amino acids from the signal peptide or the putative transmembrane domain of PD-1. During secretion, the number of amino acids of the signal peptide that are cleaved can vary depending on the expression system and the host. Additionally, fragments of PD-1 extracellular domain missing one or more amino acids from the C-terminus or the N-terminus can be used.

Exemplary suitable fragments of murine or human PD-1 that can be used include, but are not limited to, the following:

24-170, 24-169, 24-166, 24-165, 24-164, 24-163, 24-162,

23-170, 23-169, 23-166, 23-165, 23-164, 23-163, 23-162,

22-170, 22-169, 22-166, 22-165, 22-164, 22-163, 22-162,

21-170, 21-169, 21-166, 21-165, 21-164, 21-163, 21-162,

20-170, 20-169, 20-166, 20-165, 20-164, 20-163, 20-162,

19-170, 19-169, 19-166, 19-165, 19-164, 19-163, 19-162,

18-170, 18-169, 18-166, 18-165, 18-164, 18-163, 18-162,

17-170, 17-169, 17-166, 17-165, 17-164, 17-163, 17-162,

16-170, 16-169, 16-166, 16-165, 16-164, 16-163, 16-162,

16-171, 16-172, 16-173, 16-174, 16-175, 16-176, 16-177,

17-171, 17-172, 17-173, 17-174, 17-175, 17-176, 17-177,

18-171, 18-172, 18-173, 18-174, 18-175, 18-176, 18-177,

19-171, 19-172, 19-173, 19-174, 19-175, 19-176, 19-177,

20-171, 20-172, 20-173, 20-174, 20-175, 20-176, 20-177,

21-171, 21-172, 21-173, 21-174, 21-175, 21-176, 21-177,

22-171, 22-172, 22-173, 22-174, 22-175, 22-176, 22-177,

23-171, 23-172, 23-173, 23-174, 23-175, 23-176, 23-177,

24-171, 24-172, 24-173, 24-174, 24-175, 24-176, 24-177,

### of SEQ ID NO:15-17.

E. Variants

1. Variant PD-L2 and PD-L1 Immunomodulatory Agents

Additional immunomodulatory agents include PD-L2 and PD-L1, polypeptides and fragments and fusions thereof that are mutated so that they have increased binding to PD-1 under physiological conditions, or have decreased ability to promote signal transduction through the PD-1 receptor. One embodiment provides isolated PD-L2 and PD-L1 polypeptides that contain one or more amino acid substitutions, deletions, or insertions that inhibit or reduce the ability of the polypeptide to activate PD-1 and transmit an inhibitory signal to a T cell compared to non-mutated PD-L2 or PD-L1. The PD-L2 and PD-L1 polypeptides may be of any species of origin. In one embodiment, the PD-L2 or PD-L1 polypeptide is from a mammalian species. In a preferred embodiment, the PD-L2 or PD-L1 polypeptide is of human or non-human primate origin.

In another embodiment the variant PD-L2 or PD-L1 polypeptide has the same binding activity to PD-1 as wildtype or non-variant PD-L2 or PD-L1 but does not have or has less than 10% ability to stimulate signal transduction through the PD-1 receptor relative to a non-mutated PD-L2 or PD-L1 polypeptide. In other embodiments, the variant PD-L2 or PD-L1 polypeptide has 10%, 20%, 30%, 40%, 50%, 60%, 70%, 80%, 90%, 100% or more binding activity to PD-1 than wildtype PD-L2 or PD-L1 and has less than 50%, 40%, 30%, 20%, or 10% of the ability to stimulate signal transduction through the PD-1 receptor relative to a non-mutated PD-L2 or PD-L1 polypeptide.

A variant PD-L2 or PD-L1 polypeptide can have any combination of amino acid substitutions, deletions or insertions. In one embodiment, isolated PD-L2 or PD-L1 variant polypeptides have a number of amino acid alterations such that their amino acid sequence shares at least 60, 70, 80, 85, 90, 95, 97, 98, 99, 99.5 or 100% identity with an amino acid sequence of a wild type PD-L2 or PD-L1 polypeptide. In a preferred embodiment, PD-L1 variant polypeptides have an amino acid sequence sharing at least 60, 70, 80, 85, 90, 95, 97, 98, 99, 99.5 or 100% identity with the amino acid sequence of a wild type murine, non-human primate or human PD-L2 or PD-L1 polypeptide.

Percent sequence identity can be calculated using computer programs or direct sequence comparison. Preferred computer program methods to determine identity between two sequences include, but are not limited to, the GCG program package, FASTA, BLASTP, and TBLASTN (see, e.g., D. W. Mount, 2001, Bioinformatics: Sequence and Genome Analysis, Cold Spring Harbor Laboratory Press, Cold Spring Harbor, N.Y.). The BLASTP and TBLASTN programs are publicly available from NCBI and other sources. The well-known Smith Waterman algorithm may also be used to determine identity.

Exemplary parameters for amino acid sequence comparison include the following: 1) algorithm from Needleman and Wunsch (J. Mol. Biol., 48:443-453 (1970)); 2) BLOSSUM62 comparison matrix from Hentikoff and Hentikoff (Proc. Natl. Acad. Sci. U.S.A., 89:10915-10919 (1992)) 3) gap penalty=12; and 4) gap length penalty=4. A program useful with these parameters is publicly available as the “gap” program (Genetics Computer Group, Madison, Wis.). The aforementioned parameters are the default parameters for polypeptide comparisons (with no penalty for end gaps).

Alternatively, polypeptide sequence identity can be calculated using the following equation: % identity=(the number of identical residues)/(alignment length in amino acid residues)*100. For this calculation, alignment length includes internal gaps but does not include terminal gaps.

Amino acid substitutions in PD-L2 or PD-L1 polypeptides may be “conservative” or “non-conservative”. As used herein, “conservative” amino acid substitutions are substitutions wherein the substituted amino acid has similar structural or chemical properties, and “non-conservative” amino acid substitutions are those in which the charge, hydrophobicity, or bulk of the substituted amino acid is significantly altered. Non-conservative substitutions will differ more significantly in their effect on maintaining (a) the structure of the peptide backbone in the area of the substitution, for example, as a sheet or helical conformation, (b) the charge or hydrophobicity of the molecule at the target site, or (c) the bulk of the side chain.

Examples of conservative amino acid substitutions include those in which the substitution is within one of the five following groups: 1) small aliphatic, nonpolar or slightly polar residues (Ala, Ser, Thr, Pro, Gly); 2) polar, negatively charged residues and their amides (Asp, Asn, Glu, Gln); polar, positively charged residues (His, Arg, Lys); large aliphatic, nonpolar residues (Met, Leu, Ile, Val, Cys); and large aromatic resides (Phe, Tyr, Trp). Examples of non-conservative amino acid substitutions are those where 1) a hydrophilic residue, e.g., seryl or threonyl, is substituted for (or by) a hydrophobic residue, e.g., leucyl, isoleucyl, phenylalanyl, valyl, or alanyl; 2) a cysteine or proline is substituted for (or by) any other residue; 3) a residue having an electropositive side chain, e.g., lysyl, arginyl, or histidyl, is substituted for (or by) an electronegative residue, e.g., glutamyl or aspartyl; or 4) a residue having a bulky side chain, e.g., phenylalanine, is substituted for (or by) a residue that does not have a side chain, e.g., glycine.

It is understood, however, that substitutions at the recited amino acid positions can be made using any amino acid or amino acid analog. For example, the substitutions at the recited positions can be made with any of the naturally-occurring amino acids (e.g., alanine, aspartic acid, asparagine, arginine, cysteine, glycine, glutamic acid, glutamine, histidine, leucine, valine, isoleucine, lysine, methionine, proline, threonine, serine, phenylalanine, tryptophan, or tyrosine).

Exemplary variant PD-L2 and PD-L1 polypeptides and fragments are provided in Tables 1 and 2 of Example 1 below. These tables indicate amino acid positions that can be mutated to cause increased of decreased binding of these polypeptides to PD-1, as well as the effect of specific amino acid variations on binding to PD-1, as determined by FACS analysis and ELISA. In one embodiment, variant PD-L2 polypeptides contain a substitution at S58 that results in increase binding to PD-1. In one embodiment, the S58 substitution in PD-L2 is serine to tyrosine. In another embodiment, variant PD-L1 polypeptides contain a substitution at E58, A69 and/or C113 that results in increase binding to PD-1. Exemplary substitutions at these positions include, but are not limited to E568S, A69F and C113Y.

While the substitutions described herein are with respect to mouse, non-human primate and human PD-L2 or PD-L1, it is noted that one of ordinary skill in the art could readily make equivalent alterations to conserved amino acids or amino acids in corresponding positions in the homologous polypeptides from other species (e.g., rat, hamster, guinea pig, gerbil, rabbit, dog, cat, horse, pig, sheep or cow). However, since binding has a species-specific component, it is preferable to use human when administering PD-1 antagonists to humans.

In one embodiment, the disclosed isolated variant PD-L2 or PD-L1 polypeptides are antagonists of PD-1 and bind to and block PD-1 without triggering signal transduction through PD-1. By preventing the attenuation of T cells by PD-1 signal transduction, more T cells are available to be activated. Preventing T cell inhibition enhances T cell responses, enhances proliferation of T cells, enhances production and/or secretion of cytokines by T cells, stimulates differentiation and effector functions of T cells or promotes survival of T cells relative to T cells not contacted with a PD-1 antagonist. The T cell response that results from the interaction typically is greater than the response in the absence of the PD-1 antagonist polypeptide. The response of the T cell in the absence of the PD-1 antagonist polypeptide can be no response or can be a response significantly lower than in the presence of the PD-1 antagonist polypeptide. The response of the T cell can be an effector (e.g., CTL or antibody-producing B cell) response, a helper response providing help for one or more effector (e.g., CTL or antibody-producing B cell) responses, or a suppressive response.

Methods for measuring the binding affinity between two molecules are well known in the art. Methods for measuring the binding affinity of variant PD-L2 or PD-L1 polypeptides for PD-1 include, but are not limited to, fluorescence activated cell sorting (FACS), surface plasmon resonance, fluorescence anisotropy, affinity chromatography and affinity selection-mass spectrometry.

The variant polypeptides disclosed herein can be full-length polypeptides, or can be a fragment of a full length polypeptide. Preferred fragments include all or part of the extracellular domain of effective to bind to PD-1. As used herein, a fragment refers to any subset of the polypeptide that is a shorter polypeptide of the full length protein.

2. Variant B7.1 and PD-1 Immunomodulatory Agents

Additional immunomodulatory agents include B7.1 and PD-1 polypeptides and fragments thereof that are modified so that they retain the ability to bind to PD-L2 and/or PD-L1 under physiological conditions, or have increased binding to PD-L2 and/or PD-L1. Such variant PD-1 proteins include the soluble ECD portion of the PD-1 protein that includes mutations, such as the A99L mutation, that increases binding to the natural ligands (Molnar et al., Crystal structure of the complex between programmed death-1 (PD-1) and its ligand PD-L2, PNAS, Vol. 105, pp. 10483-10488 (29 Jul. 2008)). The B7.1 and PD-1 polypeptides may be of any species of origin. In one embodiment, the B7.1 or PD-1 polypeptide is from a mammalian species. In a preferred embodiment, the B7.1 or PD-1 polypeptide is of human or non-human primate origin.

A variant B7.1 or PD-1 polypeptide can have any combination of amino acid substitutions, deletions or insertions. In one embodiment, isolated B7.1 or PD-1 variant polypeptides have an integer number of amino acid alterations such that their amino acid sequence shares at least 60, 70, 80, 85, 90, 95, 97, 98, 99, 99.5 or 100% identity with an amino acid sequence of a wild type B7.1 or PD-1 polypeptide. In a preferred embodiment, B7.1 or PD-1 variant polypeptides have an amino acid sequence sharing at least 60, 70, 80, 85, 90, 95, 97, 98, 99, 99.5 or 100% identity with the amino acid sequence of a wild type murine, non-human primate or human B7.1 or PD-1 polypeptide.

Amino acid substitutions in B7.1 or PD-1 polypeptides may be “conservative” or “non-conservative”. Conservative and non-conservative substitutions are described above.

In one embodiment, the disclosed isolated variant B7.1 or PD-1 polypeptides are antagonists of PD-1 and bind to PD-L2 and/or PD-L1, thereby blocking their binding to endogenous PD-1. By preventing the attenuation of T cells by PD-1 signal transduction, more T cells are available to be activated. Preventing T cell inhibition enhances T cell responses, enhances proliferation of T cells, enhances production and/or secretion of cytokines by T cells, stimulates differentiation and effector functions of T cells or promotes survival of T cells relative to T cells not contacted with a immunomodulatory agent. The T cell response that results from the interaction typically is greater than the response in the absence of the immunomodulatory agent. The response of the T cell in the absence of the immunomodulatory agent can be no response or can be a response significantly lower than in the presence of the immunomodulatory agent. The response of the T cell can be an effector (e.g., CTL or antibody-producing B cell) response, a helper response providing help for one or more effector (e.g., CTL or antibody-producing B cell) responses, or a suppressive response.

The variant polypeptides can be full-length polypeptides, or can be a fragment of a full length polypeptide. Preferred fragments include all or part of the extracellular domain of effective to bind to PD-L2 and/or PD-L1. As used herein, a fragment refers to any subset of the polypeptide that is a shorter polypeptide of the full length protein.

In one embodiment,

F. Fusion Proteins

In some embodiments, the immunomodulatory agents are fusion proteins that contain a first polypeptide domain and a second domain. The fusion protein can either bind to a T cell receptor and/or preferably the fusion protein can bind to and block inhibitory signal transduction into the T cell, for example by competitively binding to PD-1. By interfering with natural inhibitory ligands binding PD-1, the disclosed compositions effectively block signal transduction through PD-1. Suitable polypeptides include variant polypeptides and/or fragments thereof that have increased or decreased binding affinity to inhibitory T cell signal transduction receptors such as PD-1.

The fusion proteins also optionally contain a peptide or polypeptide linker domain that separates the first polypeptide domain from the antigen-binding domain.

Fusion proteins disclosed herein are of formula I:

N—R₁—R₂—R₃—C

wherein “N” represents the N-terminus of the fusion protein, “C” represents the C-terminus of the fusion protein, “R₁” is a PD-L2, PD-L1, B7.1, or PD-1 polypeptide or a antigen-binding targeting domain, “R₂” is an optional peptide/polypeptide linker domain, and “R₃” is a targeting domain or a antigen-binding targeting domain, wherein “R₃” is a polypeptide domain when “R₁” is a antigen-binding targeting domain, and “R₃” is a antigen-binding targeting domain wherein “R₁” is a PD-L2, PD-L1, B7.1, or PD-1 polypeptide, fragment or variant thereof. In a preferred embodiment, “R₁” is a PD-L2, PD-L1, B7.1, or PD-1 polypeptide domain and “R₃” is a antigen-binding targeting domain or a dimerization domain.

Optionally, the fusion proteins additionally contain a domain that functions to dimerize or multimerize two or more fusion proteins. The domain that functions to dimerize or multimerize the fusion proteins can either be a separate domain, or alternatively can be contained within one of one of the other domains (PD-L2, PD-L1, B7.1, or PD-1 polypeptide domain, antigen-binding targeting domain, or peptide/polypeptide linker domain) of the fusion protein.

The fusion proteins can be dimerized or multimerized. Dimerization or multimerization can occur between or among two or more fusion proteins through dimerization or multimerization domains. Alternatively, dimerization or multimerization of fusion proteins can occur by chemical crosslinking The dimers or multimers that are formed can be homodimeric/homomultimeric or heterodimeric/heteromultimeric.

The modular nature of the fusion proteins and their ability to dimerize or multimerize in different combinations provides a wealth of options for targeting molecules that function to enhance an immune response to the tumor cell microenvironment or to immune regulatory tissues.

1. Antigen-Binding Targeting Domain

The fusion proteins also contain antigen-binding targeting domains. In some embodiments, the targeting domains bind to antigens, ligands or receptors that are specific to immune tissue involved in the regulation of T cell activation in response to infectious disease causing agents, cancer, or tumor sites.

Tumor/Tumor-Associated Vasculature Targeting Domains

Antigens, Ligands and Receptors to Target

Tumor-Specific and Tumor-Associated Antigens

In one embodiment the fusion proteins contain a domain that specifically binds to an antigen that is expressed by tumor cells. The antigen expressed by the tumor may be specific to the tumor, or may be expressed at a higher level on the tumor cells as compared to non-tumor cells. Antigenic markers such as serologically defined markers known as tumor associated antigens, which are either uniquely expressed by cancer cells or are present at markedly higher levels (e.g., elevated in a statistically significant manner) in subjects having a malignant condition relative to appropriate controls, are contemplated for use in certain embodiments.

Tumor-associated antigens may include, for example, cellular oncogene-encoded products or aberrantly expressed proto-oncogene-encoded products (e.g., products encoded by the neu, ras, trk, and kit genes), or mutated forms of growth factor receptor or receptor-like cell surface molecules (e.g., surface receptor encoded by the c-erb B gene). Other tumor-associated antigens include molecules that may be directly involved in transformation events, or molecules that may not be directly involved in oncogenic transformation events but are expressed by tumor cells (e.g., carcinoembryonic antigen, CA-125, melonoma associated antigens, etc.) (see, e.g., U.S. Pat. No. 6,699,475; Jager, et al., Int. J. Cancer, 106:817-20 (2003); Kennedy, et al., Int. Rev. Immunol., 22:141-72 (2003); Scanlan, et al. Cancer Immun., 4:1 (2004)).

Genes that encode cellular tumor associated antigens include cellular oncogenes and proto-oncogenes that are aberrantly expressed. In general, cellular oncogenes encode products that are directly relevant to the transformation of the cell, and because of this, these antigens are particularly preferred targets for immunotherapy. An example is the tumorigenic neu gene that encodes a cell surface molecule involved in oncogenic transformation. Other examples include the ras, kit, and trk genes. The products of proto-oncogenes (the normal genes which are mutated to form oncogenes) may be aberrantly expressed (e.g., overexpressed), and this aberrant expression can be related to cellular transformation. Thus, the product encoded by proto-oncogenes can be targeted. Some oncogenes encode growth factor receptor molecules or growth factor receptor-like molecules that are expressed on the tumor cell surface. An example is the cell surface receptor encoded by the c-erbB gene. Other tumor-associated antigens may or may not be directly involved in malignant transformation. These antigens, however, are expressed by certain tumor cells and may therefore provide effective targets. Some examples are carcinoembryonic antigen (CEA), CA 125 (associated with ovarian carcinoma), and melanoma specific antigens.

In ovarian and other carcinomas, for example, tumor associated antigens are detectable in samples of readily obtained biological fluids such as serum or mucosal secretions. One such marker is CA125, a carcinoma associated antigen that is also shed into the bloodstream, where it is detectable in serum (e.g., Bast, et al., N. Eng. J. Med., 309:883 (1983); Lloyd, et al., Int. J. Canc., 71:842 (1997). CA125 levels in serum and other biological fluids have been measured along with levels of other markers, for example, carcinoembryonic antigen (CEA), squamous cell carcinoma antigen (SCC), tissue polypeptide specific antigen (TPS), sialyl TN mucin (STN), and placental alkaline phosphatase (PLAP), in efforts to provide diagnostic and/or prognostic profiles of ovarian and other carcinomas (e.g., Sarandakou, et al., Acta Oncol., 36:755 (1997); Sarandakou, et al., Eur. J. Gynaecol. Oncol., 19:73 (1998); Meier, et al., Anticancer Res., 17(4B):2945 (1997); Kudoh, et al., Gynecol. Obstet. Invest., 47:52 (1999)). Elevated serum CA125 may also accompany neuroblastoma (e.g., Hirokawa, et al., Surg. Today, 28:349 (1998), while elevated CEA and SCC, among others, may accompany colorectal cancer (Gebauer, et al., Anticancer Res., 17(4B):2939 (1997)).

The tumor associated antigen, mesothelin, defined by reactivity with monoclonal antibody K-1, is present on a majority of squamous cell carcinomas including epithelial ovarian, cervical, and esophageal tumors, and on mesotheliomas (Chang, et al., Cancer Res., 52:181 (1992); Chang, et al., Int. J. Cancer, 50:373 (1992); Chang, et al., Int. J. Cancer, 51:548 (1992); Chang, et al., Proc. Natl. Acad. Sci. USA, 93:136 (1996); Chowdhury, et al., Proc. Natl. Acad. Sci. USA, 95:669 (1998)). Using MAb K-1, mesothelin is detectable only as a cell-associated tumor marker and has not been found in soluble form in serum from ovarian cancer patients, or in medium conditioned by OVCAR-3 cells (Chang, et al., Int. J. Cancer, 50:373 (1992)). Structurally related human mesothelin polypeptides, however, also include tumor-associated antigen polypeptides such as the distinct mesothelin related antigen (MRA) polypeptide, which is detectable as a naturally occurring soluble antigen in biological fluids from patients having malignancies (see WO 00/50900).

A tumor antigen may include a cell surface molecule. Tumor antigens of known structure and having a known or described function, include the following cell surface receptors: HER1 (GenBank Accession No. U48722), HER2 (Yoshino, et al., J. Immunol., 152:2393 (1994); Disis, et al., Canc. Res., 54:16 (1994); GenBank Acc. Nos. X03363 and M17730), HER3 (GenBank Acc. Nos. U29339 and M34309), HER4 (Plowman, et al., Nature, 366:473 (1993); GenBank Acc. Nos. L07868 and T64105), epidermal growth factor receptor (EGFR) (GenBank Acc. Nos. U48722, and K03193), vascular endothelial cell growth factor (GenBank No. M32977), vascular endothelial cell growth factor receptor (GenBank Acc. Nos. AF022375, 1680143, U48801 and X62568), insulin-like growth factor-I (GenBank Acc. Nos. X00173, X56774, X56773, X06043, European Patent No. GB 2241703), insulin-like growth factor-II (GenBank Acc. Nos. X03562, X00910, M17863 and M17862), transferrin receptor (Trowbridge and Omary, Proc. Nat. Acad. USA, 78:3039 (1981); GenBank Acc. Nos. X01060 and M11507), estrogen receptor (GenBank Acc. Nos. M38651, X03635, X99101, U47678 and M12674), progesterone receptor (GenBank Acc. Nos. X51730, X69068 and M15716), follicle stimulating hormone receptor (FSH-R) (GenBank Acc. Nos. Z34260 and M65085), retinoic acid receptor (GenBank Acc. Nos. L12060, M60909, X77664, X57280, X07282 and X06538), MUC-1 (Barnes, et al., Proc. Nat. Acad. Sci. USA, 86:7159 (1989); GenBank Acc. Nos. M65132 and M64928) NY-ESO-1 (GenBank Acc. Nos. AJ003149 and U87459), NA 17-A (PCT Publication No. WO 96/40039), Melan-A/MART-1 (Kawakami, et al., Proc. Nat. Acad. Sci. USA, 91:3515 (1994); GenBank Acc. Nos. U06654 and U06452), tyrosinase (Topalian, et al., Proc. Nat. Acad. Sci. USA, 91:9461 (1994); GenBank Acc. No. M26729; Weber, et al., J. Clin. Invest, 102:1258 (1998)), Gp-100 (Kawakami, et al., Proc. Nat. Acad. Sci. USA, 91:3515 (1994); GenBank Acc. No. 573003, Adema, et al., J. Biol. Chem., 269:20126 (1994)), MAGE (van den Bruggen, et al., Science, 254:1643 (1991)); GenBank Acc. Nos. U93163, AF064589, U66083, D32077, D32076, D32075, U10694, U10693, U10691, U10690, U10689, U10688, U10687, U10686, U10685, L18877, U10340, U10339, L18920, UO3735 and M77481), BAGE (GenBank Acc. No. U19180; U.S. Pat. Nos. 5,683,886 and 5,571,711), GAGE (GenBank Acc. Nos. AF055475, AF055474, AF055473, U19147, U19146, U19145, U19144, U19143 and U19142), any of the CTA class of receptors including in particular HOM-MEL-40 antigen encoded by the SSX2 gene (GenBank Acc. Nos. X86175, U90842, U90841 and X86174), carcinoembryonic antigen (CEA, Gold and Freedman, J. Exp. Med., 121:439 (1985); GenBank Acc. Nos. M59710, M59255 and M29540), and PyLT (GenBank Acc. Nos. J02289 and J02038); p97 (melanotransferrin) (Brown, et al., J. Immunol., 127:539-46 (1981); Rose, et al., Proc. Natl. Acad. Sci. USA, 83:1261-61 (1986)).

Additional tumor associated antigens include prostate surface antigen (PSA) (U.S. Pat. Nos. 6,677,157; 6,673,545); β-human chorionic gonadotropin β-HCG) (McManus, et al., Cancer Res., 36:3476-81 (1976); Yoshimura, et al., Cancer, 73:2745-52 (1994); Yamaguchi, et al., Br. J. Cancer, 60:382-84 (1989): Alfthan, et al., Cancer Res., 52:4628-33 (1992)); glycosyltransferase β-1,4-N-acetylgalactosaminyltransferases (GalNAc) (Hoon, et al., Int. J. Cancer, 43:857-62 (1989); Ando, et al., Int. J. Cancer, 40:12-17 (1987); Tsuchida, et al., J. Natl. Cancer, 78:45-54 (1987); Tsuchida, et al., J. Natl. Cancer, 78:55-60 (1987)); NUC18 (Lehmann, et al., Proc. Natl. Acad. Sci. USA, 86:9891-95 (1989); Lehmann, et al., Cancer Res., 47:841-45 (1987)); melanoma antigen gp75 (Vijayasardahi, et al., J. Exp. Med., 171:1375-80 (1990); GenBank Accession No. X51455); human cytokeratin 8; high molecular weight melanoma antigen (Natali, et al., Cancer, 59:55-63 (1987); keratin 19 (Datta, et al., J. Clin. Oncol., 12:475-82 (1994)).

Tumor antigens of interest include antigens regarded in the art as “cancer/testis” (CT) antigens that are immunogenic in subjects having a malignant condition (Scanlan, et al., Cancer Immun., 4:1 (2004)). CT antigens include at least 19 different families of antigens that contain one or more members and that are capable of inducing an immune response, including but not limited to MAGEA (CT1); BAGE (CT2); MAGEB (CT3); GAGE (CT4); SSX (CT5); NY-ESO-1 (CT6); MAGEC(CT7); SYCP1 (C8); SPANXB1 (CT11.2); NA88 (CT18); CTAGE (CT21); SPA17 (CT22); OY-TES-1 (CT23); CAGE (CT26); HOM-TES-85 (CT28); HCA661 (CT30); NY-SAR-35 (CT38); FATE (CT43); and TPTE (CT44).

Additional tumor antigens that can be targeted, including a tumor-associated or tumor-specific antigen, include, but not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pm1-RARα fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, and TPS. Other tumor-associated and tumor-specific antigens are known to those of skill in the art and are suitable for targeting by the disclosed fusion proteins.

Antigens Associated with Tumor Neovasculature

Protein therapeutics can be ineffective in treating tumors because they are inefficient at tumor penetration. Tumor-associated neovasculature provides a readily accessible route through which protein therapeutics can access the tumor. In another embodiment the fusion proteins contain a domain that specifically binds to an antigen that is expressed by neovasculature associated with a tumor.

The antigen may be specific to tumor neovasculature or may be expressed at a higher level in tumor neovasculature when compared to normal vasculature. Exemplary antigens that are over-expressed by tumor-associated neovasculature as compared to normal vasculature include, but are not limited to, VEGF/KDR, Tie2, vascular cell adhesion molecule (VCAM), endoglin and α₅β₃ integrin/vitronectin. Other antigens that are over-expressed by tumor-associated neovasculature as compared to normal vasculature are known to those of skill in the art and are suitable for targeting by the disclosed fusion proteins.

Targeting Domains for Infections

Antigens, Ligands and Receptors to Target

In one embodiment the fusion proteins contain a domain that specifically binds to an antigen that is expressed by immune tissue involved in the regulation of T cell activation in response to infectious disease causing agents.

Ligands and Receptors

In one embodiment, disease targeting domains are ligands that bind to cell surface antigens or receptors that are specifically expressed on diseased cells or are overexpressed on diseased cells as compared to normal tissue. Diseased cells also secrete a large number of ligands into the microenvironment that affect growth and development. Receptors that bind to ligands secreted by diseased cells, including, but not limited to growth factors, cytokines and chemokines, including the chemokines provided above, are suitable for use in the disclosed fusion proteins. Ligands secreted by diseased cells can be targeted using soluble fragments of receptors that bind to the secreted ligands. Soluble receptor fragments are fragments polypeptides that may be shed, secreted or otherwise extracted from the producing cells and include the entire extracellular domain, or fragments thereof.

Single Polypeptide Antibodies

In another embodiment, disease-associated targeting domains are single polypeptide antibodies that bind to cell surface antigens or receptors that are specifically expressed on diseased cells or are overexpressed on diseased cells as compared to normal tissue.

Fc Domains

In another embodiment, disease or disease-associated targeting domains are Fc domains of immunoglobulin heavy chains that bind to Fc receptors expressed on diseased cells. The Fc region a includes the polypeptides containing the constant region of an antibody excluding the first constant region immunoglobulin domain. Thus Fc refers to the last two constant region immunoglobulin domains of IgA, IgD, and IgG, and the last three constant region immunoglobulin domains of IgE and IgM. In a preferred embodiment, the Fc domain is derived from a human or murine immunoglobulin. In a more preferred embodiment, the Fc domain is derived from human IgG1 or murine IgG2a including the C_{H}2 and C_{H}3 regions.

In one embodiment, the hinge, C_{H}2 and C_{H}3 regions of a human immunoglobulin Cγ1 chain are encoded by a nucleic acid having at least 80%, 85%, 90%, 95%, 99% or 100% sequence identity to:

| (SEQ ID NO: 46) | |
|---|---|
| gagcctaagt catgtgacaa gacccatacg tgcccaccct gtcccgctcc agaactgctg | 60 |
| gggggaccta gcgttttctt gttcccccca aagcccaagg acaccctcat gatctcacgg | 120 |
| actcccgaag taacatgcgt agtagtcgac gtgagccacg aggatcctga agtgaagttt | 180 |
| aattggtacg tggacggagt cgaggtgcat aatgccaaaa ctaaacctcg ggaggagcag | 240 |
| tataacagta cctaccgcgt ggtatccgtc ttgacagtgc tccaccagga ctggctgaat | 300 |
| ggtaaggagt ataaatgcaa ggtcagcaac aaagctcttc ccgccccaat tgaaaagact | 360 |
| atcagcaagg ccaagggaca accccgcgag ccccaggttt acacccttcc accttcacga | 420 |
| gacgagctga ccaagaacca ggtgtctctg acttgtctgg tcaaaggttt ctatccttcc | 480 |
| gacatcgcag tggagtggga gtcaaacggg cagcctgaga ataactacaa gaccacaccc | 540 |
| ccagtgcttg atagcgatgg gagctttttc ctctacagta agctgactgt ggacaaatcc | 600 |
| cgctggcagc agggaaacgt tttctcttgt agcgtcatgc atgaggccct ccacaaccat | 660 |
| tatactcaga aaagcctgag tctgagtccc ggcaaa | 696 |

The hinge, C_{H}2 and C_{H}3 regions of a human immunoglobulin Cγ1 chain encoded by SEQ ID NO:44 has the following amino acid sequence:

| (SEQ ID NO: 47) | |
|---|---|
| EPKSCDKTHT CPPCPAPELL GGPSVFLFPP KPKDTLMISR TPEVTCVVVD VSHEDPEVKF | 60 |
| NWYVDGVEVH NAKTKPREEQ YNSTYRVVSV LTVLHQDWLN GKEYKCKVSN KALPAPIEKT | 120 |
| ISKAKGQPRE PQVYTLPPSR DELTKQVSL TCLVKGFYPS DIAVEWESNG QPENNYKTTP | 180 |
| PVLDSDGSFF LYSKLTVDKS RWQQGNVFSC SVMHEALHNH YTQKSLSLSP GK | 232 |

In another embodiment, the Fc domain of a human immunoglobulin Cγ1 chain has at least 80%, 85%, 90%, 95%, 99% or 100% sequence identity to:

| (SEQ ID NO: 48) | |
|---|---|
| ASTKGPSVFP LAPSSKSTSG GTAALGCLVK DYFPEPVTVS WNSGALTSGV HTFPAVLQSS | 60 |
| GLYSLSSVVT VPSSSLGTQT YICNVNHKPS NTKVDKKVEP KSCDKTHTCP PCPAPELLGG | 120 |
| PSVFLFPPKP KDTLMISRTP EVTCVVVDVS HEDPEVKFNW YVDGVEVHNA KTKPREEQYN | 180 |
| STYRVVSVLT VLHQDWLNGK EYKCKVSNKA LPAPIEKTIS KAKGQPREPQ VYTLPPSRDE | 240 |
| LTKNQVSLTC LVKGFYPSDI AVEWESNGQP ENNYKTTPPV LDSDGSFFLY SKLTVDKSRW | 300 |
| QQGNVFSCSV MHEALHNHYT QKSLSLSPGK | 330 |

In another embodiment, the hinge, C_{H}2 and C_{H}3 regions of a murine immunoglobulin Cγ2a chain are encoded by a nucleic acid having at least 80%, 85%, 90%, 95%, 99% or 100% sequence identity to:

| (SEQ ID NO: 49) | |
|---|---|
| gagccaagag gtcctacgat caagccctgc ccgccttgta aatgcccagc tccaaatttg | 60 |
| ctgggtggac cgtcagtctt tatcttcccg ccaaagataa aggacgtctt gatgattagt | 120 |
| ctgagcccca tcgtgacatg cgttgtggtg gatgtttcag aggatgaccc cgacgtgcaa | 180 |
| atcagttggt tcgttaacaa cgtggaggtg cataccgctc aaacccagac ccacagagag | 240 |
| gattataaca gcaccctgcg ggtagtgtcc gccctgccga tccagcatca ggattggatg | 300 |
| agcgggaaag agttcaagtg taaggtaaac aacaaagatc tgccagcgcc gattgaacga | 360 |
| accattagca agccgaaagg gagcgtgcgc gcacctcagg tttacgtcct tcctccacca | 420 |
| gaagaggaga tgacgaaaaa gcaggtgacc ctgacatgca tggtaactga ctttatgcca | 480 |
| gaagatattt acgtggaatg gactaataac ggaaagacag agctcaatta caagaacact | 540 |
| gagcctgttc tggattctga tggcagctac tttatgtact ccaaattgag ggtcgagaag | 600 |
| aagaattggg tcgagagaaa cagttatagt tgctcagtgg tgcatgaggg cctccataat | 660 |
| catcacacca caaagtcctt cagccgaacg cccgggaaa | 699 |

The hinge, C_{H}2 and C_{H}3 regions of a murine immunoglobulin Cγ2a chain encoded by SEQ ID NO:46 has the following amino acid sequence:

| (SEQ ID NO: 50) | |
|---|---|
| EPRGPTIKPC PPCKCPAPNL LGGPSVFIFP PKIKDVLMIS LSPIVTCVVV DVSEDDPDVQ | 60 |
| ISWFVNNVEV HTAQTQTHRE DYNSTLRVVS ALPIQHQDWM SGKEFKCKVN NKDLPAPIER | 120 |
| TISKPKGSVR APQVYVLPPP EEEMTKKQVT LTCMVTDFMP EDIYVEWTNN GKTELNYKNT | 180 |
| EPVLDSDGSY FMYSKLRVEK KNWVERNSYS CSVVHEGLHN HHTTKSFSRT PGK | 233 |

In one embodiment, the Fc domain may contain one or more amino acid insertions, deletions or substitutions that enhance binding to specific Fc receptors that specifically expressed on tumors or tumor-associated neovasculature or are overexpressed on tumors or tumor-associated neovasculature relative to normal tissue. Suitable amino acid substitutions include conservative and non-conservative substitutions, as described above.

The therapeutic outcome in patients treated with rituximab (a chimeric mouse/human IgG1 monoclonal antibody against CD20) for non-Hodgkin's lymphoma or Waldenstrom's macroglobulinemia correlated with the individual's expression of allelic variants of Fcγ receptors with distinct intrinsic affinities for the Fc domain of human IgG1. In particular, patients with high affinity alleles of the low affinity activating Fc receptor CD16A (FcγRIIIA) showed higher response rates and, in the cases of non-Hodgkin's lymphoma, improved progression-free survival. In another embodiment, the Fc domain may contain one or more amino acid insertions, deletions or substitutions that reduce binding to the low affinity inhibitory Fc receptor CD32B (FcγRIIB) and retain wild-type levels of binding to or enhance binding to the low affinity activating Fc receptor CD16A (FcγRIIIA). In a preferred embodiment, the Fc domain contains amino acid insertions, deletions or substitutions that enhance binding to CD16A. A large number of substitutions in the Fc domain of human IgG1 that increase binding to CD16A and reduce binding to CD32B are known in the art and are described in Stavenhagen, et al., Cancer Res., 57(18):8882-90 (2007). Exemplary variants of human IgG1 Fc domains with reduced binding to CD32B and/or increased binding to CD16A contain F243L, R929P, Y300L, V3051 or P296L substitutions. These amino acid substitutions may be present in a human IgG1 Fc domain in any combination. In one embodiment, the human IgG1 Fc domain variant contains a F243L, R929P and Y300L substitution. In another embodiment, the human IgG1 Fc domain variant contains a F243L, R929P, Y300L, V305I and P296L substitution.

Glycophosphatidylinositol Anchor Domain

In another embodiment, disease or disease-associated neovasculature targeting domains are polypeptides that provide a signal for the posttranslational addition of a glycosylphosphatidylinositol (GPI) anchor. GPI anchors are glycolipid structures that are added posttranslationally to the C-terminus of many eukaryotic proteins. This modification anchors the attached protein in the outer leaflet of cell membranes. GPI anchors can be used to attach T cell receptor binding domains to the surface of cells for presentation to T cells. In this embodiment, the GPI anchor domain is C-terminal to the T cell receptor binding domain.

In one embodiment, the GPI anchor domain is a polypeptide that signals for the posttranslational addition addition of a GPI anchor when the polypeptide is expressed in a eukaryotic system. Anchor addition is determined by the GPI anchor signal sequence, which consists of a set of small amino acids at the site of anchor addition (the ω site) followed by a hydrophilic spacer and ending in a hydrophobic stretch (Low, FASEB J., 3:1600-1608 (1989)). Cleavage of this signal sequence occurs in the ER before the addition of an anchor with conserved central components (Low, FASEB J., 3:1600-1608 (1989)) but with variable peripheral moieties (Homans et al., Nature, 333:269-272 (1988)). The C-terminus of a GPI-anchored protein is linked through a phosphoethanolamine bridge to the highly conserved core glycan, mannose(α1-2)mannose(α1-6)mannose(α1-4)glucosamine(α1-6)myo-inositol. A phospholipid tail attaches the GPI anchor to the cell membrane. The glycan core can be variously modified with side chains, such as a phosphoethanolamine group, mannose, galactose, sialic acid, or other sugars. The most common side chain attached to the first mannose residue is another mannose. Complex side chains, such as the N-acetylgalactosamine-containing polysaccharides attached to the third mannose of the glycan core, are found in mammalian anchor structures. The core glucosamine is rarely modified. Depending on the protein and species of origin, the lipid anchor of the phosphoinositol ring is a diacylglycerol, an alkylacylglycerol, or a ceramide. The lipid species vary in length, ranging from 14 to 28 carbons, and can be either saturated or unsaturated. Many GPI anchors also contain an additional fatty acid, such as palmitic acid, on the 2-hydroxyl of the inositol ring. This extra fatty acid renders the GPI anchor resistant to cleavage by PI-PLC.

GPI anchor attachment can be achieved by expression of a fusion protein containing a GPI anchor domain in a eukaryotic system capable of carrying out GPI posttranslational modifications. GPI anchor domains can be used as the tumor or tumor vasculature targeting domain, or can be additionally added to fusion proteins already containing separate tumor or tumor vasculature targeting domains.

In another embodiment, GPI anchor moieties are added directly to isolated T cell receptor binding domains through an in vitro enzymatic or chemical process. In this embodiment, GPI anchors can be added to polypeptides without the requirement for a GPI anchor domain. GPI anchor moieties can be added to fusion proteins described herein having a T cell receptor binding domain and a tumor or tumor vasculature targeting domain. Alternatively, GPI anchors can be added directly to T cell receptor binding domain polypeptides without the requirement for fusion partners encoding tumor or tumor vasculature targeting domains.

2. Peptide or Polypeptide Linker Domain

Fusion proteins optionally contain a peptide or polypeptide linker domain that separates the costimulatory polypeptide domain from the antigen-binding targeting domain.

Hinge Region of Antibodies

In one embodiment, the linker domain contains the hinge region of an immunoglobulin. In a preferred embodiment, the hinge region is derived from a human immunoglobulin. Suitable human immunoglobulins that the hinge can be derived from include IgG, IgD and IgA. In a preferred embodiment, the hinge region is derived from human IgG.

In another embodiment, the linker domain contains a hinge region of an immunoglobulin as described above, and further includes one or more additional immunoglobulin domains. In one embodiment, the additional domain includes the Fc domain of an immunoglobulin. The Fc region as used herein includes the polypeptides containing the constant region of an antibody excluding the first constant region immunoglobulin domain. Thus Fc refers to the last two constant region immunoglobulin domains of IgA, IgD, and IgG, and the last three constant region immunoglobulin domains of IgE and IgM. In a preferred embodiment, the Fc domain is derived from a human immunoglobulin. In a more preferred embodiment, the Fc domain is derived from human IgG including the C_{H}2 and C_{H}3 regions.

In another embodiment, the linker domain contains a hinge region of an immunoglobulin and either the C_{H}1 domain of an immunoglobulin heavy chain or the C_{L} domain of an immunoglobulin light chain. In a preferred embodiment, the C_{H}1 or C_{L} domain is derived from a human immunoglobulin. The C_{L} domain may be derived from either a κ light chain or a λ light chain. In a more preferred embodiment, the C_{H}1 or C_{L} domain is derived from human IgG.

Amino acid sequences of immunoglobulin hinge regions and other domains are well known in the art.

Other Peptide/Polypeptide Linker Domains

Other suitable peptide/polypeptide linker domains include naturally occurring or non-naturally occurring peptides or polypeptides. Peptide linker sequences are at least 2 amino acids in length. Preferably the peptide or polypeptide domains are flexible peptides or polypeptides. A “flexible linker” refers to a peptide or polypeptide containing two or more amino acid residues joined by peptide bond(s) that provides increased rotational freedom for two polypeptides linked thereby than the two linked polypeptides would have in the absence of the flexible linker. Such rotational freedom allows two or more antigen binding sites joined by the flexible linker to each access target antigen(s) more efficiently. Exemplary flexible peptides/polypeptides include, but are not limited to, the amino acid sequences Gly-Ser, Gly-Ser-Gly-Ser (SEQ ID NO:51), Ala-Ser, Gly-Gly-Gly-Ser (SEQ ID NO:52), (Gly₄-Ser)₃ (SEQ ID NO:53), and (Gly₄-Ser)₄ (SEQ ID NO:54). Additional flexible peptide/polypeptide sequences are well known in the art.

3. Dimerization and Multimerization Domains

The fusion proteins optionally contain a dimerization or multimerization domain that functions to dimerize or multimerize two or more fusion proteins. The domain that functions to dimerize or multimerize the fusion proteins can either be a separate domain, or alternatively can be contained within one of the other domains (T cell costimulatory/coinhibitory receptor binding domain, tumor/tumor neovasculature antigen-binding domain, or peptide/polypeptide linker domain) of the fusion protein.

Dimerization Domains

A “dimerization domain” is formed by the association of at least two amino acid residues or of at least two peptides or polypeptides (which may have the same, or different, amino acid sequences). The peptides or polypeptides may interact with each other through covalent and/or non-covalent association(s). Preferred dimerization domains contain at least one cysteine that is capable of forming an intermolecular disulfide bond with a cysteine on the partner fusion protein. The dimerization domain can contain one or more cysteine residues such that disulfide bond(s) can form between the partner fusion proteins. In one embodiment, dimerization domains contain one, two or three to about ten cysteine residues. In a preferred embodiment, the dimerization domain is the hinge region of an immunoglobulin. In this particular embodiment, the dimerization domain is contained within the linker peptide/polypeptide of the fusion protein.

Additional exemplary dimerization domain can be any known in the art and include, but not limited to, coiled coils, acid patches, zinc fingers, calcium hands, a C_{H}1-C_{L} pair, an “interface” with an engineered “knob” and/or “protruberance” as described in U.S. Pat. No. 5,821,333, leucine zippers (e.g., from jun and/or fos) (U.S. Pat. No. 5,932,448), SH2 (src homology 2), SH3 (src Homology 3) (Vidal, et al., Biochemistry, 43, 7336-44 ((2004)), phosphotyrosine binding (PTB) (Zhou, et al., Nature, 378:584-592 (1995)), WW (Sudol, Prog. Biochys. Mol. Bio., 65:113-132 (1996)), PDZ (Kim, et al., Nature, 378: 85-88 (1995); Komau, et al., Science, 269:1737-1740 (1995)) 14-3-3, WD40 (Hu, et al., J Biol. Chem., 273, 33489-33494 (1998)) EH, Lim, an isoleucine zipper, a receptor dimer pair (e.g., interleukin-8 receptor (IL-8R); and integrin heterodimers such as LFA-1 and GPIIIb/IIIa), or the dimerization region(s) thereof, dimeric ligand polypeptides (e.g. nerve growth factor (NGF), neurotrophin-3 (NT-3), interleukin-8 (IL-8), vascular endothelial growth factor (VEGF), VEGF-C, VEGF-D, PDGF members, and brain-derived neurotrophic factor (BDNF) (Arakawa, et al., J. Biol. Chem., 269(45): 27833-27839 (1994) and Radziejewski, et al., Biochem., 32(48): 1350 (1993)) and can also be variants of these domains in which the affinity is altered. The polypeptide pairs can be identified by methods known in the art, including yeast two hybrid screens. Yeast two hybrid screens are described in U.S. Pat. Nos. 5,283,173 and 6,562,576, both of which are herein incorporated by reference in their entireties. Affinities between a pair of interacting domains can be determined using methods known in the art, including as described in Katahira, et al., J. Biol. Chem., 277, 9242-9246 (2002)). Alternatively, a library of peptide sequences can be screened for heterodimerization, for example, using the methods described in WO 01/00814. Useful methods for protein-protein interactions are also described in U.S. Pat. No. 6,790,624.

Multimerization Domains

A “multimerization domain” is a domain that causes three or more peptides or polypeptides to interact with each other through covalent and/or non-covalent association(s). Suitable multimerization domains include, but are not limited to, coiled-coil domains. A coiled-coil is a peptide sequence with a contiguous pattern of mainly hydrophobic residues spaced 3 and 4 residues apart, usually in a sequence of seven amino acids (heptad repeat) or eleven amino acids (undecad repeat), which assembles (folds) to form a multimeric bundle of helices. Coiled-coils with sequences including some irregular distribution of the 3 and 4 residues spacing are also contemplated. Hydrophobic residues are in particular the hydrophobic amino acids Val, Ile, Leu, Met, Tyr, Phe and Trp. Mainly hydrophobic means that at least 50% of the residues must be selected from the mentioned hydrophobic amino acids.

The coiled coil domain may be derived from laminin. In the extracellular space, the heterotrimeric coiled coil protein laminin plays an important role in the formation of basement membranes. Apparently, the multifunctional oligomeric structure is required for laminin function. Coiled coil domains may also be derived from the thrombospondins in which three (TSP-1 and TSP-2) or five (TSP-3, TSP-4 and TSP-5) chains are connected, or from COMP (COMPcc) (Guo, et at., EMBO J., 1998, 17: 5265-5272) which folds into a parallel five-stranded coiled coil (Malashkevich, et al., Science, 274: 761-765 (1996)).

Additional coiled-coil domains derived from other proteins, and other domains that mediate polypeptide multimerization are known in the art and are suitable for use in the disclosed fusion proteins.

4. Exemplary Fusion Proteins

PD-L2

In a preferred embodiment, the immunomodulatory agent is a PD-L2 fusion protein, wherein a fragment of the extracellular domain of PD-L2 is linked to an immunoglobulin Fc domain (B7-DC-Ig). B7-DC-Ig blocks B7-H1 and B7-DC binding to PD-1.

A representative murine PD-L2 fusion protein is encoded by a nucleic acid having at least 80%, 85%, 90%, 95%, 99% or 100% sequence identity to:

| (SEQ ID NO: 55) | |
|---|---|
| atgctgctcc tgctgccgat actgaacctg agcttacaac ttcatcctgt agcagcttta | 60 |
| ttcaccgtga cagcccctaa agaagtgtac accgtagacg tcggcagcag tgtgagcctg | 120 |
| gagtgcgatt ttgaccgcag agaatgcact gaactggaag ggataagagc cagtttgcag | 180 |
| aaggtagaaa atgatacgtc tctgcaaagt gaaagagcca ccctgctgga ggagcagctg | 240 |
| cccctgggaa aggctttgtt ccacatccct agtgtccaag tgagagattc cgggcagtac | 300 |
| cgttgcctgg tcatctgcgg ggccgcctgg gactacaagt acctgacggt gaaagtcaaa | 360 |
| gcttcttaca tgaggataga cactaggatc ctggaggttc caggtacagg ggaggtgcag | 420 |
| cttacctgcc aggctagagg ttatccccta gcagaagtgt cctggcaaaa tgtcagtgtt | 480 |
| cctgccaaca ccagccacat caggaccccc gaaggcctct accaggtcac cagtgttctg | 540 |
| cgcctcaagc ctcagcctag cagaaacttc agctgcatgt tctggaatgc tcacatgaag | 600 |
| gagctgactt cagccatcat tgaccctctg agtcggatgg aacccaaagt ccccagaacg | 660 |
| tgggagccaa gaggtcctac gatcaagccc tgcccgcctt gtaaatgccc agctccaaat | 720 |
| ttgctgggtg gaccgtcagt ctttatcttc ccgccaaaga taaaggacgt cttgatgatt | 780 |
| agtctgagcc ccatcgtgac atgcgttgtg gtggatgttt cagaggatga ccccgacgtg | 840 |
| caaatcagtt ggttcgttaa caacgtggag gtgcataccg ctcaaaccca gacccacaga | 900 |
| gaggattata acagcaccct gcgggtagtg tccgccctgc cgatccagca tcaggattgg | 960 |
| atgagcggga aagagttcaa gtgtaaggta aacaacaaag atctgccagc gccgattgaa | 1020 |
| cgaaccatta gcaagccgaa agggagcgtg cgcgcacctc aggtttacgt ccttcctcca | 1080 |
| ccagaagagg agatgacgaa aaagcaggtg accctgacat gcatggtaac tgactttatg | 1140 |
| ccagaagata tttacgtgga atggactaat aacggaaaga cagagctcaa ttacaagaac | 1200 |
| actgagcctg ttctggattc tgatggcagc tactttatgt actccaaatt gagggtcgag | 1260 |
| aagaagaatt gggtcgagag aaacagttat agttgctcag tggtgcatga gggcctccat | 1320 |
| aatcatcaca ccacaaagtc cttcagccga acgcccggga aatga | 1365 |

The murine PD-L2 fusion protein encoded by SEQ ID NO:55 has the following amino acid sequence:

| (SEQ ID NO: 56) | |
|---|---|
| MLLLLPILNL SLQLHPVAAL FTVTAPKEVY TVDVGSSVSL ECDFDRRECT ELEGIRASLQ | 60 |
| KVENDTSLQS ERATLLEEQL PLGKALFHIP SVQVRDSGQY RCLVICGAAW DYKYLTVKVK | 120 |
| ASYMRIDTRI LEVPGTGEVQ LTCQARGYPL AEVSWQNVSV PANTSHIRTP EGLYQVTSVL | 180 |
| RLKPQPSRNF SCMFWNAHMK ELTSAIIDPL SRMEPKVPRT WEPRGPTIKP CPPCKCPAPN | 240 |
| LLGGPSVFIF PPKIKDVLMI SLSPIVTCVV VDVSEDDPDV QISWFVNNVE VHTAQTQTHR | 300 |
| EDYNSTLRVV SALPIQHQDW MSGKEFKCKV NNKDLPAPIE RTISKPKGSV RAPQVYVLPP | 360 |
| PEEEMTKKQV TLTCMVTDFM PEDIYVEWTN NGKTELNYKN TEPVLDSDGS YFMYSKLRVE | 420 |
| KKNWVERNSY SCSVVHEGLH NHHTTKSFSR TPGK | 454 |

The amino acid sequence of the murine PD-L2 fusion protein of SEQ ID NO:56 without the signal sequence is:

| (SEQ ID NO: 57) | |
|---|---|
| LFTVTAPKEV YTVDVGSSVS LECDFDRREC TELEGIRASL QKVENDTSLQ SERATLLEEQ | 60 |
| LPLGKALFHI PSVQVRDSGQ YRCLVICGAA WDYKYLTVKV KASYMRIDTR ILEVPGTGEV | 120 |
| QLTCQARGYP LAEVSWQNVS VPANTSHIRT PEGLYQVTSV LRLKPQPSRN FSCMFWNAHM | 180 |
| KELTSAIIDP LSRMEPKVPR TWEPRGPTIK PCPPCKCPAP NLLGGPSVFI FPPKIKDVLM | 240 |
| ISLSPIVTCV VVDVSEDDPD VQISWFVNNV EVHTAQTQTH REDYNSTLRV VSALPIQHQD | 300 |
| WMSGKEFKCK VNNKDLPAPI ERTISKPKGS VRAPQVYVLP PPEEEMTKKQ VTLTCMVTDF | 360 |
| MPEDIYVEWT NNGKTELNYK NTEPVLDSDG SYFMYSKLRV EKKNWVERNS YSCSVVHEGL | 420 |
| HNHHTTKSFS RTPGK. | 435 |

A representative human PD-L2 fusion protein is encoded by a nucleic acid having at least 80%, 85%, 90%, 95%, 99% or 100% sequence identity to:

| (SEQ ID NO: 58) | |
|---|---|
| atgatctttc ttctcttgat gctgtctttg gaattgcaac ttcaccaaat cgcggccctc | 60 |
| tttactgtga ccgtgccaaa agaactgtat atcattgagc acgggtccaa tgtgaccctc | 120 |
| gaatgtaact ttgacaccgg cagccacgtt aacctggggg ccatcactgc cagcttgcaa | 180 |
| aaagttgaaa acgacacttc acctcaccgg gagagggcaa ccctcttgga ggagcaactg | 240 |
| ccattgggga aggcctcctt tcatatccct caggtgcagg ttcgggatga gggacagtac | 300 |
| cagtgcatta ttatctacgg cgtggcttgg gattacaagt atctgaccct gaaggtgaaa | 360 |
| gcgtcctatc ggaaaattaa cactcacatt cttaaggtgc cagagacgga cgaggtggaa | 420 |
| ctgacatgcc aagccaccgg ctacccgttg gcagaggtca gctggcccaa cgtgagcgta | 480 |
| cctgctaaca cttctcattc taggacaccc gagggcctct accaggttac atccgtgctc | 540 |
| cgcctcaaac cgcccccagg ccggaatttt agttgcgtgt tttggaatac ccacgtgcga | 600 |
| gagctgactc ttgcatctat tgatctgcag tcccagatgg agccacggac tcatccaact | 660 |
| tgggaaccta aatcttgcga taaaactcat acctgtcccc cttgcccagc ccccgagctt | 720 |
| ctgggaggtc ccagtgtgtt tctgtttccc ccaaaaccta aggacacact tatgatatcc | 780 |
| cgaacgccgg aagtgacatg cgtggttgtg gacgtctcac acgaagaccc ggaggtgaaa | 840 |
| ttcaactggt acgttgacgg agttgaggtt cataacgcta agaccaagcc cagagaggag | 900 |
| caatacaatt ccacctatcg agtggttagt gtactgaccg ttttgcacca agactggctg | 960 |
| aatggaaaag aatacaagtg caaagtatca aacaaggctt tgcctgcacc catcgagaag | 1020 |
| acaatttcta aagccaaagg gcagcccagg gaaccgcagg tgtacacact cccaccatcc | 1080 |
| cgcgacgagc tgacaaagaa tcaagtatcc ctgacctgcc tggtgaaagg cttttaccca | 1140 |
| tctgacattg ccgtggaatg ggaatcaaat ggacaacctg agaacaacta caaaaccact | 1200 |
| ccacctgtgc ttgacagcga cgggtccttt ttcctgtaca gtaagctcac tgtcgataag | 1260 |
| tctcgctggc agcagggcaa cgtcttttca tgtagtgtga tgcacgaagc tctgcacaac | 1320 |
| cattacaccc agaagtctct gtcactgagc ccaggtaaat ga | 1362 |

The human PD-L2 fusion protein encoded by SEQ ID NO:58 has the following amino acid sequence:

| (SEQ ID NO: 59) | |
|---|---|
| MIFLLLMLSL ELQLHQIAAL FTVTVPKELY IIEHGSNVTL ECNFDTGSHV NLGAITASLQ | 60 |
| KVENDTSPHR ERATLLEEQL PLGKASFHIP QVQVRDEGQY QCIIIYGVAW DYKYLTLKVK | 120 |
| ASYRKINTHI LKVPETDEVE LTCQATGYPL AEVSWPNVSV PANTSHSRTP EGLYQVTSVL | 180 |
| RLKPPPGRNF SCVFWNTHVR ELTLASIDLQ SQMEPRTHPT WEPKSCDKTH TCPPCPAPEL | 240 |
| LGGPSVFLFP PKPKDTLMIS RTPEVTCVVV DVSHEDPEVK FNWYVDGVEV HNAKTKPREE | 300 |
| QYNSTYRVVS VLTVLHQDWL NGKEYKCKVS NKALPAPIEK TISKAKGQPR EPQVYTLPPS | 360 |
| RDELTKNQVS LTCLVKGFYP SDIAVEWESN GQPENNYKTT PPVLDSDGSF FLYSKLTVDK | 420 |
| SRWQQGNVFS CSVMHEALHN HYTQKSLSLS PGK | 453 |

The amino acid sequence of the human PD-L2 fusion protein of SEQ ID NO:59 without the signal sequence is:

| (SEQ ID NO: 60) | |
|---|---|
| LFTVTVPKEL YIIEHGSNVT LECNFDTGSH VNLGAITASL QKVENDTSPH RERATLLEEQ | 60 |
| LPLGKASFHI PQVQVRDEGQ YQCIIIYGVA WDYKYLTLKV KASYRKINTH ILKVPETDEV | 120 |
| ELTCQATGYP LAEVSWPNVS VPANTSHSRT PEGLYQVTSV LRLKPPPGRN FSCVFWNTHV | 180 |
| RELTLASIDL QSQMEPRTHP TWEPKSCDKT HTCPPCPAPE LLGGPSVFLF PPKPKDTLMI | 240 |
| SRTPEVTCVV VDVSHEDPEV KFNWYVDGVE VHNAKTKPRE EQYNSTYRVV SVLTVLHQDW | 300 |
| LNGKEYKCKV SNKALPAPIE KTISKAKGQP REPQVYTLPP SRDELTKNQV SLTCLVKGFY | 360 |
| PSDIAVEWES NGQPENNYKT TPPVLDSDGS FFLYSKLTVD KSRWQQGNVF SCSVMHEALH | 420 |
| NHYTQKSLSL SPGK | 434. |

A representative non-human primate (Cynomolgus) PD-L2 fusion protein has the following amino acid sequence:

(SEQ ID NO: 61)
MIFLLLMLSLELQLHQIAALFTVTVPKELYIIEHGSNVTLECNFDTGSHVNLGAITASLQKVENDTSPHRER
ATLLEEQLPLGKASFHIPQVQVRDEGQYQCIIIYGVAWDYKYLTLKVKASYRKINTHILKVPETDEVELTCQ
ATGYPLAEVSWPNVSVPANTSHSRTPEGLYQVTSVLRLKPPPGRNFSCVFWNTHVRELTLASIDLQSQMEPR
THPTWEPKSCDKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVDKSRWQQGNVFSCS
VMHEALHNHYTQKSLSLSPGK

The amino acid sequence of the non-human primate (Cynomolgus) PD-L2 fusion protein of SEQ ID NO:61 without the signal sequence is:

(SEQ ID NO: 62)
LFTVTVPKELYIIEHGSNVTLECNFDTGSHVNLGAITASLQKVENDTSPHRERATLLEEQLPLGKASFHIPQ
VQVRDEGQYQCIIIYGVAWDYKYLTLKVKASYRKINTHILKVPETDEVELTCQATGYPLAEVSWPNVSVPAN
TSHSRTPEGLYQVTSVLRLKPPPGRNFSCVFWNTHVRELTLASIDLQSQMEPRTHPTWEPKSCDKTHTCPPC
PAPELLGGPSVFLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFSCSVMHEALHNHYTQKSLSLSP
GK.

PD-L1

In another embodiment, the immunomodulatory agent is a PD-L1 fusion protein, wherein a fragment of PD-L1 is linked to an immunoglobulin Fc domain (PD-L1-Ig). PD-L1-Ig blocks PD-L1 and PD-L2 binding to PD-1.

A representative human PD-L1 fusion protein is encoded by a nucleic acid having at least 80%, 85%, 90%, 95%, 99% or 100% sequence identity to:

| (SEQ ID NO: 63) | |
|---|---|
| atgaggatat ttgctgtctt tatattcatg acctactggc atttgctgaa cgcatttact | 60 |
| gtcacggttc ccaaggacct atatgtggta gagtatggta gcaatatgac aattgaatgc | 120 |
| aaattcccag tagaaaaaca attagacctg gctgcactaa ttgtctattg ggaaatggag | 180 |
| gataagaaca ttattcaatt tgtgcatgga gaggaagacc tgaaggttca gcatagtagc | 240 |
| tacagacaga gggcccggct gttgaaggac cagctctccc tgggaaatgc tgcacttcag | 300 |
| atcacagatg tgaaattgca ggatgcaggg gtgtaccgct gcatgatcag ctatggtggt | 360 |
| gccgactaca agcgaattac tgtgaaagtc aatgccccat acaacaaaat caaccaaaga | 420 |
| attttggttg tggatccagt cacctctgaa catgaactga catgtcaggc tgagggctac | 480 |
| cccaaggccg aagtcatctg gacaagcagt gaccatcaag tcctgagtgg taagaccacc | 540 |
| accaccaatt ccaagagaga ggagaagctt ttcaatgtga ccagcacact gagaatcaac | 600 |
| acaacaacta atgagatttt ctactgcact tttaggagat tagatcctga ggaaaaccat | 660 |
| acagctgaat tggtcatccc agaactacct ctggcacatc ctccaaatga aagggacaag | 720 |
| acccatacgt gcccaccctg tcccgctcca gaactgctgg ggggacctag cgttttcttg | 780 |
| ttccccccaa agcccaagga caccctcatg atctcacgga ctcccgaagt aacatgcgta | 840 |
| gtagtcgacg tgagccacga ggatcctgaa gtgaagttta attggtacgt ggacggagtc | 900 |
| gaggtgcata atgccaaaac taaacctcgg gaggagcagt ataacagtac ctaccgcgtg | 960 |
| gtatccgtct tgacagtgct ccaccaggac tggctgaatg gtaaggagta taaatgcaag | 1020 |
| gtcagcaaca aagctcttcc cgccccaatt gaaaagacta tcagcaaggc caagggacaa | 1080 |
| ccccgcgagc cccaggttta cacccttcca ccttcacgag acgagctgac caagaaccag | 1140 |
| gtgtctctga cttgtctggt caaaggtttc tatccttccg acatcgcagt ggagtgggag | 1200 |
| tcaaacgggc agcctgagaa taactacaag accacacccc cagtgcttga tagcgatggg | 1260 |
| agctttttcc tctacagtaa gctgactgtg gacaaatccc gctggcagca gggaaacgtt | 1320 |
| ttctcttgta gcgtcatgca tgaggccctc cacaaccatt atactcagaa aagcctgagt | 1380 |
| ctgagtcccg gcaaatga | 1398. |

The human PD-L1 fusion protein encoded by SEQ ID NO:63 has the following amino acid sequence:

| (SEQ ID NO: 64) | |
|---|---|
| MRIFAVFIFM TYWHLLNAFT VTVPKDLYVV EYGSNMTIEC KFPVEKQLDL AALIVYWEME | 60 |
| DKNIIQFVHG EEDLKVQHSS YRQRARLLKD QLSLGNAALQ ITDVKLQDAG VYRCMISYGG | 120 |
| ADYKRITVKV NAPYNKINQR ILVVDPVTSE HELTCQAEGY PKAEVIWTSS DHQVLSGKTT | 180 |
| TTNSKREEKL FNVTSTLRIN TTTNEIFYCT FRRLDPEENH TAELVIPELP LAHPPNERDK | 240 |
| THTCPPCPAP ELLGGPSVFL FPPKPKDTLM ISRTPEVTCV VVDVSHEDPE VKFNWYVDGV | 300 |
| EVHNAKTKPR EEQYNSTYRV VSVLTVLHQD WLNGKEYKCK VSNKALPAPI EKTISKAKGQ | 360 |
| PREPQVYTLP PSRDELTKNQ VSLTCLVKGF YPSDIAVEWE SNGQPENNYK TTPPVLDSDG | 420 |
| SFFLYSKLTV DKSRWQQGNV FSCSVMHEAL HNHYTQKSLS LSPGK | 465 |

The amino acid sequence of the human PD-L1 fusion protein of SEQ ID NO:64 without the signal sequence is:

| (SEQ ID NO: 65) | |
|---|---|
| FTVTVPKDLY VVEYGSNMTI ECKFPVEKQL DLAALIVYWE MEDKNIIQFV HGEEDLKVQH | 60 |
| SSYRQRARLL KDQLSLGNAA LQITDVKLQD AGVYRCMISY GGADYKRITV KVNAPYNKIN | 120 |
| QRILVVDPVT SEHELTCQAE GYPKAEVIWT SSDHQVLSGK TTTTNSKREE KLFNVTSTLR | 180 |
| INTTTNEIFY CTFRRLDPEE NHTAELVIPE LPLAHPPNER THTCPPCPAP ELLGGPSVFL | 240 |
| FPPKPKDTLM ISRTPEVTCV VVDVSHEDPE VKFNWYVDGV EVHNAKTKPR EEQYNSTYRV | 300 |
| VSVLTVLHQD WLNGKEYKCK VSNKALPAPI EKTISKAKGQ PREPQVYTLP PSRDELTKNQ | 360 |
| VSLTCLVKGF YPSDIAVEWE SNGQPENNYK TTPPVLDSDG SFFLYSKLTV DKSRWQQGNV | 420 |
| FSCSVMHEAL HNHYTQKSLS LSPGK | 445. |

A representative murine PD-L1 fusion protein is encoded by a nucleic acid having at least 80%, 85%, 90%, 95%, 99% or 100% sequence identity to:

| (SEQ ID NO: 66) | |
|---|---|
| atgaggatat ttgctggcat tatattcaca gcctgctgtc acttgctacg ggcgtttact | 60 |
| atcacggctc caaaggactt gtacgtggtg gagtatggca gcaacgtcac gatggagtgc | 120 |
| agattccctg tagaacggga gctggacctg cttgcgttag tggtgtactg ggaaaaggaa | 180 |
| gatgagcaag tgattcagtt tgtggcagga gaggaggacc ttaagcctca gcacagcaac | 240 |
| ttcaggggga gagcctcgct gccaaaggac cagcttttga agggaaatgc tgcccttcag | 300 |
| atcacagacg tcaagctgca ggacgcaggc gtttactgct gcataatcag ctacggtggt | 360 |
| gcggactaca agcgaatcac gctgaaagtc aatgccccat accgcaaaat caaccagaga | 420 |
| atttccgtgg atccagccac ttctgagcat gaactaatat gtcaggccga gggttatcca | 480 |
| gaagctgagg taatctggac aaacagtgac caccaacccg tgagtgggaa gagaagtgtc | 540 |
| accacttccc ggacagaggg gatgcttctc aatgtgacca gcagtctgag ggtcaacgcc | 600 |
| acagcgaatg atgttttcta ctgtacgttt tggagatcac agccagggca aaaccacaca | 660 |
| gcggagctga tcatcccaga actgcctgca acacatcctc cacagaacag gactcacgag | 720 |
| ccaagaggtc ctacgatcaa gccctgcccg ccttgtaaat gcccagctcc aaatttgctg | 780 |
| ggtggaccgt cagtctttat cttcccgcca aagataaagg acgtcttgat gattagtctg | 840 |
| agccccatcg tgacatgcgt tgtggtggat gtttcagagg atgaccccga cgtgcaaatc | 900 |
| agttggttcg ttaacaacgt ggaggtgcat accgctcaaa cccagaccca cagagaggat | 960 |
| tataacagca ccctgcgggt agtgtccgcc ctgccgatcc agcatcagga ttggatgagc | 1020 |
| gggaaagagt tcaagtgtaa ggtaaacaac aaagatctgc cagcgccgat tgaacgaacc | 1080 |
| attagcaagc cgaaagggag cgtgcgcgca cctcaggttt acgtccttcc tccaccagaa | 1140 |
| gaggagatga cgaaaaagca ggtgaccctg acatgcatgg taactgactt tatgccagaa | 1200 |
| gatatttacg tggaatggac taataacgga aagacagagc tcaattacaa gaacactgag | 1260 |
| cctgttctgg attctgatgg cagctacttt atgtactcca aattgagggt cgagaagaag | 1320 |
| aattgggtcg agagaaacag ttatagttgc tcagtggtgc atgagggcct ccataatcat | 1380 |
| cacaccacaa agtccttcag ccgaacgccc gggaaatga | 1419. |

The murine PD-L1 fusion protein encoded by SEQ ID NO:66 has the following amino acid sequence:

| (SEQ ID NO: 67) | |
|---|---|
| MRIFAGIIFT ACCHLLRAFT ITAPKDLYVV EYGSNVTMEC RFPVERELDL LALVVYWEKE | 60 |
| DEQVIQFVAG EEDLKPQHSN FRGRASLPKD QLLKGNAALQ ITDVKLQDAG VYCCIISYGG | 120 |
| ADYKRITLKV NAPYRKINQR ISVDPATSEH ELICQAEGYP EAEVIWTNSD HQPVSGKRSV | 180 |
| TTSRTEGMLL NVTSSLRVNA TANDVFYCTF WRSQPGQNHT AELIIPELPA THPPQNRTHE | 240 |
| PRGPTIKPCP PCKCPAPNLL GGPSVFIFPP KIKDVLMISL SPIVTCVVVD VSEDDPDVQI | 300 |
| SWFVNNVEVH TAQTQTHRED YNSTLRVVSA LPIQHQDWMS GKEFKCKVNN KDLPAPIERT | 360 |
| ISKPKGSVRA PQVYVLPPPE EEMTKKQVTL TCMVTDFMPE DIYVEWTNNG KTELNYKNTE | 420 |
| PVLDSDGSYF MYSKLRVEKK NWVERNSYSC SVVHEGLHNH HTTKSFSRTP GK | 472. |

PD-1

In another embodiment, the immunomodulatory agent is a PD-1 fusion protein, wherein a fragment of PD-1 is linked to an immunoglobulin Fc domain (PD-1-Ig). PD-1-Ig blocks PD-L1 and PD-L2 binding to PD-1.

A representative PD-1 fusion protein has the following amino acid sequence:

| (SEQ ID NO: 68) | |
|---|---|
| PGWFLDSPDR PWNPPTFSPA LLVVTEGDNA TFTCSFSNTS ESFVLNWYRM SPSNQTDKLA | 60 |
| AFPEDRSQPG QDCRFRVTQL PNGRDFHMSV VRARRNDSGT YLCGAISLAP KAQIKESLRA | 120 |
| ELRVTERRAE VPTAHPSPSP RPAGQFQTLV THTCPPCPAP ELLGGPSVFL FPPKPKDTLM | 180 |
| ISRTPEVTCV VVDVSHEDPE VKFNWYVDGV EVHNAKTKPR EEQYNSTYRV VSVLTVLHQD | 240 |
| WLNGKEYKCK VSNKALPAPI EKTISKAKGQ PREPQVYTLP PSRDELTKNQ VSLTCLVKGF | 300 |
| YPSDIAVEWE SNGQPENNYK TTPPVLDSDG SFFLYSKLTV DKSRWQQGNV FSCSVMHEAL | 360 |
| HNHYTQKSLS LSPGK | 375. |

A representative non-human primate (Cynomolgus) PD-1 fusion protein is encoded by a nucleic acid having at least 80%, 85%, 90%, 95%, 99% or 100% sequence identity to:

| (SEQ ID NO: 69) | |
|---|---|
| atgcagatcc cgcaagcccc atggcccgtt gtatgggcgg ttcttcaact tggatggaga | 60 |
| ccaggctggt ttctggagag ccccgaccgg ccctggaatg cgccaacgtt cagccctgcc | 120 |
| ctcctcttgg tgaccgaggg tgataacgct accttcacct gctcatttag taacgcctct | 180 |
| gagtcttttg tcctcaattg gtaccggatg agtcccagca accagactga taaactggct | 240 |
| gcatttccgg aggacaggtc ccagcctggg caagactgta ggttccgcgt gaccagactg | 300 |
| cctaacggac gcgacttcca catgagtgtc gtgcgagcca ggcgcaatga ctccggaact | 360 |
| tatctctgcg gtgccatttc cctggcacct aaagctcaga taaaggaatc tttgagagca | 420 |
| gagctgcgcg tgacagaaag gcgggcagaa gtgcccacag ctcatccgtc acctagcccc | 480 |
| agaccagcgg ggcagtttca aatcgaaggc agaatggatc ctaagtcatg tgacaagacc | 540 |
| catacgtgcc caccctgtcc cgctccagaa ctgctggggg gacctagcgt tttcttgttc | 600 |
| cccccaaagc ccaaggacac cctcatgatc tcacggactc ccgaagtaac atgcgtagta | 660 |
| gtcgacgtga gccacgagga tcctgaagtg aagtttaatt ggtacgtgga cggagtcgag | 720 |
| gtgcataatg ccaaaactaa acctcgggag gagcagtata acagtaccta ccgcgtggta | 780 |
| tccgtcttga cagtgctcca ccaggactgg ctgaatggta aggagtataa atgcaaggtc | 840 |
| agcaacaaag ctcttcccgc cccaattgaa aagactatca gcaaggccaa gggacaaccc | 900 |
| cgcgagcccc aggtttacac ccttccacct tcacgagacg agctgaccaa gaaccaggtg | 960 |
| tctctgactt gtctggtcaa aggtttctat ccttccgaca tcgcagtgga gtgggagtca | 1020 |
| aacgggcagc ctgagaataa ctacaagacc acacccccag tgcttgatag cgatgggagc | 1080 |
| tttttcctct acagtaagct gactgtggac aaatcccgct ggcagcaggg aaacgttttc | 1140 |
| tcttgtagcg tcatgcatga ggccctccac aaccattata ctcagaaaag cctgagtctg | 1200 |
| agtcccggca aatga | 1215. |

The non-human primate (Cynomolgus) PD-1 fusion protein encoded by SEQ ID NO:69 has the following amino acid sequence:

| (SEQ ID NO: 70) | |
|---|---|
| MQIPQAPWPV VWAVLQLGWR PGWFLESPDR PWNAPTFSPA LLLVTEGDNA TFTCSFSNAS | 60 |
| ESFVLNWYRM SPSNQTDKLA AFPEDRSQPG QDCRFRVTRL PNGRDFHMSV VRARRNDSGT | 120 |
| YLCGAISLAP KAQIKESLRA ELRVTERRAE VPTAHPSPSP RPAGQFQIEG RMDPKSCDKT | 180 |
| HTCPPCPAPE LLGGPSVFLF PPKPKDTLMI SRTPEVTCVV VDVSHEDPEV KFNWYVDGVE | 240 |
| VHNAKTKPRE EQYNSTYRVV SVLTVLHQDW LNGKEYKCKV SNKALPAPIE KTISKAKGQP | 300 |
| REPQVYTLPP SRDELTKNQV SLTCLVKGFY PSDIAVEWES NGQPENNYKT TPPVLDSDGS | 360 |
| FFLYSKLTVD KSRWQQGNVF SCSVMHEALH NHYTQKSLSL SPGK | 404. |

B7.1

In another embodiment, the immunomodulatory agent is a B7.1 fusion protein, wherein a fragment of B7.1 is linked to an immunoglobulin Fc domain (B7.1-Ig). B7.1 blocks PD-L1 binding to PD-1.

A representative B7.1 fusion protein has the following amino acid sequence:

| (SEQ ID NO: 71) | |
|---|---|
| MGHTRRQGTS PSKCPYLNFF QLLVLAGLSH FCSGVIHVTK EVKEVATLSC GHNVSVEELA | 60 |
| QTRIYWQKEK KMVLTMMSGD MNIWPEYKNR TIFDITNNLS IVILALRPSD EGTYECVVLK | 120 |
| YEKDAFKREH LAEVTLSVKA DFPTPSISDF EIPTSNIRRI ICSTSGGFPE PHLSWLENGE | 180 |
| ELNAINTTVS QDPETELYAV SSKLDFNMTT NHSFMCLIKY GHLRVNQTFN WNTTKQEHFP | 240 |
| DNTHTCPPCP APELLGGPSV FLFPPKPKDT LMISRTPEVT CVVVDVSHED PEVKFNWYVD | 300 |
| GVEVHNAKTK PREEQYNSTY RVVSVLTVLH QDWLNGKEYK CKVSNKALPA PIEKTISKAK | 360 |
| GQPREPQVYT LPPSRDELTK NQVSLTCLVK GFYPSDIAVE WESNGQPENN YKTTPPVLDS | 420 |
| DGSFFLYSKL TVDKSRWQQG NVFSCSVMHE ALHNHYTQKS LSLSPGK | 467. |

5. Bifunctional Proteins

Bifunctional Fusion Proteins

In a preferred embodiment the fusion protein binds to two or more ligands of PD-1. For example, the fusion protein can be engineered to bind PD-1 and a ligand of PD-1, for example PD-L1 or PD-L2. In still another embodiment the fusion protein can be engineered to bind to both PD-L1 and PD-L2.

G. Isolated Nucleic Acid Molecules Encoding PD-1 Receptor Antagonists

Isolated nucleic acid sequences encoding immunomodulatory polypeptides, fragments thereof, variants thereof and fusion proteins thereof are disclosed. As used herein, “isolated nucleic acid” refers to a nucleic acid that is separated from other nucleic acid molecules that are present in a mammalian genome, including nucleic acids that normally flank one or both sides of the nucleic acid in a mammalian genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

Nucleic acids can be in sense or antisense orientation, or can be complementary to a reference sequence encoding a PD-L2, PD-L1, PD-1 or B7.1 polypeptide or variant thereof. Reference sequences include, for example, the nucleotide sequence of human PD-L2, human PD-L1 or murine PD-L2 and murine PD-L1 which are known in the art and discussed above.

Nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Modifications at the base moiety can include deoxyuridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorgan. Med. Chem. 4:5-23. In addition, the deoxyphosphate backbone can be replaced with, for example, a phosphorothioate or phosphorodithioate backbone, a phosphoroamidite, or an alkyl phosphotriester backbone.

### H. Vectors and Host Cells Expressing PD-1 Receptor Antagonists

Nucleic acids, such as those described above, can be inserted into vectors for expression in cells. As used herein, a “vector” is a replicon, such as a plasmid, phage, or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen Life Technologies (Carlsbad, Calif.).

An expression vector can include a tag sequence. Tag sequences, are typically expressed as a fusion with the encoded polypeptide. Such tags can be inserted anywhere within the polypeptide including at either the carboxyl or amino terminus. Examples of useful tags include, but are not limited to, green fluorescent protein (GFP), glutathione S-transferase (GST), polyhistidine, c-myc, hemagglutinin, Flag™ tag (Kodak, New Haven, Conn.), maltose E binding protein and protein A. In one embodiment, the variant PD-L2 fusion protein is present in a vector containing nucleic acids that encode one or more domains of an Ig heavy chain constant region, preferably having an amino acid sequence corresponding to the hinge, C_{H2} and C_{H3} regions of a human immunoglobulin Cγ1 chain.

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell such as a CHO cell) can be used to, for example, produce the immunomodulatory polypeptides described herein.

### I. Antibody Immunomodulatory Agents

Monoclonal and polyclonal antibodies that are reactive with epitopes of the PD-L1, PD-L2, or PD-1, are disclosed. Monoclonal antibodies (mAbs) and methods for their production and use are described in Kohler and Milstein, Nature 256:495-497 (1975); U.S. Pat. No. 4,376,110; Hartlow, E. et al., Antibodies: A Laboratory Manual, Cold Spring Harbor Laboratory Press, Cold Spring Harbor, N.Y., 1988); Monoclonal Antibodies and Hybridomas: A New Dimension in Biological Analyses, Plenum Press, New York, N.Y. (1980); H. Zola et al., in Monoclonal Hybridoma Antibodies: Techniques and Applications, CRC Press, 1982)).

Antibodies that bind to PD-1 and block signal transduction through PD-1, and which have a lower affinity than those currently in use, allowing the antibody to dissociate in a period of less than three months, two months, one month, three weeks, two weeks, one week, or a few days after administration, are preferred for enhancement, augmentation or stimulation of an immune response.

One embodiment includes a bi-specific antibody that comprises an antibody that binds to the PD-L1 ligand bridged to an antibody that binds to the PD-L2 ligand, and prevents both from interacting with PD-1.

Another embodiment includes a bi-specific antibody that comprises an antibody that binds to the PD-1 receptor bridged to an antibody that binds to a ligand of PD-1, such as B7-H1. In a preferred embodiment, the PD-1 binding portion reduces or inhibits signal transduction through the PD-1 receptor. Alternatively, the antibody binds to an epitope that is present on both PD-L1 and PD-L2 and prevents them from interacting with PD-1.

Immunoassay methods are described in Coligan, J. E. et al., eds., Current Protocols in Immunology, Wiley-Interscience, New York 1991 (or current edition); Butt, W. R. (ed.) Practical Immunoassay: The State of the Art, Dekker, N.Y., 1984; Bizollon, Ch. A., ed., Monoclonal Antibodies and New Trends in Immunoassays, Elsevier, N.Y., 1984; Butler, J. E., ELISA (Chapter 29), In: van Oss, C. J. et al., (eds), Immunochemistry, Marcel Dekker, Inc., New York, 1994, pp. 759-803; Butler, J. E. (ed.), Immunochemistry of Solid-Phase Immunoassay, CRC Press, Boca Raton, 1991; Weintraub, B., Principles of Radioimmunoassays, Seventh Training Course on Radioligand Assay Techniques, The Endocrine Society, March, 1986; Work, T. S. et al., Laboratory Techniques and Biochemistry in Molecular Biology, North Holland Publishing Company, NY, (1978) (Chapter by Chard, T., “An Introduction to Radioimmune Assay and Related Techniques”).

Anti-idiotypic antibodies are described, for example, in Idiotypy in Biology and Medicine, Academic Press, New York, 1984; Immunological Reviews Volume 79, 1984; Immunological Reviews Volume 90, 1986; Curr. Top. Microbiol., Immunol. Volume 119, 1985; Bona, C. et al., CRC Crit. Rev. Immunol., pp. 33-81 (1981); Jerme, N K, Ann. Immunol. 125C:373-389 (1974); Jerne, N K, In: Idiotypes—Antigens on the Inside, Westen-Schnurr, I., ed., Editiones Roche, Basel, 1982, Urbain, J. et al., Ann. Immunol. 133D:179-(1982); Rajewsky, K. et al., Ann. Rev. Immunol. 1:569-607 (1983).

The antibodies may be xenogeneic, allogeneic, syngeneic, or modified forms thereof, such as humanized or chimeric antibodies. Antiidiotypic antibodies specific for the idiotype of a specific antibody, for example an anti-PD-L2 antibody, are also included. The term “antibody” is meant to include both intact molecules as well as fragments thereof that include the antigen-binding site and are capable of binding to an epitope. These include, Fab and F(ab′)₂ fragments which lack the Fc fragment of an intact antibody, clear more rapidly from the circulation, and may have less non-specific tissue binding than an intact antibody (Wahl et al., J. Nuc. Med. 24:316-325 (1983)). Also included are Fv fragments (Hochman, J. et al. (1973) Biochemistry 12:1130-1135; Sharon, J. et al. (1976) Biochemistry 15:1591-1594). These various fragments are produced using conventional techniques such as protease cleavage or chemical cleavage (see, e.g., Rousseaux et al., Meth. Enzymol., 121:663-69 (1986)).

Polyclonal antibodies are obtained as sera from immunized animals such as rabbits, goats, rodents, etc. and may be used directly without further treatment or may be subjected to conventional enrichment or purification methods such as ammonium sulfate precipitation, ion exchange chromatography, and affinity chromatography.

The immunogen may include the complete PD-L1, PD-L2, PD-1, or fragments or derivatives thereof. Preferred immunogens include all or a part of the extracellular domain (ECD) of PD-L1, PD-L2 or PD-1, where these residues contain the post-translation modifications, such as glycosylation. Immunogens including the extracellular domain are produced in a variety of ways known in the art, e.g., expression of cloned genes using conventional recombinant methods or isolation from cells of origin.

Monoclonal antibodies may be produced using conventional hybridoma technology, such as the procedures introduced by Kohler and Milstein, Nature, 256:495-97 (1975), and modifications thereof (see above references). An animal, preferably a mouse is primed by immunization with an immunogen as above to elicit the desired antibody response in the primed animal. B lymphocytes from the lymph nodes, spleens or peripheral blood of a primed, animal are fused with myeloma cells, generally in the presence of a fusion promoting agent such as polyethylene glycol (PEG). Any of a number of murine myeloma cell lines are available for such use: the P3-NS1/1-Ag4-1, P3-x63-k0Ag8.653, Sp2/0-Ag14, or HL1-653 myeloma lines (available from the ATCC, Rockville, Md.). Subsequent steps include growth in selective medium so that unfused parental myeloma cells and donor lymphocyte cells eventually die while only the hybridoma cells survive. These are cloned and grown and their supernatants screened for the presence of antibody of the desired specificity, e.g. by immunoassay techniques using PD-L2 or PD-L1 fusion proteins. Positive clones are subcloned, e.g., by limiting dilution, and the monoclonal antibodies are isolated.

Hybridomas produced according to these methods can be propagated in vitro or in vivo (in ascites fluid) using techniques known in the art (see generally Fink et al., Prog. Clin. Pathol., 9:121-33 (1984)). Generally, the individual cell line is propagated in culture and the culture medium containing high concentrations of a single monoclonal antibody can be harvested by decantation, filtration, or centrifugation.

The antibody may be produced as a single chain antibody or scFv instead of the normal multimeric structure. Single chain antibodies include the hypervariable regions from an Ig of interest and recreate the antigen binding site of the native Ig while being a fraction of the size of the intact Ig (Skerra, A. et al. Science, 240: 1038-1041 (1988); Pluckthun, A. et al. Methods Enzymol. 178: 497-515 (1989); Winter, G. et al. Nature, 349: 293-299 (1991)). In a preferred embodiment, the antibody is produced using conventional molecular biology techniques.

### III. Methods of Manufacture

A. Methods for Producing Immunomodulatory Polypeptides and Variants Thereof

Isolated immunomodulatory agents or variants thereof can be obtained by, for example, chemical synthesis or by recombinant production in a host cell. To recombinantly produce an immunomodulatory agent polypeptide, a nucleic acid containing a nucleotide sequence encoding the polypeptide can be used to transform, transduce, or transfect a bacterial or eukaryotic host cell (e.g., an insect, yeast, or mammalian cell). In general, nucleic acid constructs include a regulatory sequence operably linked to a nucleotide sequence encoding an immunomodulatory polypeptide. Regulatory sequences (also referred to herein as expression control sequences) typically do not encode a gene product, but instead affect the expression of the nucleic acid sequences to which they are operably linked.

Useful prokaryotic and eukaryotic systems for expressing and producing polypeptides are well know in the art include, for example, Escherichia coli strains such as BL-21, and cultured mammalian cells such as CHO cells.

In eukaryotic host cells, a number of viral-based expression systems can be utilized to express an immunomodulatory polypeptide. Viral based expression systems are well known in the art and include, but are not limited to, baculoviral, SV40, retroviral, or vaccinia based viral vectors.

Mammalian cell lines that stably express immunomodulatory polypeptides can be produced using expression vectors with appropriate control elements and a selectable marker. For example, the eukaryotic expression vectors pCR3.1 (Invitrogen Life Technologies) and p91023(B) (see Wong et al. (1985) Science 228:810-815) are suitable for expression of variant costimulatory polypeptides in, for example, Chinese hamster ovary (CHO) cells, COS-1 cells, human embryonic kidney 293 cells, NIH3T3 cells, BHK21 cells, MDCK cells, and human vascular endothelial cells (HUVEC). Following introduction of an expression vector by electroporation, lipofection, calcium phosphate, or calcium chloride co-precipitation, DEAE dextran, or other suitable transfection method, stable cell lines can be selected (e.g., by antibiotic resistance to G418, kanamycin, or hygromycin). The transfected cells can be cultured such that the polypeptide of interest is expressed, and the polypeptide can be recovered from, for example, the cell culture supernatant or from lysed cells. Alternatively, a immunomodulatory polypeptide can be produced by (a) ligating amplified sequences into a mammalian expression vector such as pcDNA3 (Invitrogen Life Technologies), and (b) transcribing and translating in vitro using wheat germ extract or rabbit reticulocyte lysate.

Immunomodulatory polypeptides can be isolated using, for example, chromatographic methods such as DEAE ion exchange, gel filtration, and hydroxylapatite chromatography. For example, immunomodulatory polypeptides in a cell culture supernatant or a cytoplasmic extract can be isolated using a protein G column. In some embodiments, variant immunomodulatory polypeptides can be “engineered” to contain an amino acid sequence that allows the polypeptides to be captured onto an affinity matrix. For example, a tag such as c-myc, hemagglutinin, polyhistidine, or Flag™ (Kodak) can be used to aid polypeptide purification. Such tags can be inserted anywhere within the polypeptide, including at either the carboxyl or amino terminus. Other fusions that can be useful include enzymes that aid in the detection of the polypeptide, such as alkaline phosphatase. Immunoaffinity chromatography also can be used to purify costimulatory polypeptides.

Methods for introducing random mutations to produce variant polypeptides are known in the art. Random peptide display libraries can be used to screen for peptides which interact with PD-1, PD-L1 or PD-L2. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially.

B. Methods for Producing Isolated Nucleic Acid Molecules Encoding Immunomodulatory Polypeptides

Isolated nucleic acid molecules encoding immunomodulatory polypeptides can be produced by standard techniques, including, without limitation, common molecular cloning and chemical nucleic acid synthesis techniques. For example, polymerase chain reaction (PCR) techniques can be used to obtain an isolated nucleic acid encoding a variant costimulatory polypeptide. PCR is a technique in which target nucleic acids are enzymatically amplified. Typically, sequence information from the ends of the region of interest or beyond can be employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize a complementary DNA (cDNA) strand. Ligase chain reaction, strand displacement amplification, self-sustained sequence replication or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA 87:1874-1878; and Weiss (1991) Science 254:1292-1293.

Isolated nucleic acids can be chemically synthesized, either as a single nucleic acid molecule or as a series of oligonucleotides (e.g., using phosphoramidite technology for automated DNA synthesis in the 3′ to 5′ direction). For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase can be used to extend the oligonucleotides, resulting in a single, double-stranded nucleic acid molecule per oligonucleotide pair, which then can be ligated into a vector. Isolated nucleic acids can also obtained by mutagenesis. Immunomodulatory polypeptide encoding nucleic acids can be mutated using standard techniques, including oligonucleotide-directed mutagenesis and/or site-directed mutagenesis through PCR. See, Short Protocols in Molecular Biology. Chapter 8, Green Publishing Associates and John Wiley & Sons, edited by Ausubel et al, 1992. Examples of amino acid positions that can be modified include those described herein.

### IV. Formulations

A. Immunomodulatory Agent Formulations

Pharmaceutical compositions including immunomodulatory agents are provided. Pharmaceutical compositions containing peptides or polypeptides may be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration. The compositions may also be administered using bioerodible inserts and may be delivered directly to an appropriate lymphoid tissue (e.g., spleen, lymph node, or mucosal-associated lymphoid tissue) or directly to an organ or tumor. The compositions can be formulated in dosage forms appropriate for each route of administration. Compositions containing antagonists of PD-1 receptors that are not peptides or polypeptides can additionally be formulated for enteral administration.

As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected. Therapeutically effective amounts of immunomodulatory agents cause an immune response to be activated, enhanced, augmented, or sustained, and/or overcome or alleviate T cell exhaustion and/or T cell anergy, and/or activate monocytes, macrophages, dendritic cells and other antigen presenting cells (“APCs”).

In a preferred embodiment, the immunomodulatoryagent is administered in a range of 0.1-20 mg/kg based on extrapolation from tumor modeling and bioavailability. A most preferred range is 5-20 mg of immunomodulatory agent/kg. Generally, for intravenous injection or infusion, dosage may be lower than when administered by an alternative route.

1. Formulations for Parenteral Administration

In a preferred embodiment, the disclosed compositions, including those containing peptides and polypeptides, are administered in an aqueous solution, by parenteral injection. The formulation may also be in the form of a suspension or emulsion. In general, pharmaceutical compositions are provided including effective amounts of a peptide or polypeptide, and optionally include pharmaceutically acceptable diluents, preservatives, solubilizers, emulsifiers, adjuvants and/or carriers. Such compositions include sterile water, buffered saline (e.g., Tris-HCl, acetate, phosphate), pH and ionic strength; and optionally, additives such as detergents and solubilizing agents (e.g., TWEEN® 20, TWEEN 80, Polysorbate 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

2. Controlled Delivery Polymeric Matrices

Compositions containing one or more immunomodulatory polypeptide or nucleic acids encoding the immunomodulatory polypeptide can be administered in controlled release formulations. Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel. The matrix can also be incorporated into or onto a medical device to modulate an immune response, to prevent infection in an immunocompromised patient (such as an elderly person in which a catheter has been inserted or a premature child) or to aid in healing, as in the case of a matrix used to facilitate healing of pressure sores, decubitis ulcers, etc.

Either non-biodegradable or biodegradable matrices can be used for delivery of immunomodulatory polypeptide or nucleic acids encoding them, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl. Polymer Sci., 35:755-774 (1988).

Controlled release oral formulations may be desirable. Antagonists of PD-1 inhibitory signaling can be incorporated into an inert matrix which permits release by either diffusion or leaching mechanisms, e.g., films or gums. Slowly disintegrating matrices may also be incorporated into the formulation. Another form of a controlled release is one in which the drug is enclosed in a semipermeable membrane which allows water to enter and push drug out through a single small opening due to osmotic effects. For oral formulations, the location of release may be the stomach, the small intestine (the duodenum, the jejunem, or the ileum), or the large intestine. Preferably, the release will avoid the deleterious effects of the stomach environment, either by protection of the active agent (or derivative) or by release of the active agent beyond the stomach environment, such as in the intestine. To ensure full gastric resistance an enteric coating (i.e, impermeable to at least pH 5.0) is essential. These coatings may be used as mixed films or as capsules such as those available from Banner Pharmacaps.

The devices can be formulated for local release to treat the area of implantation or injection and typically deliver a dosage that is much less than the dosage for treatment of an entire body. The devices can also be formulated for systemic delivery. These can be implanted or injected subcutaneously.

3. Formulations for Enteral Administration

Antagonists of PD-1 can also be formulated for oral delivery. Oral solid dosage forms are known to those skilled in the art. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the present proteins and derivatives. See, e.g., Remington's Pharmaceutical Sciences, 21st Ed. (2005, Lippincott, Williams & Wilins, Baltimore, Md. 21201) pages 889-964. The compositions may be prepared in liquid form, or may be in dried powder (e.g., lyophilized) form. Liposomal or polymeric encapsulation may be used to formulate the compositions. See also Marshall, K. In: Modern Pharmaceutics Edited by G. S. Banker and C. T. Rhodes Chapter 10, 1979. In general, the formulation will include the active agent and inert ingredients which protect the immunomodulatory agent in the stomach environment, and release of the biologically active material in the intestine.

Liquid dosage forms for oral administration, including pharmaceutically acceptable emulsions, solutions, suspensions, and syrups, may contain other components including inert diluents; adjuvants such as wetting agents, emulsifying and suspending agents; and sweetening, flavoring, and perfuming agents.

B. Vaccines Including Immunomodulatory Agents

Vaccines require strong T cell response to eliminate infected cells. Immunomodulatory agents described herein can be administered as a component of a vaccine to promote, augment, or enhance the primary immune response and effector cell activity and numbers. Vaccines include antigens, the immunomodulatory agent (or a source thereof) and optionally other adjuvants and targeting molecules. Sources of immunomodulatory agent include any of the disclosed PD-L1, PD-L2 or PD-1 polypeptides, fusion proteins, or variants thereof, nucleic acids encoding any of these polypeptides, or host cells containing vectors that express any of these polypeptides.

1. Antigens

Antigens can be peptides, proteins, polysaccharides, saccharides, lipids, nucleic acids, or combinations thereof. The antigen can be derived from a virus, bacterium, parasite, protozoan, fungus, histoplasma, tissue or transformed cell and can be a whole cell or immunogenic component thereof, e.g., cell wall components or molecular components thereof.

Suitable antigens are known in the art and are available from commercial, government and scientific sources. In one embodiment, the antigens are whole inactivated or attenuated organisms. These organisms may be infectious organisms, such as viruses, parasites and bacteria. The antigens may be tumor cells or cells infected with a virus or intracellular pathogen such as gonorrhea or malaria. The antigens may be purified or partially purified polypeptides derived from tumors or viral or bacterial sources. The antigens can be recombinant polypeptides produced by expressing DNA encoding the polypeptide antigen in a heterologous expression system. The antigens can be DNA encoding all or part of an antigenic protein. The DNA may be in the form of vector DNA such as plasmid DNA.

Antigens may be provided as single antigens or may be provided in combination. Antigens may also be provided as complex mixtures of polypeptides or nucleic acids.

i. Viral Antigens

A viral antigen can be isolated from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxyiridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3.

Viral antigens may be derived from a particular strain, or a combination of strains, such as a papilloma virus, a herpes virus, i.e. herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

ii. Bacterial Antigens

Bacterial antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

iii. Parasitic Antigens

Antigens of parasites can be obtained from parasites such as, but not limited to, antigens derived from Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

iv. Tumor Antigens

The antigen can be a tumor antigen, including a tumor-associated or tumor-specific antigen, such as, but not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pm1-RARα fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY—CO-1, RCAS1, SDCCAG16, TA-90 (Mac-2 binding protein\cyclophilin C-associated protein), TAAL6, TAG72, TLP, and TPS. Tumor antigens, such as BCG, may also be used as an immunostimulant to adjuvant.

2. Adjuvants

Optionally, the vaccines may include an adjuvant. The adjuvant can be, but is not limited to, one or more of the following: oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

Adjuvants may also include immunomodulators such as cytokines, interleukins (e.g., IL-1, IL-2, IL-4, IL-5, IL-6, IL-7, IL-12, etc.), interferons

(e.g., interferon-.gamma.), macrophage colony stimulating factor, and tumor necrosis factor. In addition to variant PD-L2 polypeptides, other co-stimulatory molecules, including other polypeptides of the B7 family, may be administered. Such proteinaceous adjuvants may be provided as the full-length polypeptide or an active fragment thereof, or in the form of DNA, such as plasmid DNA.

### IV. Methods of Use

Immunomodulatory agents describe herein can be used to increase IFNγ producing cells and decrease Treg cells at a tumor site or pathogen infected area. Blocking the interaction of ligands with PD-1 produces different results. For example, blocking PD-L1 mediated signal transduction induces robust effector cell responses resulting in increased IFNγ producing cells at a tumor site or site of infection. Blocking PD-L2 mediated signal transduction decreases the number of infiltrating Tregs at a tumor site or site of infection. Thus, the suppressive function of Tregs is reduced at a tumor site or pathogen infected area. A reduction in the number of infiltrating Tregs can lead to an increase in Th17 cell production and/or IL-17 production, and also reduce the number of PD-1 postive cells. Accordingly, a preferred immunomodulatory agent blocks the interaction of both PD-L1 and PD-L2 with PD-1 resulting in increased IFNγ producing cells and decreased Tregs at a tumor site or a pathogen infected area. An exemparly immunmodulatory agent is a B7-DC-Ig fusion protein described above.

Immunomodulatory polypeptide agents and variants thereof, as well as nucleic acids encoding these polypeptides and fusion proteins, or cells expressing immunomodulatory polypeptide can be used to enhance a primary immune response to an antigen as well as increase effector cell function such as increasing antigen-specific proliferation of T cells, enhance cytokine production by T cells, and stimulate differentiation. The immunostimulatory agents can be used to treat cancer.

The immunomodulatory polypeptide agents can be administered to a subject in need thereof in an effective amount to treat one or more symptoms associated with cancer, help overcome T cell exhaustion and/or T cell anergy. Overcoming T cell exhaustion or T cell anergy can be determined by measuring T cell function using known techniques. In certain embodiments, the immunomodulatory polypeptides are engineered to bind to PD-1 without triggering inhibitory signal transduction through PD-1 and retain the ability to costimulate T cells.

In vitro application of the immunomodulatory polypeptide can be useful, for example, in basic scientific studies of immune mechanisms or for production of activated T cells for use in studies of T cell function or, for example, passive immunotherapy. Furthermore, immunomodulatory polypeptide can be added to in vitro assays (e.g., T cell proliferation assays) designed to test for immunity to an antigen of interest in a subject from which the T cells were obtained. Addition of an immunomodulatory polypeptide to such assays would be expected to result in a more potent, and therefore more readily detectable, in vitro response.

A. Administration of Immunomodulatory Agents for Immunoenhancement

1. Treatment of Cancer

The immunomodulatory agents provided herein are generally useful in vivo and ex vivo as immune response-stimulating therapeutics. In general, the disclosed immunomodulatory agent compositions are useful for treating a subject having or being predisposed to any disease or disorder to which the subject's immune system mounts an immune response. The ability of immunomodulatory agents to inhibit or reduce PD-1 signal transaction enables a more robust immune response to be possible. The disclosed compositions are useful to stimulate or enhance immune responses involving T cells.

The disclosed immunomodulatory agents are useful for stimulating or enhancing an immune response in host for treating cancer by administering to a subject an amount of an immunomodulatory agent effective to stimulate T cells in the subject. The types of cancer that may be treated with the provided compositions and methods include, but are not limited to, the following: bladder, brain, breast, cervical, colo-rectal, esophageal, kidney, liver, lung, nasopharangeal, pancreatic, prostate, skin, stomach, uterine, ovarian, testicular and hematologic.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

2. Treatment of Infections

The immunomodulatory agents are generally useful in vivo and ex vivo as immune response-stimulating therapeutics. In a preferred embodiment, the compositions are useful for treating infections in which T cell exhaustion or T cell anergy has occurred causing the infection to remain with the host over a prolonged period of time. Exemplary infections to be treated are chronic infections cause by a hepatitis virus, a human immunodeficiency virus (HIV), a human T-lymphotrophic virus (HTLV), a herpes virus, an Epstein-Barr virus, or a human papilloma virus. It will be appreciated that other infections can also be treated using the immunomodulatory agents. The disclosed compositions are also useful as part of a vaccine. In a preferred embodiment, the type of disease to be treated or prevented is a chronic infectious disease caused by a bacterium, virus, protozoan, helminth, or other microbial pathogen that enters intracellularly and is attacked, i.e., by cytotoxic T lymphocytes.

Chronic infections in human and animal models are associated with a failure of the host immune response to generate and sustain functional CD8+ and CD4+ T-cell populations, which also results in poor antibody responses to neutralize infectivity. This loss of function is referred to as T cell exhaustion. T cell anergy is a tolerance mechanism in which the lymphocyte is intrinsically functionally inactivated following an antigen encounter, but remains alive for an extended period of time in a hyporesponsive state. One method for treating chronic infection is to revitalize exhausted T cells or to reverse T cell exhaustion in a subject as well as overcoming T cell anergy. Reversal of T cell exhaustion can be achieved by interfering with the interaction between PD-1 and its ligands PD-L1 (B7-H1) and PD-L2 (PD-L2). Acute, often lethal, effects of pathogens can be mediated by toxins or other factors that fail to elicit a sufficient immune response prior to the damage caused by the toxin. This may be overcome by interfering with the interaction between PD-1 and its ligands, allowing for a more effective, rapid immune response.

Because viral infections are cleared primarily by T-cells, an increase in T-cell activity is therapeutically useful in situations where more rapid or thorough clearance of an infective viral agent would be beneficial to an animal or human subject. Thus, the immunomodulatory agents can be administered for the treatment of local or systemic viral infections, including, but not limited to, immunodeficiency (e.g., HIV), papilloma (e.g., HPV), herpes (e.g., HSV), encephalitis, influenza (e.g., human influenza virus A), and common cold (e.g., human rhinovirus) viral infections. For example, pharmaceutical formulations including the immunomodulatory agent compositions can be administered topically to treat viral skin diseases such as herpes lesions or shingles, or genital warts. Pharmaceutical formulations of immunomodulatory compositions can also be administered to treat systemic viral diseases, including, but not limited to, AIDS, influenza, the common cold, or encephalitis.

Representative infections that can be treated, include but are not limited to infections cause by microoganisms including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Histoplasma, Hyphomicrobium, Legionella, Leishmania, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, Yersinia, Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Plasmodium vivax, Trypanosoma brucei, Entamoeba histolytica, Toxoplasma gondii, Trichomonas vaginalis and Schistosoma mansoni.

B. Use of Immunomodulatory Agents in Vaccines

The immunomodulatory agents may be administered alone or in combination with any other suitable treatment. In one embodiment the immunomodulatory agent can be administered in conjunction with, or as a component of a vaccine composition as described above. Suitable components of vaccine compositions are described above. The disclosed immunomodulatory agents can be administered prior to, concurrently with, or after the administration of a vaccine. In one embodiment the immunomodulatory agent composition is administered at the same time as administration of a vaccine.

Immunomodulatory agent compositions may be administered in conjunction with prophylactic vaccines, which confer resistance in a subject to subsequent exposure to infectious agents, or in conjunction with therapeutic vaccines, which can be used to initiate or enhance a subject's immune response to a pre-existing antigen, such as a viral antigen in a subject infected with a virus.

The desired outcome of a prophylactic, therapeutic or de-sensitized immune response may vary according to the disease, according to principles well known in the art. For example, an immune response against an infectious agent may completely prevent colonization and replication of an infectious agent, affecting “sterile immunity” and the absence of any disease symptoms. However, a vaccine against infectious agents may be considered effective if it reduces the number, severity or duration of symptoms; if it reduces the number of individuals in a population with symptoms; or reduces the transmission of an infectious agent. Similarly, immune responses against cancer, allergens or infectious agents may completely treat a disease, may alleviate symptoms, or may be one facet in an overall therapeutic intervention against a disease.

The immunomodulatory agents induce an improved effector cell response such as a CD4 T-cell immune response, against at least one of the component antigen(s) or antigenic compositions compared to the effector cell response obtained with the corresponding composition without the immunomodulatory polypeptide. The term “improved effector cell response” refers to a higher effector cell response such as a CD4 T cell response obtained in a human patient after administration of the vaccine composition than that obtained after administration of the same composition without an immunomodulatory polypeptide. For example, a higher CD4 T-cell response is obtained in a human patient upon administration of an immunogenic composition containing an immunomodulatory agent, preferably PD-L2-Ig, and an antigenic preparation compared to the response induced after administration of an immunogenic composition containing the antigenic preparation thereof which is un-adjuvanted. Such a formulation will advantageously be used to induce anti-antigen effector cell response capable of detection of antigen epitopes presented by MHC class II molecules.

The improved effector cell response can be obtained in an immunologically unprimed patient, i.e. a patient who is seronegative to the antigen. This seronegativity may be the result of the patient having never faced the antigen (so-called “naïve” patient) or, alternatively, having failed to respond to the antigen once encountered. Preferably the improved effector cell response is obtained in an immunocompromised subject such as an elderly, typically 65 years of age or above, or an adult younger than 65 years of age with a high risk medical condition (“high risk” adult), or a child under the age of two.

The improved effector cell response can be assessed by measuring the number of cells producing any of the following cytokines: (1) cells producing at least two different cytokines (CD40L, IL-2, IFNγ, TNF-α, IL-17); (2) cells producing at least CD40L and another cytokine (IL-2, TNF-α, IFNγ, IL-17); (3) cells producing at least IL-2 and another cytokine (CD40L, TNF-alpha, IFNγ, IL-17); (4) cells producing at least IFNγ and another cytokine (IL-2, TNF-α, CD40L, IL-17); (5) cells producing at least TNF-α and another cytokine (IL-2, CD40L, IFNγ, IL-17); and (6) cells producing at least IL-17 and another cytokine (TNF-alpha, IL-2, CD40L, IFNγ, IL-17)

An improved effector cell response is present when cells producing any of the above cytokines will be in a higher amount following administration of the vaccine composition compared to the administration of the composition without a immunomodulatory polypeptide. Typically at least one, preferably two of the five conditions mentioned above will be fulfilled. In a preferred embodiment, cells producing all five cytokines (CD40L, IL-2, IFNγ, TNF-α, IL-17) will be present at a higher number in the vaccinated group compared to the un-vaccinated group.

The immunogenic compositions may be administered by any suitable delivery route, such as intradermal, mucosal e.g. intranasal, oral, intramuscular or subcutaneous. Other delivery routes are well known in the art. The intramuscular delivery route is preferred for the immunogenic compositions. Intradermal delivery is another suitable route. Any suitable device may be used for intradermal delivery, for example short needle devices. Intradermal vaccines may also be administered by devices which limit the effective penetration length of a needle into the skin. Jet injection devices which deliver liquid vaccines to the dermis via a liquid jet injector or via a needle which pierces the stratum corneum and produces a jet which reaches the dermis can also be used. Jet injection devices are known in the art. Ballistic powder/particle delivery devices which use compressed gas to accelerate vaccine in powder form through the outer layers of the skin to the dermis can also be used. Additionally, conventional syringes can be used in the classical Mantoux method of intradermal administration.

Another suitable administration route is the subcutaneous route. Any suitable device may be used for subcutaneous delivery, for example classical needle. Preferably, a needle-free jet injector service is used. Needle-free injectors are known in the art. More preferably the device is pre-filled with the liquid vaccine formulation.

Alternatively the vaccine is administered intranasally. Typically, the vaccine is administered locally to the nasopharyngeal area, preferably without being inhaled into the lungs. It is desirable to use an intranasal delivery device which delivers the vaccine formulation to the nasopharyngeal area, without or substantially without it entering the lungs. Preferred devices for intranasal administration of the vaccines are spray devices. Nasal spray devices are commercially available. Nebulizers produce a very fine spray which can be easily inhaled into the lungs and therefore does not efficiently reach the nasal mucosa. Nebulizers are therefore not preferred. Preferred spray devices for intranasal use are devices for which the performance of the device is not dependent upon the pressure applied by the user. These devices are known as pressure threshold devices. Liquid is released from the nozzle only when a threshold pressure is applied. These devices make it easier to achieve a spray with a regular droplet size. Pressure threshold devices suitable for use with the present invention are known in the art and are commercially available.

Preferred intranasal devices produce droplets (measured using water as the liquid) in the range 1 to 200 μm, preferably 10 to 120 μm. Below 10 μm there is a risk of inhalation, therefore it is desirable to have no more than about 5% of droplets below 10 μm. Droplets above 120 μm do not spread as well as smaller droplets, so it is desirable to have no more than about 5% of droplets exceeding 120 μm.

Bi-dose delivery is another feature of an intranasal delivery system for use with the vaccines. Bi-dose devices contain two sub-doses of a single vaccine dose, one sub-dose for administration to each nostril. Generally, the two sub-doses are present in a single chamber and the construction of the device allows the efficient delivery of a single sub-dose at a time. Alternatively, a monodose device may be used for administering the vaccines.

The immunogenic composition may be given in two or more doses, over a time period of a few days, weeks or months. In one embodiment, different routes of administration are utilized, for example, for the first administration may be given intramuscularly, and the boosting composition, optionally containing a immunomodulatory agent, may be administered through a different route, for example intradermal, subcutaneous or intranasal.

The improved effector cell response conferred by the immunogenic composition may be ideally obtained after one single administration. The single dose approach is extremely relevant in a rapidly evolving outbreak situation including bioterrorist attacks and epidemics. In certain circumstances, especially for the elderly population, or in the case of young children (below 9 years of age) who are vaccinated for the first time against a particular antigen, it may be beneficial to administer two doses of the same composition. The second dose of the same composition (still considered as ‘composition for first vaccination’) can be administered during the on-going primary immune response and is adequately spaced in time from the first dose. Typically the second dose of the composition is given a few weeks, or about one month, e.g. 2 weeks, 3 weeks, 4 weeks, 5 weeks, or 6 weeks after the first dose, to help prime the immune system in unresponsive or poorly responsive individuals.

In a specific embodiment, the administration of the immunogenic composition alternatively or additionally induces an improved B-memory cell response in patients administered with the adjuvanted immunogenic composition compared to the B-memory cell response induced in individuals immunized with the un-adjuvanted composition. An improved B-memory cell response is intended to mean an increased frequency of peripheral blood B lymphocytes capable of differentiation into antibody-secreting plasma cells upon antigen encounter as measured by stimulation of in vitro differentiation (see Example sections, e.g. methods of Elispot B cells memory).

In a still another embodiment, the immunogenic composition increases the primary immune response as well as the CD8 T cell response. The administration of a single dose of the immunogenic composition for first vaccination provides better sero-protection and induces an improved CD4 T-cell, or CD8 T-cell immune response against a specific antigen compared to that obtained with the un-adjuvanted formulation. This may result in reducing the overall morbidity and mortality rate and preventing emergency admissions to hospital for pneumonia and other influenza-like illness. This method allows inducing a CD4 T cell response which is more persistent in time, e.g. still present one year after the first vaccination, compared to the response induced with the un-adjuvanted formulation.

Preferably the CD4 T-cell immune response, such as the improved CD4 T-cell immune response obtained in an unprimed subject, involves the induction of a cross-reactive CD4 T helper response. In particular, the amount of cross-reactive CD4 T cells is increased. The term “cross-reactive” CD4 response refers to CD4 T-cell targeting shared epitopes for example between influenza strains.

The dose of immunomodulatory agent enhances an immune response to an antigen in a human. In particular a suitable immunomodulatory agent amount is that which improves the immunological potential of the composition compared to the unadjuvanted composition, or compared to the composition adjuvanted with another immunomodulatory agent amount. Usually an immunogenic composition dose will range from about 0.5 ml to about 1 ml. Typical vaccine doses are 0.5 ml, 0.6 ml, 0.7 ml, 0.8 ml, 0.9 ml or 1 ml. In a preferred embodiment, a final concentration of 50 μg of immunomodulatory agent, preferably PD-L2-Ig, is contained per ml of vaccine composition, or 25 μg per 0.5 ml vaccine dose. In other preferred embodiments, final concentrations of 35.7 μg or 71.4 μg of immunomodulatory agent is contained per ml of vaccine composition. Specifically, a 0.5 ml vaccine dose volume contains 25 μg or 50 μg of immunomodulatory agent per dose. In still another embodiment, the dose is 100 μg or more. Immunogenic compositions usually contain 15 μg of antigen component as measured by single radial immunodiffusion (SRD) (J. M. Wood et al.: J. Biol. Stand. 5 (1977) 237-247; J. M. Wood et al., J. Biol. Stand. 9 (1981) 317-330).

Subjects can be revaccinated with the immunogenic compositions. Typically revaccination is made at least 6 months after the first vaccination(s), preferably 8 to 14 months after, more preferably at around 10 to 12 months after.

The immunogenic composition for revaccination (the boosting composition) may contain any type of antigen preparation, either inactivated or live attenuated. It may contain the same type of antigen preparation, for example split influenza virus or split influenza virus antigenic preparation thereof, a whole virion, a purified subunit vaccine or a virosome, as the immunogenic composition used for the first vaccination. Alternatively the boosting composition may contain another type of antigen, i.e. split influenza virus or split influenza virus antigenic preparation thereof, a whole virion, a purified subunit vaccine or a virosome, than that used for the first vaccination.

With regard to vaccines against a virus, a boosting composition, where used, is typically given at the next viral season, e.g. approximately one year after the first immunogenic composition. The boosting composition may also be given every subsequent year (third, fourth, fifth vaccination and so forth). The boosting composition may be the same as the composition used for the first vaccination.

Preferably revaccination induces any, preferably two or all, of the following: (i) an improved effector cell response against the antigenic preparation, or (ii) an improved B cell memory response or (iii) an improved humoral response, compared to the equivalent response induced after a first vaccination with the antigenic preparation without a Immunomodulatory agent. Preferably the immunological responses induced after revaccination with the immunogenic antigenic preparation containing the Immunomodulatory agent are higher than the corresponding response induced after the revaccination with the un-adjuvanted composition.

The immunogenic compositions can be monovalent or multivalent, i.e, bivalent, trivalent, or quadrivalent. Preferably the immunogenic composition thereof is trivalent or quadrivalent. Multivalent refers to the number of sources of antigen, typically from different species or strains. With regard to viruses, at least one strain is associated with a pandemic outbreak or has the potential to be associated with a pandemic outbreak.

C. Targeting Antigen Presenting Cells

Another embodiment provides contacting antigen presenting cells (APCs) with one or more of the disclosed immunomodulatory agents in an amount effective to inhibit, reduce or block PD-1 signal transduction in the APCs. Blocking PD-1 signal transduction in the APCs reinvigorates the APCs enhancing clearance of intracellular pathogens, or cells infected with intracellular pathogens.

D. Combination Therapies

The immunomodulatory agent compositions can be administered to a subject in need thereof alone or in combination with one or more additional therapeutic agents. The additional therapeutic agents are selected based on the condition, disorder or disease to be treated. For example, an immunomodulatory agent can be co-administered with one or more additional agents that function to enhance or promote an immune response.

In a preferred embodiment, the additional therapeutic agent is cyclophosphamide. Cyclophosphamide (CPA, Cytoxan, or Neosar) is an oxazahosphorine drug and analogs include ifosfamide (IFO, Ifex), perfosfamide, trophosphamide (trofosfamide; Ixoten), and pharmaceutically acceptable salts, solvates, prodrugs and metabolites thereof (US patent application 20070202077 which is incorporated in its entirety). Ifosfamide (MITOXANAO) is a structural analog of cyclophosphamide and its mechanism of action is considered to be identical or substantially similar to that of cyclophosphamide. Perfosfamide (4-hydroperoxycyclophosphamide) and trophosphamide are also alkylating agents, which are structurally related to cyclophosphamide. For example, perfosfamide alkylates DNA, thereby inhibiting DNA replication and RNA and protein synthesis. New oxazaphosphorines derivatives have been designed and evaluated with an attempt to improve the selectivity and response with reduced host toxicity (Ref. Liang J, Huang M, Duan W, Yu X Q, Zhou S. Design of new oxazaphosphorine anticancer drugs. Curr Pharm Des. 2007; 13(9):963-78. Review). These include mafosfamide (NSC 345842), glufosfamide (D19575, beta-D-glucosylisophosphoramide mustard), S-(−)-bromofosfamide (CBM-11), NSC 612567 (aldophosphamide perhydrothiazine) and NSC 613060 (aldophosphamide thiazolidine). Mafosfamide is an oxazaphosphorine analog that is a chemically stable 4-thioethane sulfonic acid salt of 4-hydroxy-CPA. Glufosfamide is IFO derivative in which the isophosphoramide mustard, the alkylating metabolite of IFO, is glycosidically linked to a beta-D-glucose molecule. Additional cyclophosphamide analogs are described in U.S. Pat. No. 5,190,929 entitled “Cyclophosphamide analogs useful as anti-tumor agents” which is incorporated herein by reference in its entirety.

Additional therapeutic agents include is an agent that reduces activity and/or number of regulatory T lymphocytes (T-regs), preferably Sunitinib (SUTENT®), anti-TGFβ or Imatinib (GLEEVAC®). The recited treatment regimen may also include administering an adjuvant. Other additional therapeutic agents include mitosis inhibitors, such as paclitaxol, aromatase inhibitors (e.g. Letrozole), agniogenesis inhibitors (VEGF inhibitors e.g. Avastin, VEGF-Trap), anthracyclines, oxaliplatin, doxorubicin, TLR4 antagonists, and IL-18 antagonists.

E. Modulating Binding Properties

Binding properties of the immunomodulatory agent are relevant to the dose and dose regime to be administered. Existing antibody Immunomodulatory agents such as MDX-1106 demonstrate sustained occupancy of 60-80% of PD-1 molecules on T cells for at least 3 months following a single dose (Brahmer, et al. J. Clin. Oncology, 27:(155) 3018 (2009)). In preferred embodiments, the disclosed immunomodulatory agents have binding properties to PD-L1/PD-L2/PD-1 that demonstrate a shorter term, or lower percentage, of occupancy of PD-L1/PD-L2/PD-1 molecules on immune cells. For example, the disclosed immunomodulatory agents typically show less than 5, 10, 15, 20, 25, 30, 35, 40, 45, of 50% occupancy of PD-1 molecules on immune cells after one week, two weeks, three weeks, or even one month after administration of a single dose. In other embodiments, the disclosed immunomodulatory agents have reduced binding affinity to PD-1 relative to MDX-1106. In relation to an antibody such as MDX-1106, the PD-L2-Ig fusion protein has a relatively modest affinity for its receptor, and should therefore have a relatively fast off rate.

In other embodiments, the immunomodulatory agents are administered intermittently over a period of days, weeks or months to elicit periodic enhanced immune response which are allowed to diminish prior to the next administration, which may serve to initiate an immune response, stimulate an immune response, or enhance an immune response. In another aspect, methods are provided for modulating an immune response comprising administering to a mammal a composition comprising at least one immunomodulatory agent wherein said immunomodulatory agent provides a maximum plasma concentration of at least about 10 ng/mL. In some aspects, the immunomodulating agent is AMP-224. AMP-224 can be administered as a bolus dose at a dosage of, for example, 1.5 mg/kg, 5 mg/kg, 10 mg/kg, 30 mg/kg and/or 45 mg/kg. In another aspect, AMP-224 has an AUC value that is about 18,000 μg/mL to about 25,000 μg/mL×day over the period of about a week. In yet another aspect, the half-life of the immunomodulatory agent is about 5 to 10 days.

The current invention also provides use of at least one immunomodulatory agent in the manufacture of a medicament for the treatment of diseases, wherein said at least one immunomodulatory agent is formulated for administration to provide a maximum plasma concentration of said at least one immunomodulatory agent of least about 10 ng/mL and an Area Under the Curve value of said at least one immunomodulatory agent which is at least about 18,000 μg/mL to about 25,000 μg/mL×day over the period of one week. In one aspect the present invention provides the use of AMP-224 formulated for administration to provide a maximum plasma concentration of at least about 10 ng/mL.

### EXAMPLES

The present invention may be further understood by reference to the following non-limiting examples.

### Example 1

### Mutagenesis Analysis of PD-1 Receptor Binding Sites of B7-DC and B7-H1

Materials and Methods:

Mice and Cell Lines:

Female C57BL/6 (B6) mice were purchased from the National Cancer Institute (Frederick, Md.). PD-1-deficient (PD-1^{-/-}) mice were generated as described previously (Nishimura, et al., Int. Immunol., 10:1563-1572 (1998)). Stably transfected Chinese hamster ovary (CHO) cell clones secreting fusion proteins were maintained in CHO—SF II medium (Invitrogen Life Technologies) supplemented with 1% dialyzed fetal bovine serum (FBS; HyClone, Logan, Utah). Lymphocytes and COS cells were grown in Dulbecco's modified Eagle medium (DMEM; Invitrogen Life Technologies) supplemented with 10% FBS, 25 mM HEPES, 2 mM L-glutamine, 1 mM sodium pyruvate, 1% MEM nonessential amino acids, 100 U/ml penicillin G, and 100 μg/ml streptomycin sulfate.

Site-Directed Mutagenesis:

All variants of B7-DC-Ig and B7-H1-Ig were constructed using a two-step PCR technique using B7-DC-Ig cDNA as a template. Overlapping oligonucleotide primers were synthesized to encode the desired mutations, and two flanking 5′ and 3′ primers were designed to contain EcoR I and Bgl II restriction sites, respectively. Appropriate regions of the cDNAs initially were amplified using the corresponding overlapping and flanking primers. Using the flanking 5′ and 3′ primers, fragments with overlapping sequences were fused together and amplified. PCR products were digested with EcoR I and Bgl II and ligated into EcoR I/Bgl II-digested pHIg vectors. To verify that the desired mutations were introduced, each variant was sequenced using an ABI Prism 310 Genetic Analyzer. Plasmids were transfected into COS cells, and serum-free supernatants were harvested and used for in vitro binding assays or isolated on a protein G column for BIAcore analysis and functional assays.

Ig Fusion Proteins:

Fusion proteins containing the extracellular domain of mouse PD-1 linked to the Fc portion of mouse IgG2a (PD-1-Ig) were produced in stably transfected CHO cells and isolated by protein G affinity column as described previously (Wand, et al. supra). Total RNA was isolated from mouse spleen cells and B7-DC cDNA was obtained by reverse-transcription PCR. Murine B7-DC-Ig and B7-H1-Ig were prepared by transiently transfecting COS cells with a plasmid containing a chimeric cDNA that included the extracellular domain of mouse B7-DC linked in frame to the CH2-CH3 portion of human IgG1. Human B7-DC-Ig and B7-H1-Ig were prepared by transiently transfecting COS cells with a plasmid containing a chimeric cDNA that included the extracellular domain of human B7-DC linked in frame to the CH2-CH3 portion of human IgG1. The transfected COS cells were cultured in serum-free DMEM, and concentrated supernatants were used as sources of Ig fusion proteins for initial binding assays. The Ig proteins were further isolated on a protein G column for BIAcore analysis and functional assays as described previously (Wand, et al. supra).

Molecular Modeling:

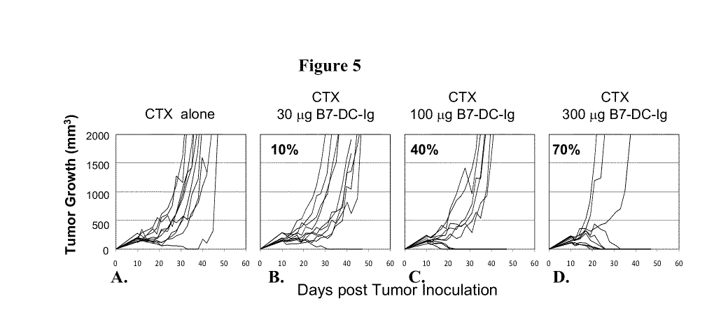

Molecular models of the Ig V-type domains of human B7-H1 (hB7-H1), mouse B7-H1 (mB7-H1), human B7-DC (hB7-DC), and mouse B7-DC (mB7-DC) were generated by homology (or comparative) modeling based on X-ray coordinates of human CD80 and CD86, as seen in the structures of the CD80/CTLA-4 and CD86/CTLA-4 complexes. First, the V-domains of CD80 and CD86 were optimally superimposed, and sequences of B7 family members were aligned based on this superimposition. The superimposition and initial alignments were carried out using the sequence-structure alignment function of MOE (Molecular Operating Environment, Chemical Computing Group, Montreal, Quebec, Canada). The alignment was then manually adjusted to match Ig consensus positions and to map other conserved hydrophobic residues in the target sequences to core positions in the X-ray structures. Corresponding residues in the aligned sequences thus were predicted to have roughly equivalent spatial positions. Taking this kind of structural information into account typically is a more reliable alignment criterion than sequence identity alone if the identity is low, as in this case. In the aligned region, the average identity of the compared B7 sequences relative to the two structural templates, CD80 and CD86, was only approximately 16%. The final version of the structure-oriented sequence alignment, which provided the basis for model building, is shown in FIG. 5. Following the alignment, core regions of the four models were automatically assembled with MOE from the structural templates, and insertions and deletions in loop regions were modeled by applying a segment matching procedure (Levitt, J. Mol. Biol., 226:507-533 (1992); and Fechteler, et al., J. Mol. Biol., 253:114-131 (1995)). Side chain replacements were carried out using preferred rotamer conformations seen in high-resolution protein databank structures (Ponder and Richards, J. Mol. Biol., 193:775-791 (1987); and Berman, et al., Nucl. Acids Res., 28:235-242 (2000)). In each case, twenty intermediate models were generated, average coordinates were calculated, and the resulting structures were energy minimized using a protein force field (Engh and Huber, Ada Cryst., A47:392-400 (1991)) until intramolecular contacts and stereochemistry of each model were reasonable. Graphical analysis of the models, including calculation of solvent-accessible surfaces (Connolly, J. Appl. Cryst., 16:548-558 (1983)) and residue mapping studies were carried out with Insightll (Accelrys, San Diego, Calif.).

EL1SA:

A sandwich ELISA specific for B7-DC-Ig and B7-H1-Ig was established. Microtiter plates were coated with 2 fig/ml goat anti-human IgG (Sigma, St. Louis, Mo.) overnight at 4° C. Wells were blocked for 1 hour with blocking buffer (10% FBS in PBS) and washed with PBS containing 0.05% Tween 20 (PBS-Tween). COS cell culture supernatants were added and incubated for 2 hours at room temperature. Known concentrations of isolated B7-DC-Ig also were added to separate wells on each plate for generation of a standard curve. After extensive washing, horseradish peroxidase (HRP)-conjugated goat anti-human IgG (TAGO, Inc., Burlingame, Calif.) diluted 1:2000 was added and subsequently developed with TMB substrate before stopping the reaction by the addition of 0.5 M H₂SO₄. Absorbance was measured at 405 mm on a microtiter plate reader. Concentrations of variant fusion proteins were determined by comparison with the linear range of a standard curve of B7-DC-Ig and B7-H1-Ig. Data from triplicate wells were collected, and the standard deviations from the mean were <10%. Experiments were repeated at least three times.

The ability of mutant and wild type B7-DC-Ig and B7-H1-Ig fusion polypeptides to bind PD-1 was measured using a capture ELISA assay. Recombinant PD-1Ig fusion proteins were coated on microtiter plates at 5 μg/ml overnight at 4° C. The plates were blocked and washed, and COS cell culture media was added and incubated for 2 hours at room temperature. After extensive washing, HRP-conjugated goat anti-human IgG was added, followed by TMB substrate and measurement of absorbance at 405 mm.

Flow Cytometry:

Human embryonal kidney 293 cells were transfected with a PD-1 GFP vector, which was constructed by fusing GFP (green fluorescent protein cDNA) in frame to the C terminal end of a full-length mouse PD-1 cDNA. The cells were harvested 24 hours after transfection and incubated in FACS (fluorescence activated cell sorting) buffer (PBS, 3% FBS, 0.02% NaN₃) with equal amounts of fusion proteins, which had been titrated using wild type B7-DC-Ig and B7-H1-Ig in COS cell culture media on ice for 45 minutes. An unrelated fusion protein containing human Ig was used as a negative control. The cells were washed, further incubated with fluorescein isothiocyanate (PE)-conjugated goat anti-human IgG (BioSource, Camarillo, Calif.), and analyzed on a FACScaliber (Becton Dickinson, Mountain View, Calif.) with Cell Quest software (Becton Dickinson). GFP-positive cells were gated by FL1.

Surface Plasmon Resonance Analysis:

The affinity of isolated wild type and variant B7-DC polypeptides was analyzed on a BIAcore™ 3000 instrument (Biacore AB, Uppsala, Sweden). All reagents except fusion proteins were purchased, pre-filtered, and degassed from BIAcore. All experiments were performed at 25° C. using 0.1 M HEPES, 0.15 M NaCl (pH 7.4) as a running buffer. Briefly, PD-1Ig was first immobilized onto a CM5 sensor chip (BIAcore) by amine coupling according to the BIAcore protocol. A flow cell of the CM5 chip was derivatized through injection of a 1:1 EDC:NHS [N-ethyl-N′-(diethylaminopropyl) carbodiimide:N-hydroxysuccinimide] mixture for seven minutes, followed by injection of 20 μg/ml of PD-1-Ig at 10 μl/min diluted in 10 mM sodium acetate (pH 4.5). The PD-1-Ig was immobilized at 2000 RUs. This was followed by blocking the remaining activated carboxyl groups with 1 M ethanolamine (pH 8.5). A control flow cell was prepared in a similar fashion as above, substituting running buffer alone in place of PD-1-Ig. The fusion proteins were diluted in running buffer in a concentration series of 3.75, 7.5, 15, 30, and 60 μg/ml. The proteins were injected at a flow rate of 20 μl/min for 3 minutes, and buffer was allowed to flow over the surface for 5 minutes for dissociation data. The flow cells were regenerated with a single 30-second pulse of 10 mM NaOH. Data analysis was performed using BlAevaluation software package 3.1 (BIAcore).

Results:

With the aid of the molecular models, the V-domains of B7-DC and B7-H1 were scanned for important residues, as disclosed in Wang, et al., J. Exp. Med., 197(9):1083-91 (2003). Conserved and non-conserved residues on both the BED and A′GFCC′C″ faces were selected for site-specific mutagenesis. Residues in the mouse molecules were mutated to enable subsequent functional studies of selected mutant proteins. The binding characteristics of the resulting variant polypeptides were assessed by specific ELISA and FACS analysis for binding to PD-1. A total of 17 mB7-DC variants and 21 mB7-H1 variants were prepared and tested. The results are summarized in Tables 1 and 2. Particular residues within mB7-DC and mB7-H1 were only considered to be important for ligand-receptor interactions if their mutation caused at least a 50% loss of binding by FACS, or at least an order of magnitude loss by ELISA.

Mutation of about half of these residues significantly abolished binding to mPD-1. In particular, mB7-DC residues E71, 1105, D111, and K113 were identified as important for binding to mPD1. For mB7-H1, the identified residues were F67, 1115, K124 and 1126. Mutation of residues S58 in mB7-DC and E58, A69 and C113 in mB7-H1 increased binding to mPD-1 as determined by ELISA. Thus, these residues must at least be proximal to the receptor-ligand interface and have not only some tolerance for substitution but also potential optimization of binding interactions.

Variants of human B7-DC were also tested for binding to PD-1 using ELISA and FACS analysis. Mutation of hB7-DC residues K113 and D111 were identified as important for binding to PD-1.

TABLE 1
Summary of amino acid substitutions and binding
characteristics of mouse B7-DC mutants

| | | Substitutions^{b} | | PD-1 binding | |
|---|---|---|---|---|---|
| | | Nucleic | Amino | | ELISA |
| Mutants^{a} | Sites | acids(s) | acid | FACS^{c} | (%)^{d} |
| B7-DC | | | | ++++ | 100 |
| D33S | A′ strand | GAG→AGC | D→S | ++++ | 30 |
| S39Y | B strand | AGC→TAC | S→Y | ++++ | 60 |
| E41S | B strand | GAG→AGC | E→S | ++++ | 100 |
| R56S | C strand | AGA→TCT | R→S | +++/++ | 5 |
| S58Y | C strand | AGT→TAC | S→Y | ++++ | 170 |
| D65S | C′ strand | GAT→AGC | D→S | ++++ | 100 |
| S67Y | C′ strand | TCT→TAC | S→Y | +++/++ | 3 |
| E71S | C″ strand | GAA→AGC | E→S | +++/++ | 2 |
| R72S | C″ strand | AGA→AGC | R→S | ++++ | 60 |
| K84S | D strand | AAG→AGC | K→S | +++/++++ | 13 |
| H88A | E strand | CAC→GCC | H→A | +++/++++ | 20 |
| R101S | F strand | CGT→AGC | R→S | +++ | 7 |
| L103A | F strand | CTG→GCC | L→A | +++ | 25 |
| I105A | F strand | ATC→GCC | I→A | ++ | 0.5 |
| D111S | G strand | GAC→AGC | D→S | ++ | 0.3 |
| K113S | G Strand | AAG→TGC | K→S | −/+ | <0.1 |
| T116Y | G strand | ACG→TAC | T→Y | +++/++++ | 20 |

TABLE 2
Summary of amino acid substitutions and binding characteristics
of mouse B7-H1 mutants

| | | Substitutions^{b} | | Binding activity | |
|---|---|---|---|---|---|
| | | Nucleic | Amino | | ELISA |
| Mutants^{a} | Sites | Acid | Acid | FACS | (%)^{c} |
| B7-H1 | | | | ++++ | 100 |
| L27A | A′ strand | TTG > GCC | Leu > Ala | ++++ | 100 |
| E31S | A′ strand | GAG > AGC | Glu > Ser | ++ | 50 |
| S34Y | B strand | AGC > TAC | Ser > Tyr | ++++ | 60 |
| T37Y | B strand | ACG > TAC | Thr > Tyr | ++ | 5 |
| D49S | B/C strand | GAC > AGC | Asp > Ser | ++++ | 30 |
| Y56S | C strand | TAC > AGC | Tyr > Ser | ++++ | 100 |
| E58S | C strand | GAA > AGC | Glu > Ser | +++++ | 300 |
| E62S | C/C′ strand | GAG > AGC | Glu > Ser | ++++ | 50 |
| F67A | C′ strand | TTT > GCC | Phe > Ala | +/− | 2 |
| A69F | C′ strand | GCA > TTC | Ala > Phe | +++++ | 300 |
| E72S | C′ strand | GAG > AGC | Glu > Ser | ++++ | 60 |
| K75S | C″/D strand | AAG > AGC | Lys > Ser | ++++ | 100 |
| K89S | D strand | AAG > AGC | Lys > Ser | ++++ | 60 |
| A89F | E strand | GCC > TTC | Ala > Phe | ++++ | 40 |
| Q100S | E strand | CAG > AGC | Gln > Ser | ++++ | 100 |
| C113Y | F strand | TGC > TAC | Cys > Tyr | +++++ | 300 |
| I115A | F strand | ATA > GCC | Ile > Ala | +/− | 3 |
| S117Y | F strand | AGC > TAC | Ser > Tyr | ++++ | 100 |
| K124S | G strand | AAG > AGC | Lys > Ser | + | 3 |
| I126A | G strand | ATC > GCC | Ile > Ala | − | 1.4 |
| K129S | G strand | AAA > AGC | Lys > Ser | ++ | 35 |

### Example 2

### B7-DC-Ig Competes with B7-H1 for Binding to PD-1

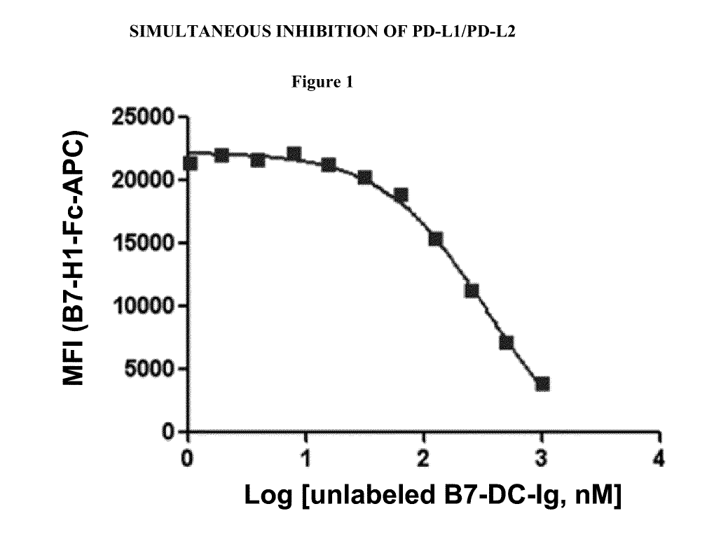

B7-H1-Ig was first conjugated with allophycocyanin (APC). Unlabeled B7-DC-Ig at various concentrations was first incubated with a CHO cell line constitutively expressing PD-1 before adding B7-H1-Ig-APC to the probe and cell mixture. FIG. 1 shows the median fluorescence intensity (MFI) of B7-H1-Ig-APC (y-axis) as a function of the concentration of unlabeled B7-DC-Ig competitor (x-axis) added. As the concentration of unlabeled B7-DC-Ig is increased the amount of B7-H1-Ig-APC bound to CHO cells decreases, demonstrating that B7-DC competes with B7-H1 for binding to PD-1.

### Example 3

### Combination of Cyclophosphamide and B7-Dc-Ig can Generate Tumor Specific, Memory Cytotoxic T Lymphocytes

Balb/C mice at age of 9 to 11 weeks were implanted subcutaneously with 1.0×10⁵ CT26 colorectal tumor cells. On day 10 post tumor implantation, mice received 100 mg/kg of cyclophosphamide. B7-DC-Ig treatment started 1 day later, on day 11. Mice were treated with 100 ug of B7-DC-Ig, 2 doses per week, for 4 weeks and total 8 doses. 75% of the mice that received the CTX+B7-DC-Ig treatment regimen eradicated the established tumors by Day 44, whereas all mice in the control CTX alone group died as a result of tumor growth or were euthanized because tumors exceeded the sizes approved by IACUC.

Mice that eradicated established CT26 colorectal tumors from the above described experiment were rechallenged with 1×10⁵ CT26 cells on Day 44 and Day 70. No tumors grew out from the rechallenge suggesting they had developed long term anti-tumor immunity from the cyclophosphamide and B7-DC-Ig combination treatment. All mice in the vehicle control group developed tumors. This demonstrated the effectiveness of the treatment on established tumors and that the B7-DC-Ig combination treatment resulted in memory responses to tumor antigens.

Mice eradiated established CT26 colorectal tumors from the above described experiment were rechallenged with 2.5×10⁵ CT26 cells on Day 44. Seven days later, mouse spleens were isolated. Mouse splenocytes were pulsed with 5 or 50 ug/mL of ovalbumin (OVA) or AH1 peptides for 6 hours in the presence of a Golgi blocker (BD BioScience). Memory T effector cells were analyzed by assessing CD8+/IFNγ+ T cells.

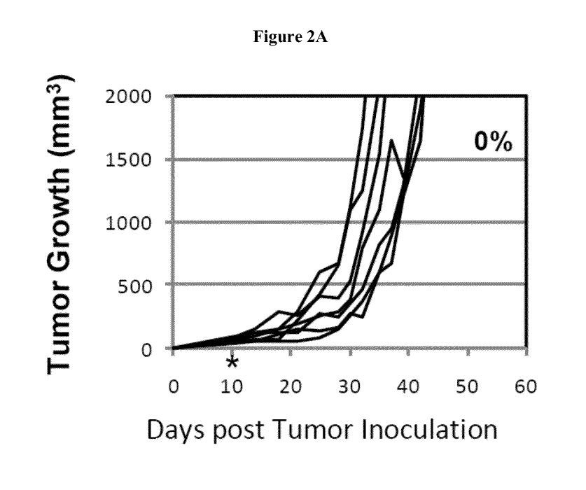

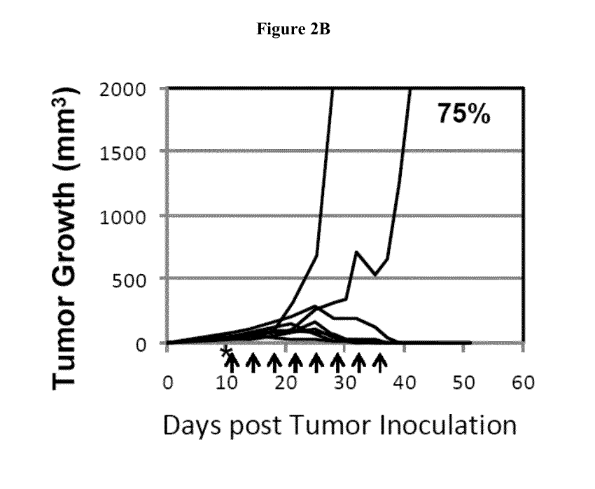

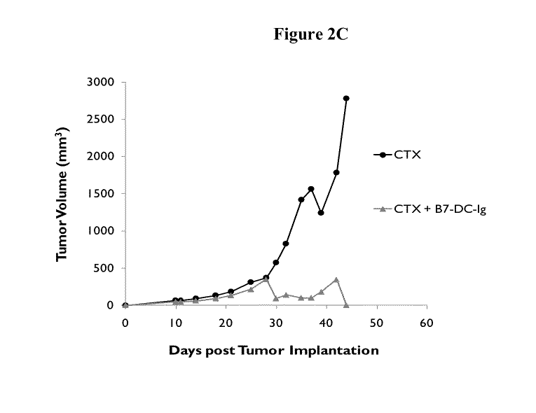

FIGS. 2A-C show the results of experiments wherein the combination of cyclophosphamide (CTX or Cytoxan®) and B7-DC-Ig resulted in eradication of established CT26 tumors (colon carcinoma) in mice. FIG. 2A shows tumor volume (mm³) versus days post tumor challenge in mice treated with 100 mg/kg of CTX on Day 10 while FIG. 2B shows tumor volume (mm³) versus days post tumor challenge in mice treated with CTX on Day 10 followed by B7-DC-Ig administration starting one day later. Each line in each graph represents one mouse. Black arrow stands for B7-DC-Ig administration. FIG. 2C shows average tumor volume for the mice in 2A and 2B.

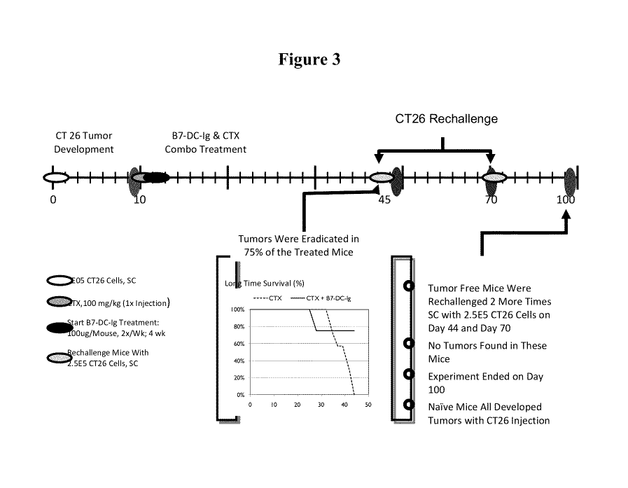

FIG. 3 shows the results of experiments wherein the combination of CTX and B7-DC-Ig eradicated established CT26 tumors (colon carcinoma) in mice and protected against re-challenge with CT26. Mice that were treated with CTX and B7-DC-Ig and found to be free of tumor growth on day 44 following tumor inoculation were rechallenged with tumors. The mice were later rechallenged again on on Day 70. None of the re-challenged mice displayed tumor growth by day 100.

### Example 4

### CTX and B7-DC-Ig Treatment Resulted in Generation of Tumor Specific Memory CTL

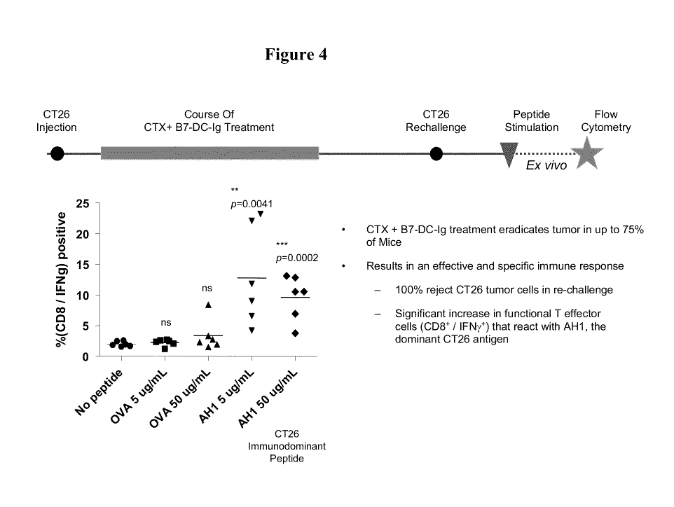

FIG. 4 shows CTX and B7-DC-Ig treatment resulted in generation of tumor specific memory CTL. Mice that eradicated established CT26 subcutenous tumors post CTX and B7-DC-Ig treatment, as described above, were re-challenged with CT26 cells on day 50. Seven days later, splenocytes were isolated and pulsed with either ovalbumin, an irrelevant peptide, or AH1, a CT26 specific peptide. Cells were stained with anti-CD8 antibody first followed by intracellular staining with anti-IFNγ antibody prior to FACS analysis.

FIG. 5 shows the effects of different doses of B7-DC-Ig in combination with CTX on the eradication of established CT26 tumors in mice. Balb/C mice at age of 9 to 11 weeks were implanted subcutaneously with 1.0×10⁵ CT26 cells. On Day 9, mice were injected IP with 100 mg/kg of CTX. Starting on Day 10, mice were treated with 30, 100, or 300 ug of B7-DC-Ig biweekly for 4 weeks. Tumor growth was measured two times per week.

### Example 5

### CTX in B7-DC-Ig Regimen Leads to Significant Reduction of PD-1+CD8+ T Cells in the Tumor Microenvironment

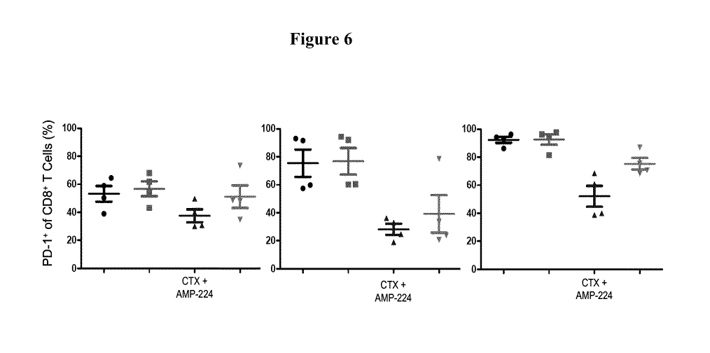

FIGS. 6A-C show the results of experiments where treatment of mice with the CTX and B7-DC-Ig regimen leads to significant reduction of PD-1+CD8+ T cells in the tumor microenvironment. Balb/C mice at age of 9 to 11 weeks of age were implanted with 1×10⁵ CT26 cells subcutaneously. On Day 9, mice were injected with 100 mg/kg of CTX, IP. Starting on Day 10, mice were treated with 100 ug of B7-DC-Ig biweekly for 4 weeks. There were 4 groups: vehicle injected control, CTX alone, CTX+ B7-DC-Ig or B7-DC-Ig alone. Four mice were removed from the study on days 11 (2 days post CTX), 16 (7 days post CTX) and 22 (13 days post CTX) for T cell analysis. FIG. 6A shows that at 2 days post CTX injection, PD-1+/CD8+ T cells were slight lower in the CTX+B7-DC-Ig treated group. FIG. 6B shows that at 7 days post CTX injection, PD-1+/CD8+ T cells were significantly lower in the CTX+B7-DC-Ig treated and B7-DC-Ig alone groups. FIG. 6C shows that at 13 days post CTX injection, PD-1+/CD8+ T cells were significantly lower in the CTX+B7-DC-Ig treated group and slightly lower in the B7-DC-Ig alone group.

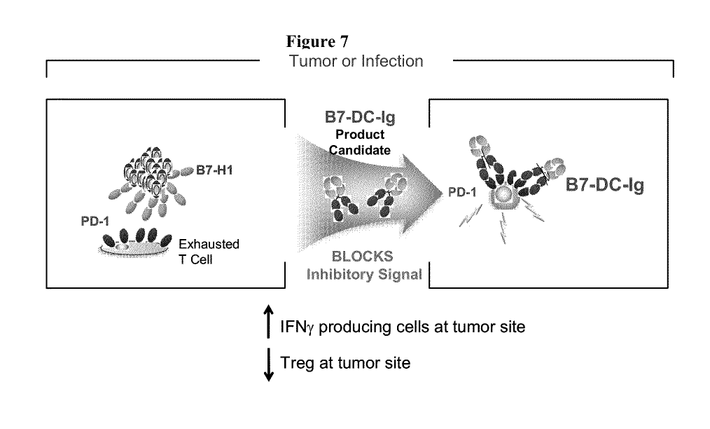

FIG. 7 shows a schematic cartoon of how B7-DC-Ig breaks immune evasion by blocking PD-1 and B7-H1 interaction. B7-DC-Ig can interact with PD-1 expressed on exhausted T cells, preventing B7-H1 binding, and can increase IFNγ producing cells. In addition, binding of B7-DC-Ig to PD-1 prevents binding of PD-L2 and can decrease Treg cells at the tumor site or pathogen infected area.

### Example 6

### Pharmacokinetics in Cynomolgus

Methods and Materials

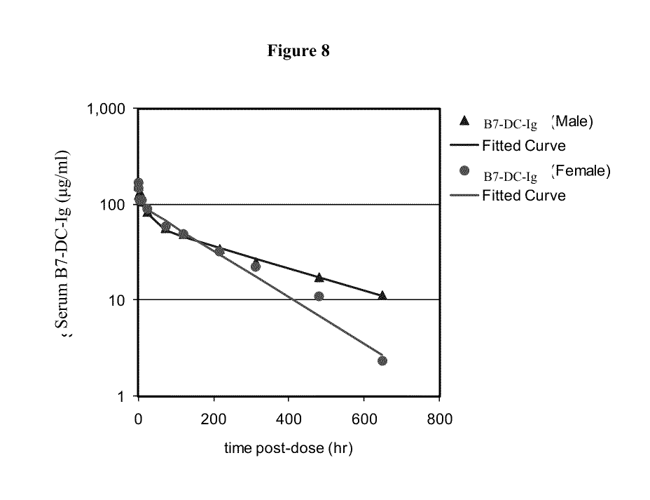

A pilot study incorporating several standard toxicity and immunotoxicity endpoints (i.e., cage side observations, body weight, clinical chemistry, hematology, cytokine release, and immunophenotyping) was performed in cynomolgus monkey with B7-DC-Ig. Two monkeys, one male and one female, were administered 10 mg/kg B7-DC-Ig by IV bolus injection. Cage side observations were recorded 2 hours and 4 hours after injection and twice a day thereafter for 28 days; no abnormalities were noted. Body weights were taken pre-dose and on Study Day 1, 8, and 15; no difference were observed (FIG. 8).

TABLE 3
Pharmacokinetic Parameters for B7-DC-Ig in Cynomolgus Monkey
after Receiving a Single IV Dose at 10 mg/kg

| | Dose level | AUC | Vi | Vss | Cl | T½ |
|---|---|---|---|---|---|---|
| Sex | (mg/kg) | (hr × μg/mL) | (mL/kg) | (mL/kg) | (mL/hr/kg) | (hr) |
| M | 10 | 18,000 | 71 | 140 | 0.40 | 250 |
| F | 10 | 25,000 | 59 | 97 | 0.54 | 120 |

Results

FIG. 8 shows the data fit to two compartmental open pharmacokinetic models with IV bolus input using nonlinear regression analysis. Half-life of B7-DC-Ig was 5-10 days.

### Example 7

### Single-Dose Pharmacokinetics of Murine B7-Dc-Ig

Methods and Materials

A study was carried out to assess the levels of murine B7-DC-Ig in the plasma of healthy mice following a single IP administration. In a preliminary study, BALB/c mice were injected IP with 100, 300, or 900 μg of murine B7-DC-Ig (corresponding to 1.5, 5, and 45 mg/kg) at Day 0 and level of murine B7-DC-Ig in systemic circulation was analyzed at various time points by ELISA.

Results

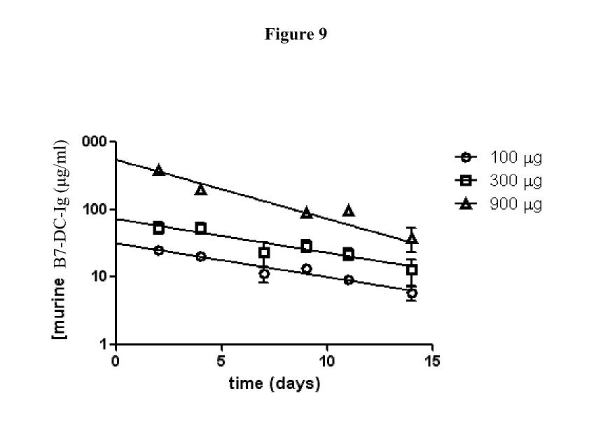

The results of the ELISA assays are shown in FIG. 9. The terminal half-life was estimated to be 3.5 days for the 900 μg dose and 6.0 days for the two lower doses. In conjunction with the dose response and frequency studies described above, plasma levels of murine B7-DC-Ig were measured 6 hours after IP administration of murine B7-DC-Ig (corresponding to Tₘₐₓ) and just before the next administration (corresponding to Tₘᵢₙ). This study was performed twice.

### Example 8

### Repeat Dose Pharmacokinetics of Murine B7-Dc-Ig

Methods and Materials

In conjunction with the dose level and frequency studies summarized in Example 7, the plasma concentration of murine AMP-224 was determined before and after each dose, in two independent studies.

Results

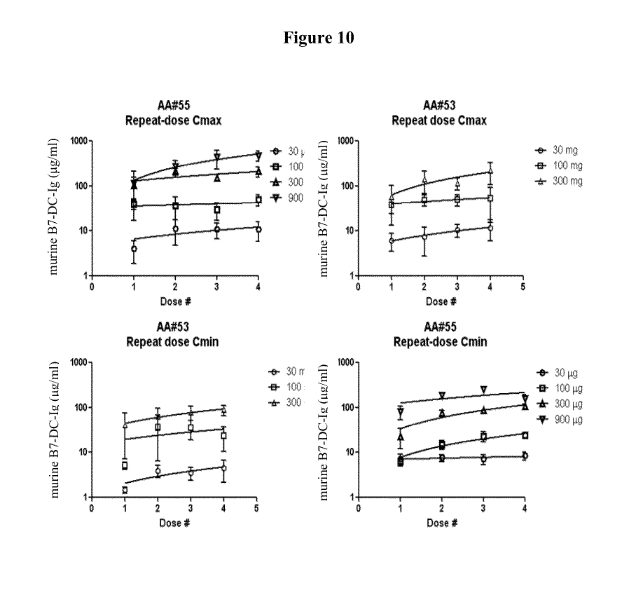

As shown in FIG. 10 and Table 4, the plasma concentration of murine AMP-224 is dependent on the dosage administered. In most groups the concentration of murine AMP-224 is increasing with each dose when it is administered twice a week.

TABLE 4
Plasma concentrations of murine
AMP-224 following repeat dosing.

| | |
|---|---|
| Cₘₐₓ (ng/mL)* | Cₘᵢₙ (ng/mL)* |

| | | | | |
|---|---|---|---|---|
| Dosage | AA#53 | AA#55 | AA#53 | AA#55 |

| | | | | | |
|---|---|---|---|---|---|
| 1.5 | mg/kg | 10 ± 2 | 11 ± 3 | 4 ± 2 | 8 ± 3 |
| 5 | mg/kg | 51 ± 25 | 39 ± 13 | 32 ± 5 | 21 ± 5 |
| 15 | mg/kg | 160 ± 48 | 190 ± 120 | 77 ± 21 | 90 ± 35 |
| 45 | mg/kg | ND | 390 ± 110 | ND | 200 ± 87 |

1. A method of modulating an immune response comprising administering to a subject an effective amount of an immunomodulatory agent to increase IFNγ producing cells and decrease Treg cells at a tumor site or a pathogen infected area of the subject.

2. A method of modulating an immune response comprising administering to a subject an effective amount of an immunomodulatory agent to increase the number of Th17 cells or the level of IL-17 production at a tumor site or a pathogen infected area of the subject.

3. A method of modulating an immune response comprising administering to a subject an effective amount of an immunomodulatory agent to reduce the number of PD-1 positive cells at a tumor site or a pathogen infected area of the subject.

4. The method of claim 1, wherein the immunomodulatory agent simultaneously blocks the binding of endogenous PD-L1 and PD-L2 to PD-1.

5. The method of claim 1, wherein the immunomodulatory agent binds to PD-1.

6. The method of claim 1, wherein the immunomodulatory agent is selected from the group consisting of PD-1, PD-L1, PD-L2, B7.1, fusion proteins thereof and bispecific antibodies that specifically bind to both PD-L1 and PD-L2.

7. The method of claim 1, wherein the immunomodulatory agent binds to PD-1 or a ligand thereof for three months or less after in vivo administration.

8. The method of claim 1, wherein more than one immunomodulatory agent is administered.

9. The method of claim 1, wherein the infection is a chronic viral infection, a bacterial infection, a fungal infection, a mycoplasm infection, a parasitic infection, elicits disease mediated by a toxin during the acute phase of infection or where the infection is characterized by reduced T cell response.

10. The method of claim 9, wherein the viral infection is an infection with a hepatitis virus, a human immunodeficiency virus, a human T-lymphotrophic virus, a herpes virus, an Epstein-Barr virus, filovirus, a human papilloma virus, an Epstein Barr virus, an influenza virus, a respiratory synticial virus, an encephalitis virus, a dengue fever virus, and a papilloma virus.

11. The method of claim 9, wherein the parasitic infection is malaria or Leishmania.

12. The method of claim 9, wherein the bacterial infection is caused by a bacterium selected from the group consisting of Mycobacterium tuberculosis, Bacillus anthracis, Staphylococcus, Listeria, and Clamydia trachomatis.

13. The method of claim 1, further comprising administering a disease antigen in combination with the immunomodulatory agent to enhance an immune response against the disease.

14. The method of claim 1, wherein the immunomodulatory agent is a fusion protein of a PD-1 ligand.

15. The method of claim 14, wherein the PD-1 ligand is a variant PD-1 ligand that has increased affinity for PD-1 as compared to a wild-type PD-1 ligand.

16. The method of claim 14, wherein the fusion protein comprises the extracellular domain of PD-L2 or a fragment thereof capable of binding to PD-1.

17. The method of claim 16, wherein the fusion protein has an amino acid sequence according to SEQ ID NO:60.

18. The method of claim 1, further comprising administering with the immunomodulatory agent an additional active agent selected from the group consisting of immunomodulators, agents that deplete or inhibit the function of Tregs, and costimulatory molecules.

19. The method of claim 18, wherein the additional active agent is an agent that depletes or inhibits the function of CD4+CD25+ Tregs.

20. The method of claim 18, wherein the agent that depletes or inhibits the function of CD4+CD25+ Tregs is cyclophosphamide.

21. The method of claim 1 any of for enhancing antigen presenting cell function comprising contacting APCs with a immunomodulatory agent in an amount effective to inhibit, reduce, or block PD-1 signal transduction in the APCs or enhance clearance of diseased or infected cells.

22. The method of claim 1, wherein the tumor is selected from the group consisting of sarcoma, melanoma, lymphoma, neuroblastoma, and carcinoma.

23. A composition comprising an immunomodulatory agent that increases IFNγ producing cells and decreases Treg cells at a tumor site or a pathogen infected area of a subject in combination with one or more disease antigens.

24. A composition comprising an immunomodulatory agent that increases IFNγ producing cells and decreases Treg cells at a tumor site or a pathogen infected area of a subject in combination with a vaccine.


SEQUENCE LISTING (71 sequences)

SEQ ID NO: 1 - protein, 247 aa, Mus musculus
MLLLLPILNLSLQLHPVAALFTVTAPKEVYTVDVGSSVSLECDFDRRECTELEGIRASLQKVENDTSLQSERATLLEEQLPLGKALFHIPSVQVRDSGQYRCLVICGAAWDYKYLTVKVKASYMRIDTRILEVPGTGEVQLTCQARGYPLAEVSWQNVSVPANTSHIRTPEGLYQVTSVLRLKPQPSRNFSCMFWNAHMKELTSAIIDPLSRMEPKVPRTWPLHVFIPACTIALIFLAIVIIQRKRI

SEQ ID NO: 2 - protein, 228 aa, Mus musculus
LFTVTAPKEVYTVDVGSSVSLECDFDRRECTELEGIRASLQKVENDTSLQSERATLLEEQLPLGKALFHIPSVQVRDSGQYRCLVICGAAWDYKYLTVKVKASYMRIDTRILEVPGTGEVQLTCQARGYPLAEVSWQNVSVPANTSHIRTPEGLYQVTSVLRLKPQPSRNFSCMFWNAHMKELTSAIIDPLSRMEPKVPRTWPLHVFIPACTIALIFLAIVIIQRKRI

SEQ ID NO: 3 - protein, 273 aa, Homo sapien
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 4 - protein, 254 aa, Homo sapien
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 5 - protein, 273 aa, Macaca fascicularis
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPSCIIAFIFIATVIALRKQLCQKLYSSKDATKRPVTTTKREVNSAI

SEQ ID NO: 6 - protein, 254 aa, Macaca fascicularis
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPSCIIAFIFIATVIALRKQLCQKLYSSKDATKRPVTTTKREVNSAI

SEQ ID NO: 7 - protein, 290 aa, Mus musculus
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHWVLLGSILLFLIVVSTVLLFLRKQVRMLDVEKCGVEDTSSKNRNDTQFEET

SEQ ID NO: 8 - protein, 272 aa, Mus musculus [sequence omitted]

SEQ ID NO: 9 - protein, 290 aa, Homo sapien
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 10 - protein, 272 aa, Homo sapien
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 11 - protein, 306 aa, Mus musculus
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 12 - protein, 269 aa, Mus musculus
VDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 13 - protein, 288 aa, Homo sapien
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 14 - protein, 254 aa, Homo sapien
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 15 - protein, 288 aa, Homo sapien
MQIPQAPWPVVWAVLQLGWRPGWFLDSPDRPWNPPTFFPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 16 - protein, 288 aa, Macaca fascicularis
MQIPQAPWPVVWAVLQLGWRPGWFLESPDRPWNAPTFSPALLLVTEGDNATFTCSFSNASESFVLNWYRMSPSNQTDKLAAFPEDRSQPGQDCRFRVTRLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 17 - protein, 288 aa, Mus musculus
MWVRQVPWSFTWAVLQLSWQSGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMVIGIMSALVGIPVLLLLAWALAVFCSTSMSEARGAGSKDDTLKEEPSAAPVPSVAYEELDFQGREKTPELPTACVHTEYATIVFTEGLGASAMGRRGSADGLQGPRPPRHEDGHCSWPL

SEQ ID NO: 18 - DNA, 663 bp, Homo sapien
atgatctttcttctcttgatgctgtctttggaattgcaacttcaccaaatcgcggccctctttactgtgaccgtgccaaaagaactgtatatcattgagcacgggtccaatgtgaccctcgaatgtaactttgacaccggcagccacgttaacctgggggccatcactgccagcttgcaaaaagttgaaaacgacacttcacctcaccgggagagggcaaccctcttggaggagcaactgccattggggaaggcctcctttcatatccctcaggtgcaggttcgggatgagggacagtaccagtgcattattatctacggcgtggcttgggattacaagtatctgaccctgaaggtgaaagcgtcctatcggaaaattaacactcacattcttaaggtgccagagacggacgaggtggaactgacatgccaagccaccggctacccgttggcagaggtcagctggcccaacgtgagcgtacctgctaacacttctcattctaggacacccgagggcctctaccaggttacatccgtgctccgcctcaaaccgcccccaggccggaattttagttgcgtgttttggaatacccacgtgcgagagctgactcttgcatctattgatctgcagtcccagatggagccacggactcatccaacttgg

SEQ ID NO: 19 - protein, 261 aa, Homo sapien
MIFLLLMLSLELQLHQIAALFTVTVPKELYIIEHGSNVTLMIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTW

SEQ ID NO: 20 - protein, 202 aa, Homo sapien
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTW

SEQ ID NO: 21 - DNA, 294 bp, Homo sapiens
tttactgtgaccgtgccaaaagaactgtatatcattgagcacgggtccaatgtgaccctcgaatgtaactttgacaccggcagccacgttaacctgggggccatcactgccagcttgcaaaaagttgaaaacgacacttcacctcaccgggagagggcaaccctcttggaggagcaactgccattggggaaggcctcctttcatatccctcaggtgcaggttcgggatgagggacagtaccagtgcattattatctacggcgtggcttgggattacaagtatctgaccctgaag

SEQ ID NO: 22 - protein, 98 aa, Homo sapien
FTVTVPKELYIIEHGSNVTLECNFDTGSHVNLGAITASLQKVENDTSPHRERATLLEEQLPLGKASFHIPQVQVRDEGQYQCIIIYGVAWDYKYLTLK

SEQ ID NO: 23 - DNA, 663 bp, Macaca fascicularis
atgatcttcctcctgctaatgttgagcctggaattgcagcttcaccagatagcagctttattcacagtgacagtccctaaggaactgtacataatagagcatggcagcaatgtgaccctggaatgcaactttgacactggaagtcatgtgaaccttggagcaataacagccagtttgcaaaaggtggaaaatgatacatccccacaccgtgaaagagccactttgctggaggagcagctgcccctagggaaggcctcgttccacatacctcaagtccaagtgagggacgaaggacagtaccaatgcataatcatctatggggtcgcctgggactacaagtacctgactctgaaagtcaaagcttcctacaggaaaataaacactcacatcctaaaggttccagaaacagatgaggtagagctcacctgccaggctacaggttatcctctggcagaagtatcctggccaaacgtcagcgttcctgccaacaccagccactccaggacccctgaaggcctctaccaggtcaccagtgttctgcgcctaaagccaccccctggcagaaacttcagctgtgtgttctggaatactcacgtgagggaacttactttggccagcattgaccttcaaagtcagatggaacccaggacccatccaacttgg

SEQ ID NO: 24 - protein, 221 aa, Macaca fascicularis
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTW

SEQ ID NO: 25 - protein, 202 aa, Macaca fascicularis; identical to SEQ ID NO: 20

SEQ ID NO: 26 - DNA, 294 bp, Macaca fascicularis
ttcacagtgacagtccctaaggaactgtacataatagagcatggcagcaatgtgaccctggaatgcaactttgacactggaagtcatgtgaaccttggagcaataacagccagtttgcaaaaggtggaaaatgatacatccccacaccgtgaaagagccactttgctggaggagcagctgcccctagggaaggcctcgttccacatacctcaagtccaagtgagggacgaaggacagtaccaatgcataatcatctatggggtcgcctgggactacaagtacctgactctgaaa

SEQ ID NO: 27 - protein, 98 aa, Macaca fascicularis; identical to SEQ ID NO: 22

SEQ ID NO: 28 - DNA, 663 bp, Mus musculus
atgctgctcctgctgccgatactgaacctgagcttacaacttcatcctgtagcagctttattcaccgtgacagcccctaaagaagtgtacaccgtagacgtcggcagcagtgtgagcctggagtgcgattttgaccgcagagaatgcactgaactggaagggataagagccagtttgcagaaggtagaaaatgatacgtctctgcaaagtgaaagagccaccctgctggaggagcagctgcccctgggaaaggctttgttccacatccctagtgtccaagtgagagattccgggcagtaccgttgcctggtcatctgcggggccgcctgggactacaagtacctgacggtgaaagtcaaagcttcttacatgaggatagacactaggatcctggaggttccaggtacaggggaggtgcagcttacctgccaggctagaggttatcccctagcagaagtgtcctggcaaaatgtcagtgttcctgccaacaccagccacatcaggacccccgaaggcctctaccaggtcaccagtgttctgcgcctcaagcctcagcctagcagaaacttcagctgcatgttctggaatgctcacatgaaggagctgacttcagccatcattgaccctctgagtcggatggaacccaaagtccccagaacgtgg

SEQ ID NO: 29 - protein, 221 aa, Mus musculus
MLLLLPILNLSLQLHPVAALFTVTAPKEVYTVDVGSSVSLECDFDRRECTELEGIRASLQKVENDTSLQSERATLLEEQLPLGKALFHIPSVQVRDSGQYRCLVICGAAWDYKYLTVKVKASYMRIDTRILEVPGTGEVQLTCQARGYPLAEVSWQNVSVPANTSHIRTPEGLYQVTSVLRLKPQPSRNFSCMFWNAHMKELTSAIIDPLSRMEPKVPRTW

SEQ ID NO: 30 - protein, 202 aa, Mus musculus
LFTVTAPKEVYTVDVGSSVSLECDFDRRECTELEGIRASLQKVENDTSLQSERATLLEEQLPLGKALFHIPSVQVRDSGQYRCLVICGAAWDYKYLTVKVKASYMRIDTRILEVPGTGEVQLTCQARGYPLAEVSWQNVSVPANTSHIRTPEGLYQVTSVLRLKPQPSRNFSCMFWNAHMKELTSAIIDPLSRMEPKVPRTW

SEQ ID NO: 31 - DNA, 294 bp, Mus musculus
ttcaccgtgacagcccctaaagaagtgtacaccgtagacgtcggcagcagtgtgagcctggagtgcgattttgaccgcagagaatgcactgaactggaagggataagagccagtttgcagaaggtagaaaatgatacgtctctgcaaagtgaaagagccaccctgctggaggagcagctgcccctgggaaaggctttgttccacatccctagtgtccaagtgagagattccgggcagtaccgttgcctggtcatctgcggggccgcctgggactacaagtacctgacggtgaaa

SEQ ID NO: 32 - protein, 98 aa, Mus musculus
FTVTAPKEVYTVDVGSSVSLECDFDRRECTELEGIRASLQKVENDTSLQSERATLLEEQLPLGKALFHIPSVQVRDSGQYRCLVICGAAWDYKYLTVK

SEQ ID NO: 33 - protein, 220 aa, Homo sapien
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 34 - protein, 239 aa, Homo sapien
FTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHWVLLGSILLFLIVVSTVL

SEQ ID NO: 35 - DNA, 738 bp, Mus musculus
atggcttgcaattgtcagttgatgcaggatacaccactcctcaagtttccatgtccaaggctcattcttctctttgtgctgctgattcgtctttcacaagtgtcttcagatgttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttggctttagtaaagttgtccatcaaagctgacttctctacccccaacataactgagtctggaaacccatctgcagacactaaaaggattacctgctttgcttccgggggtttcccaaagcctcgcttctcttggttggaaaatggaagagaattacctggcatcaatacgacaatttcccaggatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaac

SEQ ID NO: 36 - protein, 246 aa, Mus musculus
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKN

SEQ ID NO: 37 - protein, 209 aa, Mus musculus
VDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKN

SEQ ID NO: 38 - DNA, 291 bp, Mus musculus
gttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttg

SEQ ID NO: 39 - protein, 97 aa, Mus musculus
VDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHL

SEQ ID NO: 40 - DNA, 732 bp, Homo sapien
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctc

SEQ ID NO: 41 - protein, 243 aa, Homo sapien
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNL

SEQ ID NO: 42 - protein, 209 aa, Homo sapien
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNL

SEQ ID NO: 43 - DNA, 303 bp, Homo sapien
gttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacg

SEQ ID NO: 44 - protein, 101 aa, Homo sapien
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVT

SEQ ID NO: 45 - protein, 150 aa, Homo sapien [sequence omitted]

SEQ ID NO: 46 - DNA, 696 bp, Homo sapiens
gagcctaagtcatgtgacaagacccatacgtgcccaccctgtcccgctccagaactgctggggggacctagcgttttcttgttccccccaaagcccaaggacaccctcatgatctcacggactcccgaagtaacatgcgtagtagtcgacgtgagccacgaggatcctgaagtgaagtttaattggtacgtggacggagtcgaggtgcataatgccaaaactaaacctcgggaggagcagtataacagtacctaccgcgtggtatccgtcttgacagtgctccaccaggactggctgaatggtaaggagtataaatgcaaggtcagcaacaaagctcttcccgccccaattgaaaagactatcagcaaggccaagggacaaccccgcgagccccaggtttacacccttccaccttcacgagacgagctgaccaagaaccaggtgtctctgacttgtctggtcaaaggtttctatccttccgacatcgcagtggagtgggagtcaaacgggcagcctgagaataactacaagaccacacccccagtgcttgatagcgatgggagctttttcctctacagtaagctgactgtggacaaatcccgctggcagcagggaaacgttttctcttgtagcgtcatgcatgaggccctccacaaccattatactcagaaaagcctgagtctgagtcccggcaaa

SEQ ID NO: 47 - protein, 231 aa, Homo sapien
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 330 aa, Homo sapien [sequence omitted]

SEQ ID NO: 49 - DNA, 699 bp, Mus musculus
gagccaagaggtcctacgatcaagccctgcccgccttgtaaatgcccagctccaaatttgctgggtggaccgtcagtctttatcttcccgccaaagataaaggacgtcttgatgattagtctgagccccatcgtgacatgcgttgtggtggatgtttcagaggatgaccccgacgtgcaaatcagttggttcgttaacaacgtggaggtgcataccgctcaaacccagacccacagagaggattataacagcaccctgcgggtagtgtccgccctgccgatccagcatcaggattggatgagcgggaaagagttcaagtgtaaggtaaacaacaaagatctgccagcgccgattgaacgaaccattagcaagccgaaagggagcgtgcgcgcacctcaggtttacgtccttcctccaccagaagaggagatgacgaaaaagcaggtgaccctgacatgcatggtaactgactttatgccagaagatatttacgtggaatggactaataacggaaagacagagctcaattacaagaacactgagcctgttctggattctgatggcagctactttatgtactccaaattgagggtcgagaagaagaattgggtcgagagaaacagttatagttgctcagtggtgcatgagggcctccataatcatcacaccacaaagtccttcagccgaacgcccgggaaa

SEQ ID NO: 50 - protein, 233 aa, Mus musculus
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 51 - protein, 4 aa, Artificial Sequence
  Synthetic Peptide Linker
GSGS

SEQ ID NO: 52 - protein, 4 aa, Artificial Sequence
  Synthetic Peptide Linker
GGGS

SEQ ID NO: 53 - protein, 15 aa, Artificial Sequence
  Synthetic Peptide Linker
GGGGSGGGGSGGGGS

SEQ ID NO: 54 - protein, 20 aa, Artificial Sequence
  Synthetic Peptide Linker
GGGGSGGGGSGGGGSGGGGS

SEQ ID NO: 55 - DNA, 1365 bp, Artificial Sequence
  Murine PD-L2 Fusion Protein
atgctgctcctgctgccgatactgaacctgagcttacaacttcatcctgtagcagctttattcaccgtgacagcccctaaagaagtgtacaccgtagacgtcggcagcagtgtgagcctggagtgcgattttgaccgcagagaatgcactgaactggaagggataagagccagtttgcagaaggtagaaaatgatacgtctctgcaaagtgaaagagccaccctgctggaggagcagctgcccctgggaaaggctttgttccacatccctagtgtccaagtgagagattccgggcagtaccgttgcctggtcatctgcggggccgcctgggactacaagtacctgacggtgaaagtcaaagcttcttacatgaggatagacactaggatcctggaggttccaggtacaggggaggtgcagcttacctgccaggctagaggttatcccctagcagaagtgtcctggcaaaatgtcagtgttcctgccaacaccagccacatcaggacccccgaaggcctctaccaggtcaccagtgttctgcgcctcaagcctcagcctagcagaaacttcagctgcatgttctggaatgctcacatgaaggagctgacttcagccatcattgaccctctgagtcggatggaacccaaagtccccagaacgtgggagccaagaggtcctacgatcaagccctgcccgccttgtaaatgcccagctccaaatttgctgggtggaccgtcagtctttatcttcccgccaaagataaaggacgtcttgatgattagtctgagccccatcgtgacatgcgttgtggtggatgtttcagaggatgaccccgacgtgcaaatcagttggttcgttaacaacgtggaggtgcataccgctcaaacccagacccacagagaggattataacagcaccctgcgggtagtgtccgccctgccgatccagcatcaggattggatgagcgggaaagagttcaagtgtaaggtaaacaacaaagatctgccagcgccgattgaacgaaccattagcaagccgaaagggagcgtgcgcgcacctcaggtttacgtccttcctccaccagaagaggagatgacgaaaaagcaggtgaccctgacatgcatggtaactgactttatgccagaagatatttacgtggaatggactaataacggaaagacagagctcaattacaagaacactgagcctgttctggattctgatggcagctactttatgtactccaaattgagggtcgagaagaagaattgggtcgagagaaacagttatagttgctcagtggtgcatgagggcctccataatcatcacaccacaaagtccttcagccgaacgcccgggaaatga

SEQ ID NO: 56 - protein, 454 aa, Artificial Sequence
  Murine PD-L2 Fusion Protein
MLLLLPILNLSLQLHPVAALFTVTAPKEVYTVDVGSSVSLECDFDRRECTELEGIRASLQKVENDTSLQSERATLLEEQLPLGKALFHIPSVQVRDSGQYRCLVICGAAWDYKYLTVKVKASYMRIDTRILEVPGTGEVQLTCQARGYPLAEVSWQNVSVPANTSHIRTPEGLYQVTSVLRLKPQPSRNFSCMFWNAHMKELTSAIIDPLSRMEPKVPRTWEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 57 - protein, 435 aa, Artificial Sequence
  Murine PD-L2 Fusion Protein
LFTVTAPKEVYTVDVGSSVSLECDFDRRECTELEGIRASLQKVENDTSLQSERATLLEEQLPLGKALFHIPSVQVRDSGQYRCLVICGAAWDYKYLTVKVKASYMRIDTRILEVPGTGEVQLTCQARGYPLAEVSWQNVSVPANTSHIRTPEGLYQVTSVLRLKPQPSRNFSCMFWNAHMKELTSAIIDPLSRMEPKVPRTWEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 58 - DNA, 1362 bp, Artificial Sequence
  Human PD-L2 Fusion Protein
atgatctttcttctcttgatgctgtctttggaattgcaacttcaccaaatcgcggccctctttactgtgaccgtgccaaaagaactgtatatcattgagcacgggtccaatgtgaccctcgaatgtaactttgacaccggcagccacgttaacctgggggccatcactgccagcttgcaaaaagttgaaaacgacacttcacctcaccgggagagggcaaccctcttggaggagcaactgccattggggaaggcctcctttcatatccctcaggtgcaggttcgggatgagggacagtaccagtgcattattatctacggcgtggcttgggattacaagtatctgaccctgaaggtgaaagcgtcctatcggaaaattaacactcacattcttaaggtgccagagacggacgaggtggaactgacatgccaagccaccggctacccgttggcagaggtcagctggcccaacgtgagcgtacctgctaacacttctcattctaggacacccgagggcctctaccaggttacatccgtgctccgcctcaaaccgcccccaggccggaattttagttgcgtgttttggaatacccacgtgcgagagctgactcttgcatctattgatctgcagtcccagatggagccacggactcatccaacttgggaacctaaatcttgcgataaaactcatacctgtcccccttgcccagcccccgagcttctgggaggtcccagtgtgtttctgtttcccccaaaacctaaggacacacttatgatatcccgaacgccggaagtgacatgcgtggttgtggacgtctcacacgaagacccggaggtgaaattcaactggtacgttgacggagttgaggttcataacgctaagaccaagcccagagaggagcaatacaattccacctatcgagtggttagtgtactgaccgttttgcaccaagactggctgaatggaaaagaatacaagtgcaaagtatcaaacaaggctttgcctgcacccatcgagaagacaatttctaaagccaaagggcagcccagggaaccgcaggtgtacacactcccaccatcccgcgacgagctgacaaagaatcaagtatccctgacctgcctggtgaaaggcttttacccatctgacattgccgtggaatgggaatcaaatggacaacctgagaacaactacaaaaccactccacctgtgcttgacagcgacgggtcctttttcctgtacagtaagctcactgtcgataagtctcgctggcagcagggcaacgtcttttcatgtagtgtgatgcacgaagctctgcacaaccattacacccagaagtctctgtcactgagcccaggtaaatga

SEQ ID NO: 59 - protein, 453 aa, Artificial Sequence
  Human PD-L2 Fusion Protein
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 434 aa, Artificial Sequence
  Human PD-L2 Fusion Protein
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 453 aa, Artificial Sequence; identical to SEQ ID NO: 59
  Non-human Primate PD-L2 Fusion Protein

SEQ ID NO: 62 - protein, 434 aa, Artificial Sequence; identical to SEQ ID NO: 60
  Non-human Primate PD-L2 Fusion Protein

SEQ ID NO: 63 - DNA, 1398 bp, Artificial Sequence
  Human PD-L1 Fusion Protein
atgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaagggacaagacccatacgtgcccaccctgtcccgctccagaactgctggggggacctagcgttttcttgttccccccaaagcccaaggacaccctcatgatctcacggactcccgaagtaacatgcgtagtagtcgacgtgagccacgaggatcctgaagtgaagtttaattggtacgtggacggagtcgaggtgcataatgccaaaactaaacctcgggaggagcagtataacagtacctaccgcgtggtatccgtcttgacagtgctccaccaggactggctgaatggtaaggagtataaatgcaaggtcagcaacaaagctcttcccgccccaattgaaaagactatcagcaaggccaagggacaaccccgcgagccccaggtttacacccttccaccttcacgagacgagctgaccaagaaccaggtgtctctgacttgtctggtcaaaggtttctatccttccgacatcgcagtggagtgggagtcaaacgggcagcctgagaataactacaagaccacacccccagtgcttgatagcgatgggagctttttcctctacagtaagctgactgtggacaaatcccgctggcagcagggaaacgttttctcttgtagcgtcatgcatgaggccctccacaaccattatactcagaaaagcctgagtctgagtcccggcaaatga

SEQ ID NO: 64 - protein, 465 aa, Artificial Sequence
  Human PD-L1 Fusion Protein
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 445 aa, Artificial Sequence
  Human PD-L1 Fusion Protein
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - DNA, 1419 bp, Artificial Sequence
  Murine PD-L1 Fusion Protein
atgaggatatttgctggcattatattcacagcctgctgtcacttgctacgggcgtttactatcacggctccaaaggacttgtacgtggtggagtatggcagcaacgtcacgatggagtgcagattccctgtagaacgggagctggacctgcttgcgttagtggtgtactgggaaaaggaagatgagcaagtgattcagtttgtggcaggagaggaggaccttaagcctcagcacagcaacttcagggggagagcctcgctgccaaaggaccagcttttgaagggaaatgctgcccttcagatcacagacgtcaagctgcaggacgcaggcgtttactgctgcataatcagctacggtggtgcggactacaagcgaatcacgctgaaagtcaatgccccataccgcaaaatcaaccagagaatttccgtggatccagccacttctgagcatgaactaatatgtcaggccgagggttatccagaagctgaggtaatctggacaaacagtgaccaccaacccgtgagtgggaagagaagtgtcaccacttcccggacagaggggatgcttctcaatgtgaccagcagtctgagggtcaacgccacagcgaatgatgttttctactgtacgttttggagatcacagccagggcaaaaccacacagcggagctgatcatcccagaactgcctgcaacacatcctccacagaacaggactcacgagccaagaggtcctacgatcaagccctgcccgccttgtaaatgcccagctccaaatttgctgggtggaccgtcagtctttatcttcccgccaaagataaaggacgtcttgatgattagtctgagccccatcgtgacatgcgttgtggtggatgtttcagaggatgaccccgacgtgcaaatcagttggttcgttaacaacgtggaggtgcataccgctcaaacccagacccacagagaggattataacagcaccctgcgggtagtgtccgccctgccgatccagcatcaggattggatgagcgggaaagagttcaagtgtaaggtaaacaacaaagatctgccagcgccgattgaacgaaccattagcaagccgaaagggagcgtgcgcgcacctcaggtttacgtccttcctccaccagaagaggagatgacgaaaaagcaggtgaccctgacatgcatggtaactgactttatgccagaagatatttacgtggaatggactaataacggaaagacagagctcaattacaagaacactgagcctgttctggattctgatggcagctactttatgtactccaaattgagggtcgagaagaagaattgggtcgagagaaacagttatagttgctcagtggtgcatgagggcctccataatcatcacaccacaaagtccttcagccgaacgcccgggaaatga

SEQ ID NO: 67 - protein, 472 aa, Artificial Sequence
  Murine PD-L1 Fusion Protein
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 68 - protein, 375 aa, Artificial Sequence
  PD-1 Fusion Protein
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - DNA, 1215 bp, Artificial Sequence
  Non-human Primate PD-1 Fusion Protein
atgcagatcccgcaagccccatggcccgttgtatgggcggttcttcaacttggatggagaccaggctggtttctggagagccccgaccggccctggaatgcgccaacgttcagccctgccctcctcttggtgaccgagggtgataacgctaccttcacctgctcatttagtaacgcctctgagtcttttgtcctcaattggtaccggatgagtcccagcaaccagactgataaactggctgcatttccggaggacaggtcccagcctgggcaagactgtaggttccgcgtgaccagactgcctaacggacgcgacttccacatgagtgtcgtgcgagccaggcgcaatgactccggaacttatctctgcggtgccatttccctggcacctaaagctcagataaaggaatctttgagagcagagctgcgcgtgacagaaaggcgggcagaagtgcccacagctcatccgtcacctagccccagaccagcggggcagtttcaaatcgaaggcagaatggatcctaagtcatgtgacaagacccatacgtgcccaccctgtcccgctccagaactgctggggggacctagcgttttcttgttccccccaaagcccaaggacaccctcatgatctcacggactcccgaagtaacatgcgtagtagtcgacgtgagccacgaggatcctgaagtgaagtttaattggtacgtggacggagtcgaggtgcataatgccaaaactaaacctcgggaggagcagtataacagtacctaccgcgtggtatccgtcttgacagtgctccaccaggactggctgaatggtaaggagtataaatgcaaggtcagcaacaaagctcttcccgccccaattgaaaagactatcagcaaggccaagggacaaccccgcgagccccaggtttacacccttccaccttcacgagacgagctgaccaagaaccaggtgtctctgacttgtctggtcaaaggtttctatccttccgacatcgcagtggagtgggagtcaaacgggcagcctgagaataactacaagaccacacccccagtgcttgatagcgatgggagctttttcctctacagtaagctgactgtggacaaatcccgctggcagcagggaaacgttttctcttgtagcgtcatgcatgaggccctccacaaccattatactcagaaaagcctgagtctgagtcccggcaaatga

SEQ ID NO: 70 - protein, 404 aa, Artificial Sequence
  Non-human Primate PD-1 Fusion Protein
MQIPQAPWPVVWAVLQLGWRPGWFLESPDRPWNAPTFSPALLLVTEGDNATFTCSFSNASESFVLNWYRMSPSNQTDKLAAFPEDRSQPGQDCRFRVTRLPNGRDFHMSVVRARRNDSGTYLCGAISLAPKAQIKESLRAELRVTERRAEVPTAHPSPSPRPAGQFQIEGRMDPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 467 aa, Artificial Sequence
  B7.1 Fusion Protein
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK